(19) United States
(12) Patent Application Publication
(10) Pub. No.: US 2009/0258069 A1
(43) Pub. Date: Oct. 15, 2009

(54) Delivery of LFA-1 antagonists to the gastrointestinal system

(76) Inventors: John Burnier, Pacifica, CA (US); Thomas Gadek, Oakland, CA (US)

Correspondence Address: WILSON, SONSINI, GOODRICH & ROSATI, 650 PAGE MILL ROAD, PALO ALTO, CA, 94304-1050, US

(21) Appl. No.: 12/386,347

(22) Filed: Apr. 15, 2009

Related U.S. Application Data
(60) Provisional application No. 61/045,165, filed on Apr. 15, 2008.

(51) Int. Cl. A61K 9/14; A61K 39/395; A61K 33/30; A61K 33/06; A61K 33/00; A61K 31/56; A61K 31/437; A61K 31/47; A61K 31/165; A61K 31/4725

(52) U.S. Cl. 424/489; 424/133.1; 424/641; 424/682; 424/722; 514/171; 514/300; 514/307; 514/617

(57) ABSTRACT

The present invention provides compositions and methods for treating disorders and diseases by delivery of LFA-1 antagonists to the gastrointestinal system. Methods include delivery of LFA-1 antagonists to effect localized treatment.

### REFERENCE

This application claims the benefit of U.S. Provisional Application No. 61/045,165, filed Apr. 15, 2008, which application is incorporated herein by reference.

### CROSS-REFERENCE

Cross reference is made to co-pending applications U.S. application Ser. No. 12/288,330, filed on Oct. 17, 2008; Attorney Docket No. WSGR 32411-708.201, filed on Apr. 15, 2009; Attorney Docket No. WSGR-32411-709.201, filed on Apr. 15, 2009; and Attorney Docket No. WSGR 32411-712.201, filed on Apr. 15, 2009, which are hereby incorporated by reference in their entirety.

### BACKGROUND OF THE INVENTION

The (CD11/CD18) family of adhesion receptor molecules comprises four highly related cell surface glycoproteins; LFA-1 (CD11a/CD18), Mac-1 (CD11b/CD18), p150.95 (CD11c/CD18) and (CD11d/CD18). The CD11/CD18 family is related structurally and genetically to the larger integrin family of receptors that modulate cell adhesive interactions, which include; embryogenesis, adhesion to extracellular substrates, and cell differentiation (Hynes, R. O., Cell 48:549-554 (1987); Kishimoto et al., Adv. Immunol. 46:149-182 (1989); Kishimoto et al., Cell 48:681-690 (1987); Ruoslahti et al., Science 238:491-497 (1987)). LFA-1 is a heterodimeric adhesion molecule present on the surface of all mature leukocytes except a subset of macrophages and is considered the major lymphoid integrin. The expression of Mac-1, p150.95 and CD11d/CD18 is predominantly confined to cells of the myeloid lineage (which include neutrophils, monocytes, macrophage and mast cells). LFA-1 and Mac-1 (CD11b/CD18) are known to be of primary importance to function of leukocytes (Li et al. (2006) Am J Pathology 169:1590-1600). LFA-1 in particular is involved in migration of leukocytes to sites of inflammation (Green et al. (2006) Blood 107:2101-11).

Functional studies have suggested that LFA-1 interacts with several ligands, including ICAM-1 (Rothlein et al., J. Immunol. 137:1270-1274 (1986), ICAM-2, (Staunton et al., Nature 339:361-364 (1989)), ICAM-3 (Fawcett et al., Nature 360:481-484 (1992); Vezeux et al., Nature 360:485-488, (1-992); de Fougerolles and Springer, J. Exp. Med. 175:185-190 (1990)) and Telencephalin (Tian et al., J. Immunol. 158:928-936 (1997)). Normal interaction of LFA-1 with ICAMs act as costimulatory molecules in the peptide-MHC complex (Grakoui et al. (1999) Science 285:221-7; Malissen (1999) Science 285:207-8). ICAMs 1-3 are known to regulate lymphocytes and T-cell activation (Perez et al. (2007) BMC Immunol. 8:2). ICAM-4 is a red blood cell specific ligand and ICAM-5 is known to recruit leukocytes to neurons in the central nervous system (Ihanus et al. (2007) Blood 109:802-10; Tian et al. (2000) Eur J. Immunol. 30:810-8). Upon binding, LFA-1 undergoes a conformational change that results in higher affinity binding and receptor clustering (Hogg et al. (2003) J Cell Sci. 116:4695-705; Takagi et al. (2002) Cell 110:599-611).

During an inflammatory response, peripheral blood leukocytes are recruited to the site of inflammation or injury by a series of specific cellular interactions. The lymphocyte function associated antigen-1 (LFA-1) has been identified as the major integrin that mediates lymphocyte adhesion and activation leading to a normal immune response, as well as several pathological states (Springer, T. A., Nature 346: 425-434 (1990)). The binding of LFA-1 to ICAMs mediate a range of lymphocyte functions including lymphokine production of helper T-cells in response to antigen presenting cells, T-lymphocyte mediated target cells lysis, natural killing of tumor cells, and immunoglobulin production through T-cell-B-cell interactions. Thus, many facets of lymphocyte function involve the interaction of the LFA-1 integrin and its ICAM ligands. These LFA-1:ICAM mediated interactions have been directly implicated in numerous inflammatory disease states including a number of gastrointestinal inflammatory conditions, such as inflammatory bowel syndrome, colitis, celiac disease, gastritis.

Thus, there exists a need for developing methods to reduce, prevent or treat inflammatory disorders of the gastrointestinal system by antagonizing LFA-1. The present invention satisfies these needs and provides related advantages as well.

### SUMMARY OF THE INVENTION

In one aspect, a pharmaceutical formulation is provided comprising an LFA-1 antagonist or a pharmaceutically acceptable salt or ester thereof, and an excipient suitable for oral administration, wherein the LFA-1 antagonist has a systemic clearance rate greater than about 2 mL/min/kg when administered to a subject. In one embodiment, the LFA-1 antagonist can achieve a local tissue concentration of greater than about 1 μM within about 4 hours following administration to a subject. In another embodiment, the local tissue concentration of the LFA-1 antagonist is maintained at a concentration of greater than about 10 nM for at least about 8 hours following administration to a subject.

In another aspect, a method for treatment of an inflammatory or immune related disorder of one or more tissues of the gastrointestinal system in a subject is provided comprising administering to the subject in need thereof, a formulation comprising an LFA-1 antagonist or a pharmaceutically acceptable salt or ester thereof, and a pharmaceutically acceptable excipient, wherein the LFA-1 antagonist has a systemic clearance rate greater than about 2 mL/min/kg when administered to a subject. In one embodiment, following administration, the LFA-1 antagonist is present in a therapeutically effective concentration within about 1 mm of an epithelial surface to which the formulation is delivered and is present in blood plasma below a therapeutically effective level, within about 4 hours following administration. In another embodiment, the LFA-1 antagonist has a local tissue concentration of greater than about 10 nM within about 4 hours following administration to the subject. In some other embodiments, the LFA-1 antagonist has a local tissue concentration of greater than about 1 μM and a systemic concentration as measured in plasma of less than about 100 nM, within about 4 hours following administration to the subject. In yet other embodiments, the LFA-1 antagonist maintains the local tissue concentration of greater than about 10 nM for at least about 8 hours following administration to a subject. In various embodiments, the local tissue concentration of the LFA-1 antagonist is within about 1 mm of an epithelial surface to which the formulation is applied.

In some embodiments, the LFA-1 antagonist is a directly competitive antagonist. In other embodiments, the LFA-1 antagonist can inhibit T-cell attachment to ICAM-1 by about 50% or more at a concentration of about 100 nM.

In various embodiments, the LFA-1 antagonist is a compound of Formula I or II and/or its pharmaceutically acceptable salts or esters, having the following structures:

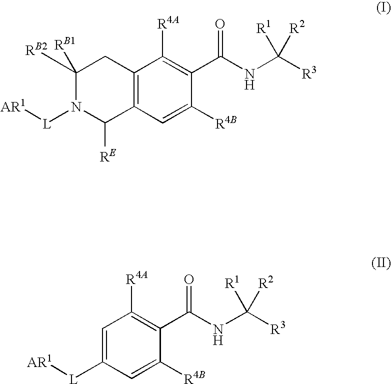

Wherein R¹ and R² can each be independently hydrogen, an amino acid side chain, —(CH₂)ₘOH, —(CH₂)ₘaryl, —(CH₂)ₘheteroaryl, wherein m is 0-6, —CH(R^{1A})(OR^{1B}), —CH(R^{1A})(NHR^{1B}), U-T-Q, or an aliphatic, alicyclic, heteroaliphatic or heteroalicyclic moiety optionally substituted with U-T-Q,

wherein U is absent, —O—, —S(O)₀₋₂—, —SO₂N(R^{1A}), —N(R^{1A}), —N(R^{1A})C(═O)—, —N(R^{1A})C(═O)—O—, —N(R^{1A})C(═O)—N(R^{1B})—, —N(R^{1A})—SO₂—, —C(═O)—, —C(═O)—O—, —O—C(═O)—, aryl, heteroaryl, alkylaryl, alkylheteroaryl, —C(═O)—N(R^{1A})—, —OC(═O)N(R^{1A})—, —C(═N—R^{1E})—, —C(═N—R^{1E})—O—, —C(═N—R^{1E})—N(R^{1A})—, —O—C(═N—R^{1E})—N(R^{1A})—, —N(R^{1A})C(═N—R^{1E})—, —N(R^{1A})C(═N—R^{1E})—O—, —N(R^{1A})C(═N—R^{1E})—N(R^{1B})—, —P(═O)(OR^{1A})—O—, or —P(═O)(R^{1A})—O—;
T is absent, an aliphatic, heteroaliphatic, aryl, heteroaryl, alkylaryl or alkylheteroaryl moiety; and
Q is hydrogen, halogen, cyano, isocyanate, —OR^{1B}; —SR^{1B}; —N(R^{1B})₂, —NHC(═O)OR^{1B}, —NHC(═O)N(R^{1B})₂, —NHC (═O)R^{1B}, —NHSO₂R^{1B}, —NHSO₂N(R^{1B})₂, —NHSO₂NHC(═O)OR^{1B}, —NHC(═O)NHSO₂R^{1B}, —C(═O)NHC(═O)OR^{1B}, —C(═O)NHC(═O)R^{1B}, —C(═O)NHC(═O)N(R^{1B})₂, —C(═O)NHSO₂R^{1B}, —C(═O)NHSO₂N(R^{1B})₂, —C(═S)N(R^{1B})₂, —SO₂R^{1B}, SO₂OR^{1B}, SO₂N(R^{1B})₂, —SO₂NHC(═O)R^{1B}, —OC(═O)—N(R^{1B})₂, —OC(═O)R^{1B}, —OC(═O)NHC(═O)R^{1B}, —OC(═O)NHSO₂R^{1B}, —OSO₂R^{1B}, or an aliphatic heteroaliphatic, aryl or heteroaryl moiety, or wherein R¹ and R² taken together can be an alicyclic or heterocyclic moiety, or together can be

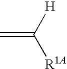

wherein each occurrence of R^{1A} and R^{1B} is independently hydrogen, an aliphatic, alicyclic, heteroaliphatic, heterocyclic, aryl, heteroaryl, alkylaryl or alkylheteroaryl moiety, —C(═O)R^{1C}, or —C(═O)NR^{1C}R^{1D}; wherein each occurrence of R^{1C} and R^{1D} is independently hydrogen, hydroxyl, or an aliphatic, heteroaliphatic, aryl, heteroaryl, alkylaryl or alkylheteroaryl moiety; and R^{1E} is hydrogen, an aliphatic, alicyclic, heteroaliphatic, heterocyclic, aryl, heteroaryl, alkylaryl or alkylheteroaryl moiety, —CN, —OR^{1C}, —NR^{1C}R^{1D} or —SO₂R^{1C};
R³ is —C(═O)OR^{3A}, —C(═O)H, CH₂OR^{3A}, —CH₂C(═O)-alkyl, —C(═O)NH(R^{3A}), —CH₂X⁰; wherein each occurrence of R^{3A} is independently hydrogen, a protecting group, an aliphatic, alicyclic, heteroaliphatic, heteroalicyclic, aryl, heteroaryl, alkylaryl, alkylheteroaryl, heteroalkylaryl heteroalkylheteroaryl moiety, or pharmaceutically acceptable salt or ester, or R^{3A}, taken together with R¹ and R², forms a heterocyclic moiety; wherein X⁰ is a halogen selected from F, Br or I;
wherein R^{4A} and R^{4B} is independently a halogen selected from F, Cl, Br or I; and R^{B1}, R^{B2} and R^{E} is independently hydrogen or substituted or unsubstituted lower alkyl;
AR¹ is a monocyclic or polycyclic aryl, heteroaryl, alkylaryl, alkylheteroaryl, alicyclic or heterocyclic moiety; and,
L is absent or is V-W-X-Y-Z, wherein each occurrence of V, W, X, Y and Z is independently absent, C═O, NR^{L1}, —O—, —C(R^{L1})═, ═C(R^{L1})—, —C(R^{L1})(R^{L2}), C(═NR^{L1}), C(═NR^{L1}), —N═, S(O)₀₋₂; a substituted or unsubstituted C₁₋₆ alkenylidene or C₂₋₆ alkenylidine chain wherein up to two non-adjacent methylene units is independently optionally replaced by —C(═O)—, —CO₂—, —C(═O)C(═O)—, —C(C═O)NR^{L3}—, —OC(═O)—, —OC(═O)NR^{L3}—, NR^{L3}NR^{L4}, —NR^{L3}NR^{L4}C(═O)—, —NR^{L3}C(═O)—, NR^{L3}CO₂—, NR^{L3}C(═O)NR^{L4}—, —S(═O)—, —SO₂—, —NR^{L3}SO₂—, —SO₂NR^{L3}, —NR^{L3}SO₂NR^{L4}, —O—, —S—, or —NR^{L3}—; wherein each occurrence of R^{L3} and R^{L4} is independently hydrogen, alkyl, heteroalkyl, aryl, heteroaryl or acyl; or an aliphatic, alicyclic, heteroaliphatic, heteroalicyclic, aryl, heteroaryl, alkylaryl or alkylheteroaryl moiety; and each occurrence of R^{L1} and R^{L2} is independently hydrogen, hydroxyl, protected hydroxyl, amino, protected amino, thio, protected thio, halogen, cyano, isocyanate, carboxy, carboxyalkyl, formyl, formyloxy, azido, nitro, ureido, thioureido, thiocyanato, alkoxy, aryloxy, mercapto, sulfonamido, benzamido, tosyl, or an aliphatic, alicyclic, heteroaliphatic, heteroalicyclic, aryl, heteroaryl, alkylaryl or alkylheteroaryl moiety, or wherein one or more occurrences of R^{L1} and R^{L2}, taken together, or taken together with one of V, W, X, Y or Z form an alicyclic or heterocyclic moiety or form an aryl or heteroaryl moiety.

In another embodiment, the LFA-1 antagonist has one of the following formulae:

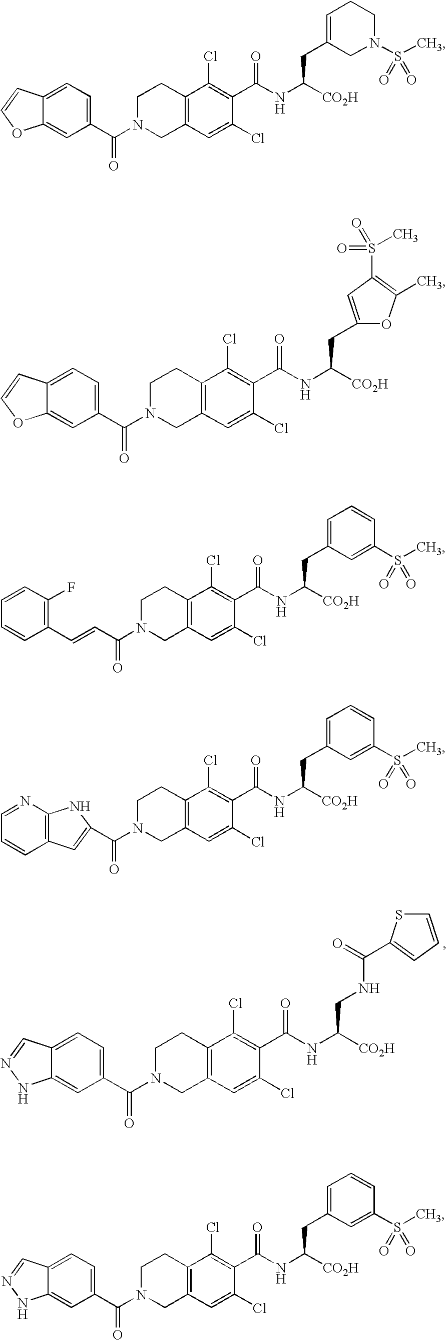

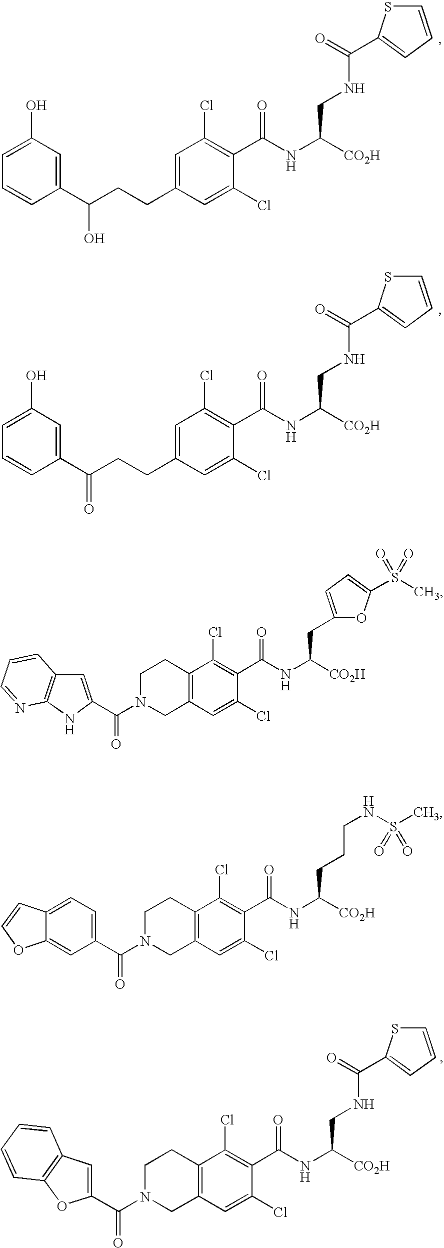

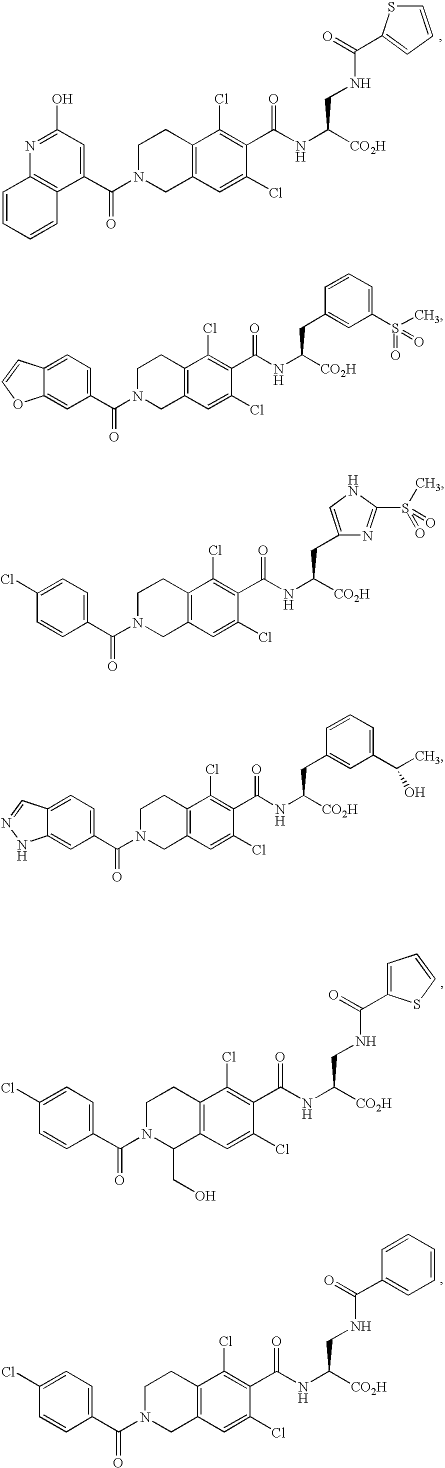

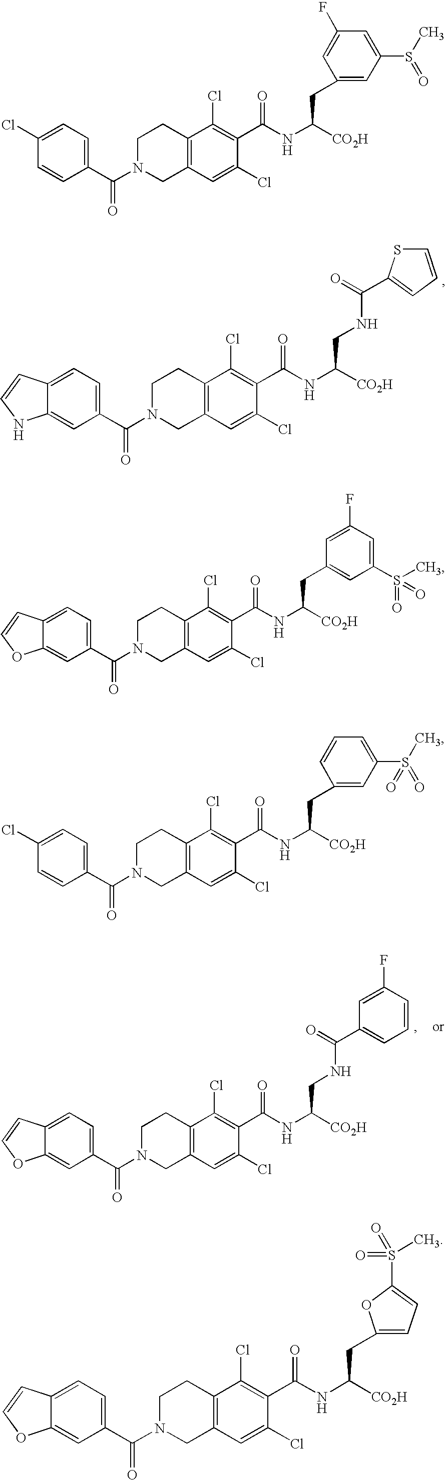

In some embodiments, the LFA-1 antagonist is a compound having the following formula:

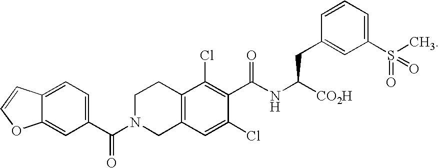

In another embodiment, the LFA-1 antagonist is any of crystalline Forms A, B, C, D, or E, the amorphous form or a combination thereof, of the compound having the following formula:

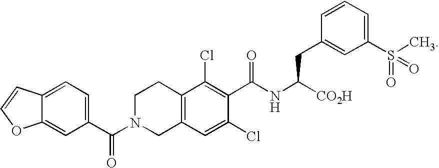

In one embodiment, the LFA-1 antagonist is Form A of a compound having the following formula:

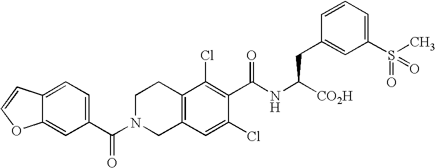

In yet other embodiments, the LFA-1 antagonist is a sodium, potassium, lithium, magnesium, zinc, or calcium salt.

In some embodiments, the formulation is in the form of a tablet, capsule, suspension, powder, crystalline forms, suppository, microparticle, or nanoparticle. In other embodiments, the excipient is water, buffered aqueous solution, surfactant, volatile liquid, starch, polyol, granulating agent, microcrystalline cellulose, diluent, lubricant, acid, base, salt, emulsion, oil, wetting agent, chelating agent, antioxidant, sterile solution, complexing agent or disintegrating agent. In various embodiments, the surfactant is oleic acid, cetylpyridinium chloride, soya lecithin, polyoxyethylene sorbitan monolaurate, polyoxyethylene sorbitan monostearate, polyoxyethylene sorbitan monooleate, polyoxyethylene stearyl ether, polyoxyethylene oleyl ether, polyoxyethylene-polyoxypropylene-ethylenediamine block copolymer, polyoxypropylene-polyoxyethylene block copolymer or castor oil ethoxylate.

The invention also provides formulations further comprising a topical penetration enhancer. In one embodiment, the topical penetration enhancer is a sulfoxide, ether, surfactant, alcohol, fatty acid, fatty acid ester, polyol, amide, terpene, alkanone or organic acid. In other embodiments, the formulation can include at least one additional therapeutic agent which is a 5-aminosalicylates (5-ASA) compound, corticosteroid, antibiotic, calcineurin inhibitor, or immunomodulator. In one embodiment, the 5-ASA compound is sulfasalazine, osalazine, or mesalamine. In other embodiments, the corticosteroid is prednisone or budesonide. In yet other embodiments, the antibiotic is metronidazole or ciprofloxacin. In one embodiment, the immunomodulator is 6-mercaptopurine, azathioprine, methotrexate, infliximab, or adalimumab. In further embodiments, the calcineurin inhibitor is cyclosporine, tacrolimus, pimecrolimus, or sirolimus.

In other embodiments, the method includes administering to the subject an additional therapeutic agent. In various embodiments, the administering the additional therapeutic agent is concurrent with, prior to, or subsequent to administering the LFA-1 antagonist therapeutic agent or a pharmaceutically acceptable salt or ester thereof. In yet other embodiments, the additional therapeutic agent is an antioxidant, antiinflammatory agent, antimicrobial agent, antiangiogenic agent, or anti-apoptotic agent. In some embodiments, the additional therapeutic agent is a 5-aminosalicylates (5-ASA) compound, corticosteroid, antibiotic, calcineurin inhibitor, or immunomodulator. In some embodiments, the 5-ASA compound is sulfasalzine, osalazine, or mesalamine. In some other embodiments, the corticosteroid is prednisone or budesonide. In yet other embodiments, the antibiotic is metronidazole or ciprofloxacin. In further embodiments, the immunomodulator is 6-mercaptopurine, azathioprine, methotrexate, infliximab, or adalimumab. In other embodiments, the calcineurin inhibitor is cyclosporine, tacrolimus, pimecrolimus, or sirolimus.

In various embodiments, the localized inflammatory or immune related disorder is inflammatory bowel disease, Crohn's disease, ulcerative colitis, or oral lichen planus.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the results of a lymphocyte adhesion inhibition assay and IL-2 release assay. For the inhibition assay, EC50 values were calculated for inhibition of binding between HuT78 or Jurkat T-cells and immobilized ICAM-1. For the IL-2 release assay, EC50 values were calculated for inhibition of IL-2 production from peripheral blood mononuclear cells following the addition of staph enterotoxin B antigen. This was done in the presence of 10% human serum.

FIG. 2 is a graphical representation of histopathological evaluation of biopsies taken before and after treatment of a dog eye with Compound 12.

FIG. 3 illustrates the mean change in Schirmer test score at weeks, 2, 4, 8, and 12 for eyes in dogs treated with Compound 12.

FIG. 4 illustrates percentage of dog eyes with a Schirmer test score of greater than 10 mm at 2, 4, 8, and 12 weeks with a formulation of 1% Compound 12 (TID; three times daily).

FIG. 5 illustrates percentage of eyes with a greater than 4 mm improvement in Schirmer test score at 2, 4, 12, 16, and 26 weeks for subjects treated with a formulation of 1% Compound 12 (TID) compared to literature results for 2% CsA (BID; two times daily).

FIG. 6 illustrates a timecourse of mean plasma levels of Compound 12 treatment (human) with 5% Compound 12.

FIG. 7 illustrates tear Cₘᵢₙ levels for human subjects treated with 1% Compound 12 QD (once a day).

FIG. 8 illustrates the dose/drug Cₘₐₓ tear level relationship for administration of Compound 12 in humans (QD and TID).

FIG. 9 illustrates the dose/AUC (area under the concentration-time curve) and dose/mean Cₘₐₓ (maximum observed concentration) tear level relationship for human subjects treated QD with Compound 12.

FIG. 10 is a graphical representation of a whole body autoradiograph for a male Sprague Dawley Animal 0.5 hour after a single topical ocular administration of [¹⁴C]-Compound 12 (1 mg/eye).

FIG. 11 is a graphical representation of a whole-body autoradiograph for a male Sprague Dawley Animal 2 hours after a single topical ocular administration of [¹⁴C]-Compound 12 (1 mg/eye).

FIG. 12 is a graphical representation of a whole-body autoradiograph for a male Sprague Dawley Animal 8 hours after a single topical ocular administration of [¹⁴C]-Compound 12 (1 mg/eye).

FIG. 13 is a graphical representation of a whole-body autoradiograph for a male Sprague Dawley Animal 12 hours after a single topical ocular administration of [¹⁴C]-Compound 12 (1 mg/eye).

FIG. 14 is a graphical representation of a whole-body autoradiograph for a male Sprague Dawley Animal 24 hours after a single topical ocular administration of [¹⁴C]-Compound 12 (1 mg/eye).

FIG. 15 illustrates rat ocular pharmacokinetics of [¹⁴C]-Compound 12.

FIG. 16 illustrates dog ocular pharmacokinetics of [¹⁴C]-Compound 12.

FIG. 17 is a graphical representation of the timecourse of drug plasma levels for Compound 12 following single IV doses in rats.

FIG. 18 is a graphical representation of the timecourse of drug plasma levels for Compound 12 following single IV doses in dogs.

FIG. 19 illustrates the dose/drug AUC (in tears) relationship for Compound 12 administered to dogs.

FIG. 20 illustrates the drug tear concentration profiles of Compound 12 measured after 13 weeks of TID ocular dosing in rabbits.

FIG. 21 illustrates the drug tear concentration profiles of Compound 12 measured after 13 weeks of TID ocular dosing in dogs.

FIG. 22 illustrates mean drug tear concentrations in right and left eyes of rabbits following topical instillation of a single dose of Compound 12.

FIG. 23 illustrates the drug plasma level in rats for various topical applications of Compound 12.

### DETAILED DESCRIPTION OF THE INVENTION

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a”, “an” and “the” includes plural references unless the context clearly dictates otherwise.

As used herein, “agent” or “biologically active agent” refers to a biological, pharmaceutical, or chemical compound or other moiety. Non-limiting examples include simple or complex organic or inorganic molecule, a peptide, a protein, an oligonucleotide, an antibody, an antibody derivative, antibody fragment, a vitamin derivative, a carbohydrate, a toxin, or a chemotherapeutic compound. Various compounds can be synthesized, for example, small molecules and oligomers (e.g., oligopeptides and oligonucleotides), and synthetic organic compounds based on various core structures. In addition, various natural sources can provide compounds for screening, such as plant or animal extracts, and the like. A skilled artisan can readily recognize that there is no limit as to the structural nature of the agents of the present invention.

The term “agonist” as used herein refers to a compound having the ability to initiate or enhance a biological function of a target protein, whether by inhibiting the activity or expression of the target protein. Accordingly, the term “agonist” is defined in the context of the biological role of the target polypeptide. While preferred agonists herein specifically interact with (e.g. bind to) the target, compounds that initiate or enhance a biological activity of the target polypeptide by interacting with other members of the signal transduction pathway of which the target polypeptide is a member are also specifically included within this definition.

The terms “antagonist” and “inhibitor” are used interchangeably, and they refer to a compound having the ability to inhibit a biological function of a target protein, whether by inhibiting the activity or expression of the target protein. Accordingly, the terms “antagonist” and “inhibitors” are defined in the context of the biological role of the target protein. While preferred antagonists herein specifically interact with (e.g. bind to) the target, compounds that inhibit a biological activity of the target protein by interacting with other members of the signal transduction pathway of which the target protein is a member are also specifically included within this definition. A preferred biological activity inhibited by an antagonist of LFA-1, for example, is associated with an undesired inflammatory or immune response as manifested in inflammatory or autoimmune disease, respectively.

A “directly competitive inhibitor” or “directly competitive antagonist” refers to a ligand, which includes biomolecules, peptides, and synthetic small organic molecules, which binds directly to the active site of the biological target molecule, and directly prevents a substrate from binding to it. For example, a directly competitive inhibitor of the interaction of LFA-1 and ICAM-1, binds to LFA-1 at the site where ICAM-1 binds, and thus directly prevents ICAM-1 from binding.

“Allosteric inhibitor” as used herein refers to a ligand which includes biomolecules, peptides, and synthetic small organic molecules, that binds to a biological target molecule at a site other than the binding site of the interaction which is being inhibited. The interaction changes the shape of the biological target molecule so as to disrupt the usual complex between the biological target molecule and its substrate. This results in inhibition of the normal activity of such complex formation. For example, an allosteric inhibitor of the interaction of LFA-1 and ICAM-1, binds to LFA-1 at a site other than that where ICAM-1 binds, but it disrupts the binding site of ICAM-1 such that the interaction of LFA-1 and ICAM-1 is reduced.

The term “selective inhibition” or “selectively inhibit” as applied to a biologically active agent refers to the agent's ability to selectively reduce the target signaling activity as compared to off-target signaling activity, via direct or interact interaction with the target.

“Th1” and “Th2” as used herein refer to helper T cells which are found in two distinct cell types, Th1 and Th2, distinguished by the cytokines they produce and respond to and the immune responses they are involved in. Th1 cells produce pro-inflammatory cytokines like IFN-g, TNF-b and IL-2, while Th2 cells produce the cytokines IL-4, IL-5, IL-6 and IL-13.

An “anti-cancer agent”, “anti-tumor agent” or “chemotherapeutic agent” refers to any agent useful in the treatment of a neoplastic condition. One class of anti-cancer agents comprises chemotherapeutic agents. “Chemotherapy” means the administration of one or more chemotherapeutic drugs and/or other agents to a cancer patient by various methods, including intravenous, oral, intramuscular, intraperitoneal, intravesical, subcutaneous, transdermal, buccal, or inhalation or in the form of a suppository.

The term “cell proliferation” refers to a phenomenon by which the cell number has changed as a result of division. This term also encompasses cell growth by which the cell morphology has changed (e.g., increased in size) consistent with a proliferative signal.

The term “co-administration,” “administered in combination with,” and their grammatical equivalents, as used herein, encompasses administration of two or more agents to an animal so that both agents and/or their metabolites are present in the animal at the same time. Co-administration includes simultaneous administration in separate compositions, administration at different times in separate compositions, or administration in a composition in which both agents are present.

The term “effective amount” or “therapeutically effective amount” refers to that amount of a compound described herein that is sufficient to effect the intended application including but not limited to disease treatment, as defined below. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g. reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried.

As used herein, “treatment” or “treating,” or “palliating” or “ameliorating” are used interchangeably herein. These terms refers to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. The compositions may be administered to a subject to prevent progression of physiological symptoms or to prevent progression of the underlying disorder

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

As used herein, the term “pharmaceutically acceptable salt” refers to those salts which are suitable for pharmaceutical use, preferably for use in the tissues of humans and lower animals without undue irritation, allergic response and the like. Pharmaceutically acceptable salts of amines, carboxylic acids, and other types of compounds, are well known in the art. For example, S. M. Berge, et al., describe pharmaceutically acceptable salts in detail in J Pharmaceutical Sciences, 66: 1-19 (1977), incorporated herein by reference. The salts can be prepared in situ during the final isolation and purification of the compounds of the invention, or separately by reacting a free base or free acid function with a suitable reagent, as described generally below. For example, a free base function can be reacted with a suitable acid. Furthermore, where the compounds of the invention carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzoate, bisulfate, borate, butyrate, camphorate, camphorsulfonate, citrate, cyclopentanepropionate, digluconate, dodecylsulfate, formate, fumarate, glucoheptonate, glycerophosphate, gluconate, hemisulfate, heptanoate, hexanoate, -hydroiodide, 2-hydroxy-ethanesulfonate, lactobionate, lactate, laurate, lauryl sulfate, malate, maleate, malonate, methanesulfonate, nicotinate, nitrate, oleate, oxalate, palmitate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed by direct reaction with the drug carboxylic acid or by using counterions such as halide, hydroxide, carboxylate, sulfate, phosphate, nitrate, sulfonate and aryl sulfonate.

“Pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions of the invention is contemplated. Supplementary active ingredients can also be incorporated into the compositions.

“Prodrug” is meant to indicate a compound that may be converted under physiological conditions or by solvolysis to a biologically active compound described herein. Thus, the term “prodrug” refers to a precursor of a biologically active compound that is pharmaceutically acceptable. A prodrug may be inactive when administered to a subject, i.e. an ester, but is converted in vivo to an active compound, for example, by hydrolysis to the free carboxylic acid. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like.

“Localized treatment” as used herein refers to treatment of an immune or inflammatory disorder wherein the drug is delivered locally and is not delivered via systemic delivery. This may include many different local areas or a few different local areas within, for example, the gastrointestinal tract to which drug is delivered to the gastrointestinal mucosa from within the lumen of the GI tract. Another example is treatment of skin, wherein the drug may be applied to many different locations or a few different locations on the skin, and wherein drug is delivered to tissues within and adjacent to the skin by absorption through the skin. Alternatively, drug may be delivered via suppository to anal mucosa and absorbed through the epithelial surfaces to tissue within and adjacent to the mucosa of the lower GI tract.

“Local delivery” as used herein refers to drug compound being carried to the site of therapeutic use. It includes, for example, applying a formulation directly to area of skin that is being treated, spraying a formulation to an area of skin being treated, spraying or inhaling a formulation intranasally to administer drug to the nasal passages, or instilling eye drops to an eye to treat the eye. In the present invention, “local delivery” also encompasses orally or nasally administering a formulation which is carried to the gastrointestinal tract, wherein the drug is brought in contact with the gastrointestinal mucosa, where the drug is absorbed into the surrounding tissue and exerts a therapeutic effect, without being directly delivered to that site from the blood circulatory system.

“Local tissue concentration” as used herein, refers to the concentration of LFA-1 antagonist within the tissue area to which the LFA-1 antagonist has been delivered and absorbed.

“Subject” refers to an animal, such as a mammal, for example a human. The methods described herein can be useful in both human therapeutics and veterinary applications. In some embodiments, the patient is a mammal, and in some embodiments, the patient is human.

The term “in vivo” refers to an event that takes place in a subject's body.

The term “in vitro” refers to an event that takes places outside of a subject's body. For example, an in vitro assay encompasses any assay run outside of a subject assay. In vitro assays encompass cell-based assays in which cells alive or dead are employed. In vitro assays also encompass a cell-free assay in which no intact cells are employed.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C— or ¹⁴C-enriched carbon are within the scope of this invention.

The compounds of the present invention may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are encompassed within the scope of the present invention.

When ranges are used herein for physical properties, such as molecular weight, or chemical properties, such as chemical formulae, all combinations and subcombinations of ranges and specific embodiments therein are intended to be included. The term, “about” when referring to a number or a numerical range means that the number or numerical range referred to is an approximation within experimental variability (or within statistical experimental error), and thus the number or numerical range may vary from, for example, between 1% and 15% of the stated number or numerical range. The term “comprising” (and related terms such as “comprise” or “comprises” or “having” or “including”) includes those embodiments, for example, an embodiment of any composition of matter, composition, method, or process, or the like, that “consist of” or “consist essentially of” the described features.

Abbreviations used herein have their conventional meaning within the chemical and biological arts.

The term “aliphatic”, as used herein, includes both saturated and unsaturated, straight chain (unbranched) or branched aliphatic hydrocarbons, which are optionally substituted with one or more functional groups. As will be appreciated by one of ordinary skill in the art, “aliphatic” is intended herein to include, but is not limited to, alkyl, alkenyl, alkynyl moieties. Thus, as used herein, the term “alkyl” includes straight and branched alkyl groups. An analogous convention applies to other generic terms such as “alkenyl”, “alkynyl” and the like.

Furthermore, as used herein, the terms “alkyl”, “alkenyl”, “alkynyl”, and the like encompass both substituted and unsubstituted groups. In certain embodiments, as used herein, “lower alkyl” is used to indicate those alkyl groups (substituted, unsubstituted, branched or unbranched) having about 1-6 carbon atoms.

In certain embodiments, the alkyl, alkenyl and alkynyl groups employed in the invention contain about 1-20 aliphatic carbon atoms. In certain other embodiments, the alkyl, alkenyl, and alkynyl groups employed in the invention contain about 1-10 aliphatic carbon atoms. In yet other embodiments, the alkyl, alkenyl, and alkynyl groups employed in the invention contain about 1-8 aliphatic carbon atoms. In still other embodiments, the alkyl, alkenyl, and alkynyl groups employed in the invention contain about 1-6 aliphatic carbon atoms. In yet other embodiments, the alkyl, alkenyl, and alkynyl groups employed in the invention contain about 1-4 carbon atoms. Illustrative aliphatic groups thus include, but are not limited to, for example, methyl, ethyl, n-propyl, isopropyl, allyl, n-butyl, sec-butyl, isobutyl, tert-butyl, n-pentyl, sec-pentyl, isopentyl, tert-pentyl, n-hexyl, sec-hexyl, moieties and the like, which again, may bear one or more substituents.

Alkenyl groups include, but are not limited to, for example, ethenyl, propenyl, butenyl, and the like. Representative alkynyl groups include, but are not limited to, ethynyl, 2-propynyl and the like.

The term “lower alkylene” as used herein refers to a hydrocarbon chain which links together two other groups, i.e. is bonded to another group at either end, for example methylene, ethylene, butylene and the like. Such a substituent is preferably from 1 to 10 carbons and more preferably from 1 to 5 carbons. Such groups may be substituted, preferably with an amino, acetylamino (a lower alkylcarbonyl group bonded via a nitrogen atom), or cyclo lower alkyl group. By the latter is meant a saturated hydrocarbon ring, preferably with a total of 3 to 10 methylenes (inclusive of the attachment carbons), more preferably 3 to 6.

The term “alicyclic”, as used herein, refers to compounds which combine the properties of aliphatic and cyclic compounds and include but are not limited to monocyclic, or polycyclic aliphatic hydrocarbons and bridged cycloalkyl compounds, which are optionally substituted with one or more functional groups.

As will be appreciated by one of ordinary skill in the art, “alicyclic” is intended herein to include, but is not limited to, cycloalkyl, cycloalkenyl, and cycloalkynyl moieties, which are optionally substituted with one or more functional groups.

Illustrative alicyclic groups thus include, but are not limited to, for example, cyclopropyl, —CH₂-cyclopropyl, cyclobutyl, —CH₂-cyclobutyl, cyclopentyl, —CH₂-cyclopentyl, cyclohexyl, —CH₂-cyclohexyl, cyclohexenylethyl, cyclohexanylethyl, norbornyl moieties and the like, which again, may bear one or more substituents.

The term “alkoxy” or “alkyloxy”, as used herein refers to a saturated or unsaturated parent molecular moiety through an oxygen atom. In certain embodiments, the alkyl group contains about 1-20 aliphatic carbon atoms. In certain other embodiments, the alkyl group contains about 1-10 aliphatic carbon atoms. In yet other embodiments, the alkyl group employed in the invention contains about 1-8 aliphatic carbon atoms. In still other embodiments, the alkyl group contains about 1-6 aliphatic carbon atoms. In yet other embodiments, the alkyl group contains about 1-4 aliphatic carbon atoms. Examples of alkoxy, include but are not limited to, methoxy, ethoxy, isopropoxy, n-butoxy, i-butoxy, sec-butoxy, tert-butoxy, neopentoxy, n-hexloxy and the like.

The term “lower alkoxy” as used herein refers to a lower alkyl as defined above which may be branched or unbranched as also defined above and which is bonded by an oxygen to another group (i.e. alkyl ethers).

The term “alkylamino” refers to a group having the structure —NHR′ wherein R′ is alkyl, as defined herein. The term “aminoalkyl” refers to a group having the structure NH₂R′—, wherein as defined herein. In certain embodiments, the alkyl group contains about 1-20 aliphatic carbon atoms. In certain other embodiments, the alkyl group contains about 1-10 aliphatic carbon atoms. In yet other embodiments, the alkyl group employed in the invention contains about aliphatic carbon atoms. In still other embodiments, the alkyl group contains about 1-6 aliphatic carbon atoms. In yet other embodiments, the alkyl group contains about 1-4 aliphatic carbon atoms. Examples of alkylamino include, but are not limited to, methylamino, and the like.

Some examples of substituents of the above-described aliphatic (and other) moieties of compounds of the invention include, but are not limited to aliphatic; alicyclic; heteroaliphatic; heterocyclic; aromatic; heteroaromatic; aryl heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; Rₓ, independently includes, but is not limited to, aliphatic, alicyclic, heteroaliphatic, heterocyclic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aryl or heteroaryl substituents described above and herein may be substituted or unsubstituted. Additional examples of generally applicable substituents are illustrated by the specific embodiments shown in the Examples that are described herein.

In general, the term “aromatic moiety”, as used herein, refers to a stable mono- or polycyclic, unsaturated moiety having preferably 3-14 carbon atoms, each of which may be substituted or unsubstituted. In certain embodiments, the term “aromatic moiety” refers to a planar ring having p-orbitals perpendicular to the plane of the ring at each ring atom and satisfying the Huckel rule where the number of pi electrons in the ring is (4n+2) wherein n is an integer. A mono- or polycyclic, unsaturated moiety that does not satisfy one or all of these criteria for aromaticity is defined herein as “non-aromatic”, and is encompassed by the term “alicyclic”.

In general, the term “heteroaromatic moiety”, as used herein, refers to a stable mono- or polycyclic, unsaturated moiety having preferably 3-14 carbon atoms, each of which may be substituted or unsubstituted; and comprising at least one heteroatom selected from O, S, and N within the ring in place of a ring carbon atom). In certain embodiments, the term “heteroaromatic moiety” refers to a planar ring comprising at least one heteroatom, having p-orbitals perpendicular to the plane of the ring at each ring atom, and satisfying the Huckel rule where the number of pi electrons in the ring is (4n+2) wherein n is an integer.

It will also be appreciated that aromatic and heteroaromatic moieties, as defined herein may be attached via an alkyl or heteroalkyl moiety and thus also include—(alkyl) aromatic, -(heteroalkyl) aromatic, -(heteroalkyl) heteroaromatic, and -(heteroalkyl) heteroaromatic moieties. Thus, as used herein, the phrases “aromatic or heteroaromatic moieties” and “aromatic, (heteroalkyl) aromatic, -(heteroalkyl) heteroaromatic, and (heteroalkyl) heteroaromatic” are interchangeable. Substituents include, but are not limited to, any of the previously mentioned substituents, e.g, the substituents recited for aliphatic moieties, or for other moieties as disclosed herein, resulting in the formation of a stable compound.

The term “aryl”, as used herein, does not differ significantly from the common meaning of the term in the art, and refers to an unsaturated cyclic moiety comprising at least one aromatic ring. In certain embodiments, “aryl” refers to a mono- or bicyclic carbocyclic ring system having one or two aromatic rings including, but not limited to, phenyl, naphthyl, tetrahydronaphthyl, indanyl, indenyl and the like.

The term “heteroaryl” as used herein, does not differ significantly from the common meaning of the term in the art, and refers to a cyclic aromatic radical having from five to ten ring atoms of which one ring atom is selected from S, and N; zero, one or two ring atoms are additional heteroatoms independently selected from S, and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

It will be appreciated that aryl and heteroaryl groups (including bicyclic aryl groups) can be unsubstituted or substituted, wherein substitution includes replacement of one or more of the hydrogen atoms thereon independently with any one or more of the following moieties including, but not limited to: aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(═O)Rₓ; —C(═O)N(Rₓ)₂; —OC(═O)Rₓ; —OCO₂Rₓ; —OC(═O)N(Rₓ)₂; —N(Rₓ)₂; —S(O)₂Rₓ; —NRₓ(CO)Rₓ wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl, heteroaryl, -(alkyl) aryl or -(alkyl) heteroaryl substituents described above and herein may be substituted or unsubstituted. Additionally, it will be appreciated, that any two adjacent groups taken together may represent a 4, 5, 6, or 7-membered substituted or unsubstituted alicyclic or heterocyclic moiety. Additional examples of generally applicable substituents are illustrated by the specific embodiments shown in the Examples that are described herein.

The term “cycloalkyl”, as used herein, refers specifically to groups having three to seven, preferably three to ten carbon atoms. Suitable cycloalkyls include, but are not limited to cyclopropyl, cyclobutyl, cyclopentyl, cyclohexyl, cycloheptyl and the like, which, as in the case of aliphatic, alicyclic, heteroaliphatic or heterocyclic moieties, may optionally be substituted with substituents including, but not limited to aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(═O)Rₓ; —C(═O)N(Rₓ)₂; —OC(═O)Rₓ; —OCO₂Rₓ; —OC(═O)N(Rₓ)₂; —N(Rₓ)₂; —S(O)₂Rₓ; —NRₓ(CO)Rₓ, wherein each occurrence of Rₓ, independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl or heteroaryl substituents described above and herein may be substituted or unsubstituted. Additional examples of generally applicable substituents are illustrated by the specific embodiments shown in the Examples that are described herein.

The term “heteroaliphatic”, as used herein, refers to aliphatic moieties in which one or more carbon atoms in the main chain have been substituted with a heteroatom. Thus, a heteroaliphatic group refers to an aliphatic chain which contains one or more oxygen, sulfur, nitrogen, phosphorus or silicon atoms, e. place of carbon atoms. Heteroaliphatic moieties may be linear or branched, and saturated or unsaturated. In certain embodiments, heteroaliphatic moieties are substituted by independent replacement of one or more of the hydrogen atoms thereon with one or more moieties including, but not limited to aliphatic; alicyclic; heteroaliphatic; heterocyclic; aromatic; heteroaromatic; aryl; heteroaryl; alkylaryl; alkylheteroaryl; alkoxy; aryloxy; heteroalkoxy; heteroaryloxy; alkylthio; arylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(═O)Rₓ; —C(═O)N(Rₓ)₂; —OC(═O)R—; —OCO₂Rₓ; —OC(═O)N(Rₓ)₂; —N(Rₓ)₂; —S(O)₂Rₓ; —NRₓ(CO)Rₓ, wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl or heteroaryl substituents described above and herein may be substituted or unsubstituted. Additional examples of generally applicable substituents are illustrated by the specific embodiments shown in the Examples that are described herein.

The term “heterocycloalkyl”, “heterocycle” or “heterocyclic”, as used herein, refers to compounds which combine the properties of heteroaliphatic and cyclic compounds and include, but are not limited to, saturated and unsaturated mono- or polycyclic cyclic ring systems having 5-16 atoms wherein at least one ring atom is a heteroatom selected from S and N (wherein the nitrogen and sulfur heteroatoms may be optionally be oxidized), wherein the ring systems are optionally substituted with one or more functional groups, as defined herein. In certain embodiments, the term “heterocycloalkyl”, “heterocycle” or “heterocyclic” refers to a non-aromatic 5-, 6- or 7-membered ring or a polycyclic group wherein at least one ring atom heteroatom selected from S and N (wherein the nitrogen and sulfur heteroatoms may be optionally be oxidized), including, but not limited to, a bi- or tri-cyclic group, comprising fused six-membered rings having between one and three heteroatoms independently selected from oxygen, sulfur and nitrogen, wherein (i) each 5-membered ring has 0 to 2 double bonds, each 6-membered ring has 0 to 2 double bonds and each 7-membered ring has 0 to 3 double bonds, (ii) the nitrogen and sulfur heteroatoms may be optionally be oxidized, (iii) the nitrogen heteroatom may optionally be quaternized, and (iv) any of the above heterocyclic rings may be fused to an aryl or heteroaryl ring. Representative heterocycles include, but are not limited to, heterocycles such as furanyl, pyranyl, pyrrolyl, thienyl, pyrrolidinyl, pyrazolinyl, pyrazolidinyl, imidazolinyl, imidazolidinyl, piperidinyl, piperazinyl, oxazolyl, oxazolidinyl, isooxazolyl, isoxazolidinyl, dioxazolyl, thiadiazolyl, oxadiazolyl, tetrazolyl, triazolyl, thiatriazolyl, thiadiazolyl, oxadiazolyl, morpholinyl, thiazolyl, thiazolidinyl, isothiazolyl, isothiazolidinyl, dithiazolyl, dithiazolidinyl, tetrahydrofuryl, and benzofused derivatives thereof. In certain embodiments, a “substituted heterocycle, or heterocycloalkyl or heterocyclic” group is utilized and as used herein, refers to a heterocycle, or heterocycloalkyl or heterocyclic group, as defined above, substituted by the independent replacement of one, two or three of the hydrogen atoms thereon with but are not limited to aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(═O)Rₓ; —C(═O)N(Rₓ)₂; —OC(═O)Rₓ; —OCO₂Rₓ; —OC(═O)N(Rₓ)₂; —N(Rₓ)₂; —S(O)₂Rₓ; —NRₓ(CO)Rₓ wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl or heteroaryl described above and herein may be substituted or unsubstituted. Additionally, it will be appreciated that any of the alicyclic or heterocyclic moieties described above and herein may comprise an aryl or heteroaryl moiety fused thereto.

The terms “halo” and “halogen” used herein refer to an atom selected from fluorine, chlorine, bromine and iodine.

The term “haloalkyl” denotes an alkyl group, as defined above, having one, two, or three halogen atoms attached thereto and is exemplified by such groups as chloromethyl, bromoethyl, trifluoromethyl, and the like.

The term “amino” as used herein, refers to a primary (—NH₂), secondary (—NHRₓ), tertiary (—NRₓR_{y}), or quaternary amine (—N⁺RₓR_{y}R_{z}), where R_{y} and Rₓ are independently an aliphatic, alicyclic, heteroaliphatic, heterocyclic, aromatic or heteroaromatic moiety, as defined herein. Examples of amino groups include, but are not limited to, methylamino, dimethylamino, ethylamino, diethylamino, diethylaminocarbonyl, iso-propylamino, piperidino, trimethylamino, and propylamino.

The term “acyl”, as used herein, refers to a group having the general formula —C(═O)R, where R is an aliphatic, alicyclic, heteroaliphatic, heterocyclic, aromatic or heteroaromatic moiety, as defined herein.

The term “sulfonamido” as used herein, refers to a group of the general formula —SO₂NRxRy where Rx and Ry are independently hydrogen, or an aliphatic, alicyclic, heteroaliphatic, heterocyclic, aromatic, heteroaromatic or acyl moiety, as defined herein.

The term “benzamido”, as used herein, refers to a group of the general formula PhNRx, where Rx is hydrogen, or an aliphatic, alicyclic, heteroaliphatic, heterocyclic, aromatic, heteroaromatic or acyl moiety, as defined herein.

As used herein, the terms “aliphatic”, “heteroaliphatic”, “alkyl”, “alkenyl”, “alkynyl”, “heteroalkyl”, “heteroalkenyl”, “heteroalkynyl”, and the like encompass substituted and unsubstituted, saturated and unsaturated, and linear and branched groups. Similarly, the terms, “alicyclic”, “heterocyclic”, heterocycloalkyl”, “heterocycle” and the like, encompass substituted and unsubstituted, and saturated and unsaturated groups. Additionally, the terms “cycloalkyl”, cycloalkenyl”, cycloalkynyl”, “heterocycloalkyl”, “heterocycloalkenyl”, “heterocycloalkynyl”, “aromatic”, “heteroaromatic”, “aryl”, “heteroaryl” and the like encompass both substituted and unsubstituted groups.

The term “natural amino acid” as used herein refers to any one of the common, naturally occurring L-amino acids found in naturally occurring proteins: glycine (Gly); alanine (Ala), valine (Val), leucine (Leu), isoleucine (Ile), lysine (Lys), arginine (Arg), histidine (His), proline (Pro), serine (Ser), threonine (Thr), phenylalanine (Phe), tyrosine (Tyr), tryptophan (Trp), aspartic acid (Asp), glutamic acid (Glu), asparagine (Asn), glutamine (Gln), cysteine (Cys) and methionine (Met).

The term “unnatural amino acid” as used herein refers to all amino acids which are not natural amino acids. This includes, for example, α-, β-, D-, L-amino acid residues, and compounds of the general formula:

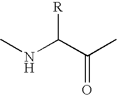

wherein the side chain R is other than the amino acid side chains occurring in nature.

More generally, the term “amino acid”, as used herein, encompasses natural amino, acids and unnatural amino acids.

The present invention provides formulated LFA-1 antagonists or pharmaceutically acceptable salts and methods of treatment of inflammatory diseases and disorders using delivery to the gastrointestinal system. The formulations can be well suited for localized treatment of the gastrointestinal mucosa, for example, by having a rapid systemic clearance rate. The formulations of the invention deliver therapeutically effective amounts of a LFA-1 antagonist locally to the gastrointestinal tissue, but systemic concentrations of the LFA-1 antagonist remain below a therapeutically effective concentration, as the clearance rate of the LFA-1 antagonist from the system is high. Advantages of localized LFA-1 antagonist therapy delivered topically include delivery of a higher concentration of active compound to the site of interest, rapid delivery of the active compound and decreased systemic effects due to lower systemic circulating levels

### Delivery of LFA-1 Antagonists to Gastrointestinal Tissues

The present invention provides formulations contain an LFA-1 antagonist as a therapeutic agent. The formulations of LFA-1 antagonists of the present invention are used for treatment of inflammatory or immune related diseases and disorders. Delivery of the formulations to the gastrointestinal (GI) system, for example organs and tissues of the mouth, throat, tongue, stomach, esophagus, small intestine (including the duodenum, jejunum, or ileum), or large intestine (including the cecum or colon), provides localized treatment by having a rapid systemic clearance rate.

LFA-1 interaction with ICAMs exerts various systemic effects throughout the body. Treatment of a disorder using an LFA-1 antagonist may result in unwanted effects due to LFA-1 antagonist activity in unwanted locations, for example, other than at the site of administration. The present invention utilizes LFA-1 antagonists which are cleared quickly from systemic circulation. By utilizing gastrointestinal delivery to the site of an inflammatory or immune disorder, unwanted systemic effects are minimized. The LFA-1 antagonists of the present invention typically have minimal systemic LFA-1 antagonist activity. In some embodiments, the LFA-1 antagonists may have undetectable systemic LFA-1 antagonist activity.

The systemic clearance rate can be calculated by various means known in the art. For example, the clearance rate for a drug may be calculated from an analysis of the drug concentration time profile for the rate of disappearance of a drug from the plasma following administration of the formulation, for example after a single intravenous injection or oral administration. The rate of disappearance may be measured by analysis of the absorption, distribution, metabolism and excretion of a radiolabelled form of a drug or other means of measuring the level of drug in plasma, such as liquid chromatography-mass spectrometry methods (LCMS), or gas chromatography or HPLC (Sapirstein et al., 1955, Am. Jour. Physiol., Vol. 181, pp. 330; U.S. Pat. No. 4,908,202). As an example, the clearance rate may be calculated by introducing the formulation to the subject by continuous intravenous infusion until an equilibrium is reached at which the plasma level of the substance (as determined by analysis of plasma samples) is steady, at which point the infusion rate is equal to the rate of clearance from plasma (Earle et al., 1946, Proc. Soc. Exp. Biol. Med., Vol. 62, pp. 262 ff.)

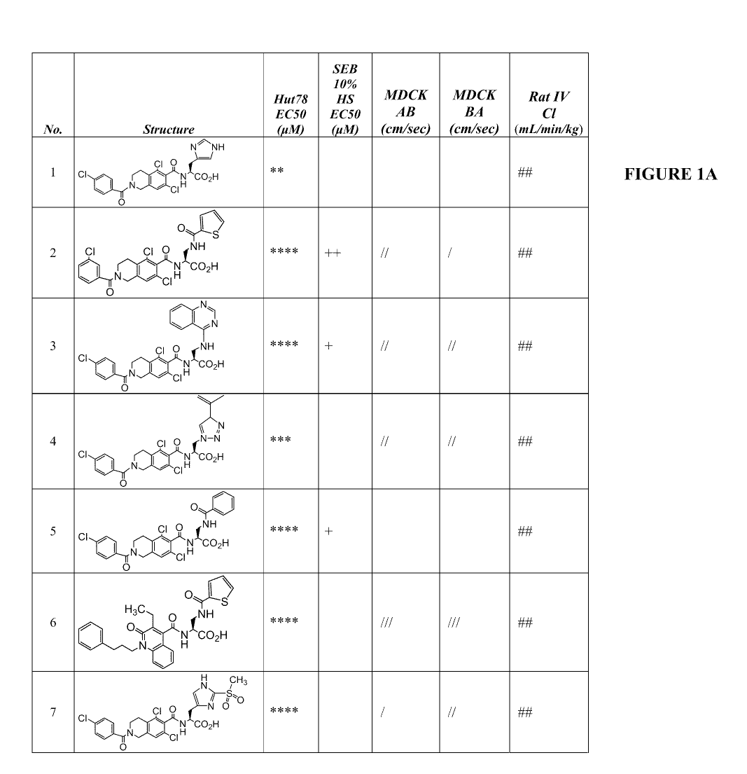

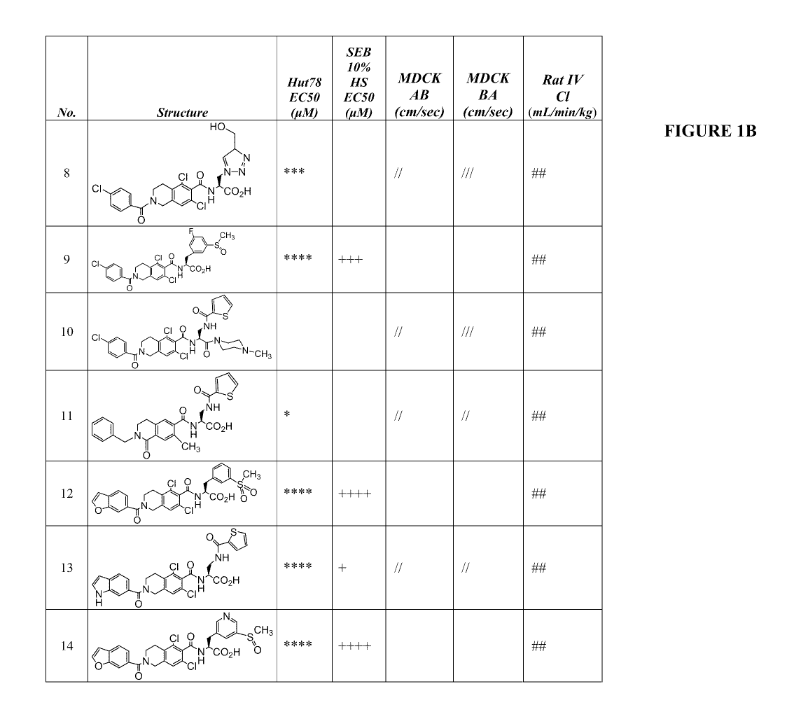

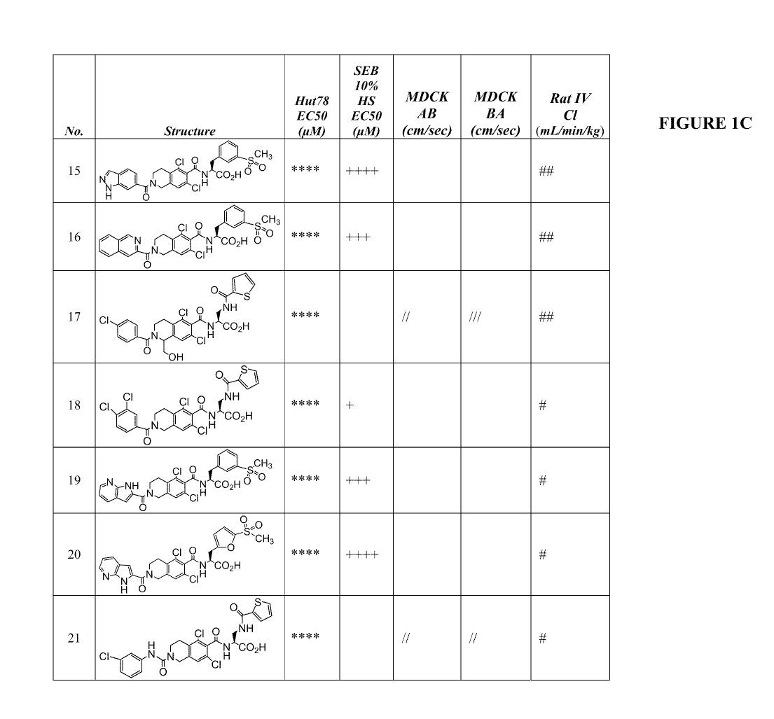

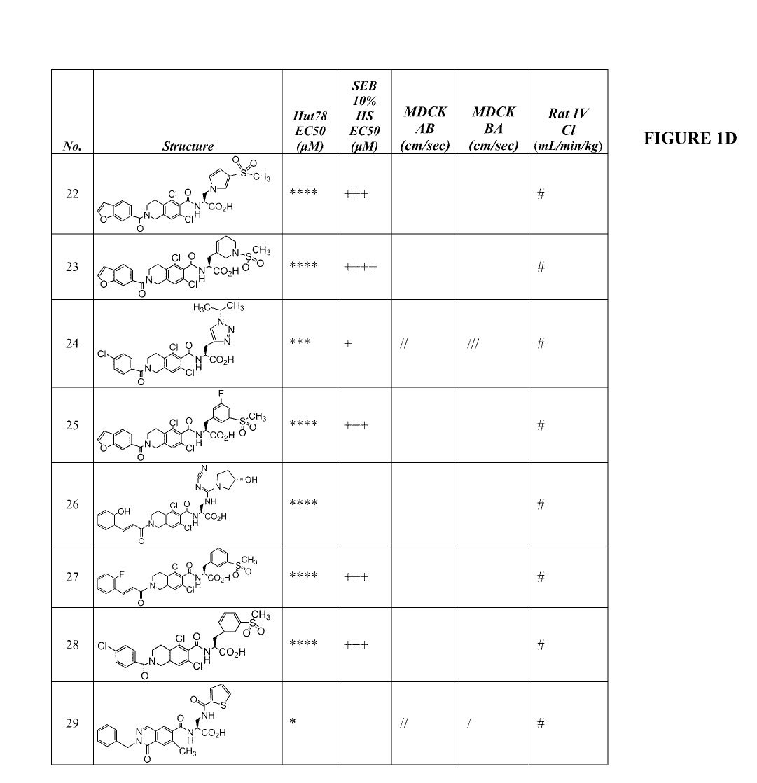

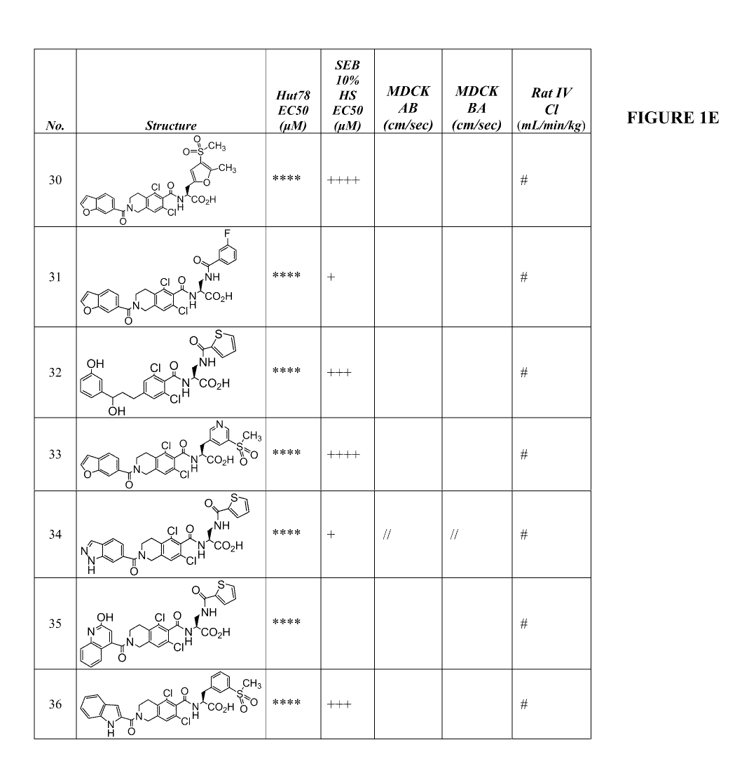

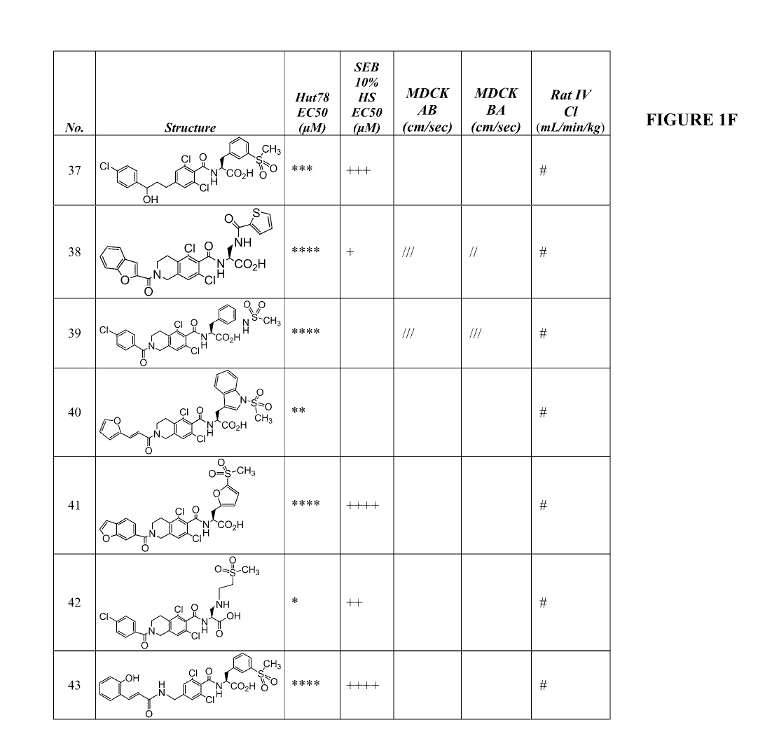

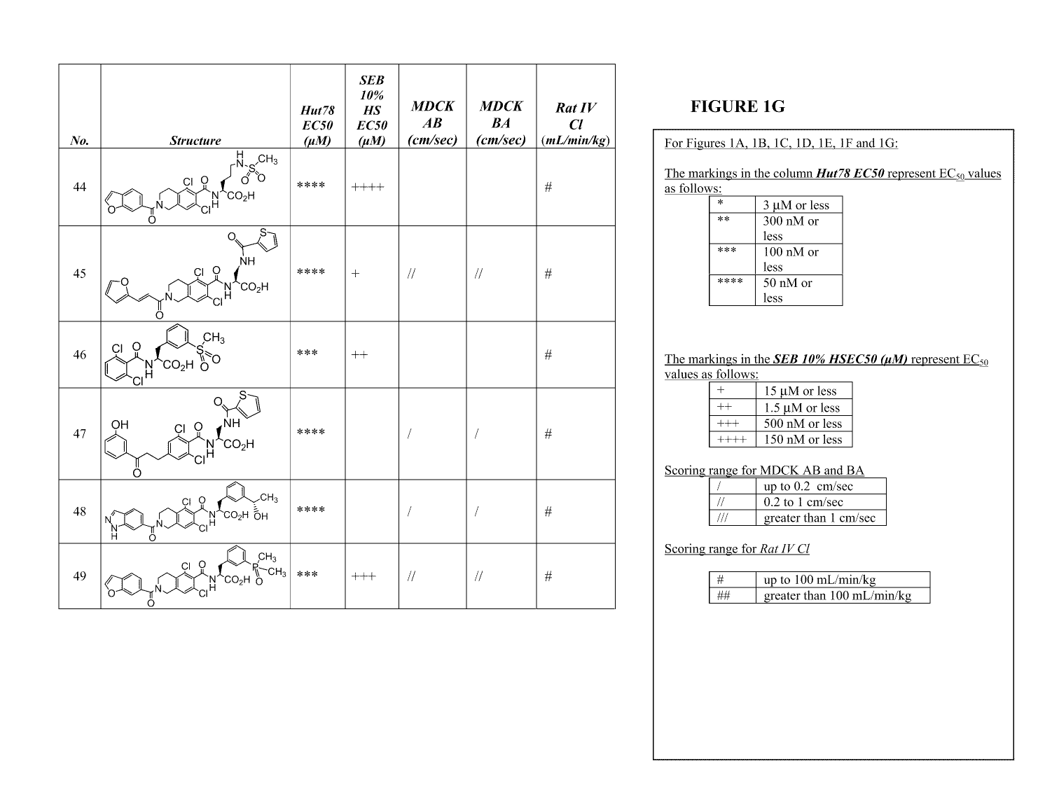

Rapid systemic clearance may be through clearance or metabolism in the liver, kidney or other organs. Data for rate of clearance through the liver in rats is given for selected compounds in FIG. 1 (see also Example 10). Where clearance occurs in a particular organ, the clearance rate is related to the blood flow to that particular organ. By knowing the mechanism in which a compound is cleared for a particular species, the clearance rate for other animals may be calculated by allometric scaling. For example, a compound of the present invention, Compound 12, is known to be cleared through the liver in rats. Based on the rate of clearance calculated in rat, the clearance of the compound may be scaled for various animals based on the known blood flow in rats compared to other animals (see Davies and Morris, “Physiological Parameters in Laboratory Animals and Humans” Pharmaceutical Research (1993) 10:1093-5). An LFA-1 antagonist of the present invention may have a systemic clearance rate approaching cardiac output, hepatic blood flow or kidney blood flow when scaled to a human. The scaling may be based on percent of cardiac output, hepatic blood flow or kidney blood. For example, 100% of rat hepatic blood flow would be approximately 55 mL/min/Kg while 100% of human hepatic blood flow would be approximately 20 mL/min/kg. In some embodiments, the compositions of the invention have a clearance rate of at least 5% of hepatic blood flow. In humans, this would mean a clearance rate of 1 mL/min/kg. In other embodiments, the LFA-1 antagonist has a clearance rate of at least about 10%, 20%, 30%, 40%, 50%, 60%, 70%, 80%, 90% or 100% of hepatic blood flow rate in humans (which would be a clearance rate in human liver of 20 mL/min/kg). In yet other embodiments, the LFA-1 antagonist has a clearance rate of at least about 110%, 120%, 130%, 140%, 150%, 175%, 200%, 220%, 240%, 260%, 280%, 300%, 320%, 340%, 360%, 380%, 400%, 420%, 440%, 460%, 480%, or 500% of hepatic blood flow rate in humans.

The clearance rates of the present invention may include clearance rates scaled to humans of approximately 1-500 mL/min/kg. In some embodiments, the LFA-1 antagonist may have a systemic clearance rate of approximately 1 mL/min/kg or greater. In other embodiments, the LFA-1 antagonist may have a systemic clearance rate of approximately 2 mL/min/kg or greater. In other embodiments, the LFA-1 antagonist may have a systemic clearance rate of approximately 3 mL/min/kg or greater. In other embodiments, the LFA-1 antagonist may have a systemic clearance rate of approximately 5 mL/min/kg or greater. In other embodiments, the LFA-1 antagonist may have a systemic clearance rate of approximately 7 mL/min/kg or greater. In some embodiments, the LFA-1 antagonist may have a systemic clearance rate of approximately 10 mL/min/kg or greater. In other embodiments, the LFA-1 antagonist may have a systemic clearance rate of approximately 15 mL/min/kg or greater. In other embodiments, the LFA-1 antagonist may have a systemic clearance rate of approximately 20 mL/min/kg or greater. In other embodiments, the LFA-1 antagonist may have a systemic clearance rate of approximately 25 mL/min/kg or greater. In some embodiments, the LFA-1 antagonist may have a systemic clearance rate of approximately 30 mL/min/kg or greater. In some embodiments, the LFA-1 antagonist may have a systemic clearance rate of approximately 40 mL/min/kg or greater. In other embodiments, the LFA-1 antagonist may have a systemic clearance rate of approximately 50 mL/min/kg or greater. In yet other embodiments, the LFA-1 antagonist may have a systemic clearance rate of at least about 60, 65, 70, 75, 80, 85, 90, 95, or 100 mL/min/kg.

In another aspect of the invention, the LFA-1 antagonist of the present invention has an inhibitory effect on LFA-1 binding to ICAM-1. The inhibitory effect of the LFA-1 antagonists of the present invention may be tested using any of a variety of known binding assays in the art, including direct cell binding to ICAM-1 coated plates, enzyme-linked immunoadsorbant assay (ELISA), radioimmunoassay (RIA) or the use of biosensors. The inhibitory effect of a drug is typically measured as an IC50 value, which measure how much of a compound is required to inhibit 50% of a biological process. For example, the LFA-1 antagonist of the present invention may inhibit a biological process such as T-cell attachment to ICAM-1, by 50% or more. Alternatively, the inhibitory effect may be calculated as an EC50 value, which measures the effective concentration by which the drug functions to achieve 50% of the desired effect. For example, the EC50 value could be measured to calculate inhibition of LFA-1 expressing T-cells from binding to ICAM-1. For example, the T-cell line HuT78 (ATCC TIB-161) may be bound to ICAM-1 coated plates in the presence of increasing concentrations of an LFA-1 antagonist. In some embodiments, the LFA-1 antagonist is a directly competitive inhibitor of the interaction between LFA-1 and ICAM-1. Examples of competitive binding experiments for LFA-1 antagonists are described in the art, for example, in U.S. Patent Application No 2005/0148588, and U.S. Provisional Application No. 60/999,571, and; the contents of which are expressly incorporated herein by reference. (See also, Gadek et al., Science 295, 1086-1089, (2002); Keating et al., Protein Science, 15, 290-303 (2006)). The EC50, or IC50, may be used in embodiments described below. Such assays can be used to identify inhibitors that are directly competitive inhibitors.

The LFA-1 antagonist can inhibit HuT78 cellular binding to ICAM-1 coated plates with an EC50 of approximately 10 μM or less. In some embodiments, the LFA-1 antagonist inhibits HuT78 cellular binding to ICAM-1 coated plates with an EC50 of approximately 1 μM or less. In other embodiments, the LFA-1 antagonist inhibits HuT78 cellular binding to ICAM-1 coated plates with an EC50 of approximately 100 nM or less. In yet other embodiments, the LFA-1 antagonist inhibits HuT78 cellular binding to ICAM-1 coated plates with an EC50 of approximately 10, 5 or 1 nM or less. Data for the inhibition of HuT78 cellular binding to ICAM-1 for selected LFA-1 antagonists of Formula I and Formula II are shown in FIG. 1.

Alternatively, the inhibitory effect of the LFA-1 antagonists of the present invention may also be tested using known downstream events following binding of LFA-1 to ICAM-1. For example, it is known that IL-2 is released from human T-cells in primary culture following stimulation by the superantigen staph enterotoxin B (SEB) or other inflammatory stimuli.

For example, the LFA-1 antagonist can inhibit IL-2 release from peripheral blood mononuclear cells (PBMCs) in primary culture stimulated with SEB with an IC50 or EC50 of 10 mM or less. In other embodiments, the LFA-1 antagonist inhibits IL-2 release from peripheral blood mononuclear cells (PBMCs) in primary culture stimulated with SEB with an IC50 or EC50 of 1 mM or less. In yet another embodiment, the LFA-1 antagonist inhibits IL-2 release from peripheral blood mononuclear cells (PBMCs) in primary culture stimulated with SEB with an IC50 or EC50 of 100 μM or less. The LFA-1 antagonist may inhibit IL-2 release from peripheral blood mononuclear cells (PBMCs) in primary culture stimulated with SEB with an IC50 or EC50 of 10 μM or less. In some embodiments, the LFA-1 antagonist inhibits IL-2 release from peripheral blood mononuclear cells (PBMCs) in primary culture stimulated with SEB with an IC50 or EC50 of approximately 1 μM, 100 nM, 10 nM, 1 nM or less.

In some embodiments, the LFA-1 antagonist simultaneously inhibits the release of two or more inflammatory cytokines with an IC50 or EC50 of approximately 1 μM or less when PBMC's are stimulated with SEB. In another embodiment, the LFA-1 antagonist simultaneously inhibits the release of two or more cytokines with an IC50 or EC50 of approximately 100 nM or less when PBMCs are stimulated with SEB. For example, the LFA-1 antagonist may simultaneously inhibit the release of IL-2 and IL-4 with an IC50 or EC50 of approximately 500 nM or less when PBMCs are stimulated with SEB. This can be important, without being bound by theory, because IL-2 and IL-4 release play important roles in Th1 and Th2 lymphocyte mediated inflammatory diseases. In another embodiment, the LFA-1 antagonist can simultaneously inhibit the release of IL-1(α), IL-1(β), IL-2, IL-4, IL-5, IL-10, IL-13, Interferon γ, MIP 1(α), MCP-1, TNF(α) and GM-CSF with an IC50 or EC50 of approximately 1 μM or less when PBMCs are stimulated with SEB.

The LFA-1 antagonist is delivered such that a local therapeutically effective concentration is achieved. For example, the therapeutically effective concentration may be achieved with a local tissue concentration of LFA-1 of greater than about 1 nM. In another embodiment, the local therapeutically effective concentration may be achieved with a local tissue concentration of LFA-1 of greater than about 10 nM. In some other embodiments, the local therapeutically effective concentration may be achieved with a local tissue concentration of LFA-1 of greater than about 100 nM. In yet another embodiment, the local therapeutically effective concentration may be achieved with a local tissue concentration of LFA-1 of greater than about 1 μM. In other embodiments, the local therapeutically effective concentration may be achieved with a local tissue concentration of LFA-1 of greater than about 10 μM. In another embodiment, the local therapeutically effective concentration of is achieved while maintaining a low systemic level. For example, in some embodiments, a local therapeutically effective concentration of about 1 nM, about 10 nM, about 100 nM, about 1 μM, or about 10 μM is achieved while maintaining a systemic drug concentration of less than 1 μM. In other embodiments, a local therapeutically effective concentration of about 1 nM, about 10 nM, about 100 μM, about 1 μM, or about 10 μM is achieved while maintaining a systemic drug concentration of less than 100 nM. In yet other embodiments, a therapeutically effective concentration of about 1 nM, about 10 nM, about 100 nM, about 1 μM, or about 10 μM is achieved while maintaining a systemic drug concentration of less than 10 nM. The invention provides other embodiments wherein a therapeutically effective concentration of about 1 nM, about 10 nM, about 100 nM, about 1 μM, or about 10 μM is achieved with a systemic drug concentration of less than 1 nM. The systemic drug concentration may be measured by blood plasma concentration using any of a variety of methods known in the art and as disclosed above.

In another aspect of the invention, the local tissue concentration of LFA-1 antagonist is maintained at therapeutically effective levels for an extended period of time. In some embodiments, it may be desired that local tissue concentrations of an LFA-1 antagonist is maintained at therapeutically effective levels for a certain amount of time or between doses. By selecting for LFA-1 antagonists that can maintain local therapeutically effective levels for extended periods, the subject may achieve a therapeutic effect without administration of multiple doses per day. In some embodiments, LFA-1 antagonists of the present invention, when delivered to a gastrointestinal tissue and absorbed into the gastrointestinal tissue, are maintained at concentrations above at least about 10 nM, about 50 nM, about 100 nM, about 150 nM, about 200 nM, about 300 nM, about 400 nM, about 500 nM, about 600 nm, about 700 nM, about 800 nM, about 900 nM, about 1 μM, about 2 μM, about 3 μM, about 4 μM, about 5 μM, about 8 μM, about 10 μM, about 12 μM, about 15 μM, about 18 μM, about 20 μM, about 30 μM, about 40 μM, or about 50 μM for as long as approximately 1, 2, 3, 5, 8, 10, 12, 14, 15, 16 18, 20, 22, or 24 hours post dose or administration. For example, the LFA-1 antagonist upon delivery can have a local tissue concentration of greater than 1 μM for at least 2 hours when administered to a subject. The concentrations and time may vary depending on the gastrointestinal organ or tissue. The local therapeutic level may be measured by any of a variety of methods known in the art, such as radiolabelled analysis.

In some embodiments, the LFA-1 antagonist has a local tissue concentration of greater than about 1 μM for at least about 2 hours, about 4 hours, about 6 hours, about 8 hours, about 10 hours, about 12 hours, about 14 hours, about 16 hours, about 18 hours, about 20 hours, about 22 hours, or about 24 hours following administration to a subject.

In other embodiments, the LFA-1 antagonist has a local tissue concentration of greater than about 100 nM for at least about 2 hours, about 4 hours, about 6 hours, about 8 hours, about 10 hours, about 12 hours, about 14 hours, about 16 hours, about 18 hours, about 20 hours, about 22 hours, or about 24 hours following administration to a subject.

In yet other embodiments, the LFA-1 antagonist has a local tissue concentration of greater than about 10 nM for at least about 2 hours, about 4 hours, about 6 hours, about 8 hours, about 10 hours, about 12 hours, about 14 hours, about 16 hours, about 18 hours, about 20 hours, about 22 hours, or about 24 hours following administration to a subject. In other embodiments, the LFA-1 antagonist is maintained at a local tissue concentration level greater than about 10 nM for up to about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours.

The invention also provides embodiments wherein the LFA-1 antagonist has a local tissue concentration of greater than about 1 nM for at least about 2 hours, about 4 hours, about 6 hours, about 8 hours, about 10 hours, about 12 hours, about 14 hours, about 16 hours, about 18 hours, about 20 hours, about 22 hours, or about 24 hours following administration to a subject.

### LFA-1 Antagonists

Specific LFA-1 antagonist compounds have been previously described in the art and may be used in the present invention. For example, LFA-1 antagonists have been described in U.S. Pat. No. 7,314,938, US Patent Application Publication No. 2006/0281739, U.S. application Ser. No. 12/288,330, and co-pending US Applications WSGR Docket Numbers 32411-712.201, 32411-708.201, and 32411-709.201; the contents of each of which are expressly incorporated herein by reference. The compounds can be synthesized as described in these references.

Exemplary molecules that may be used as LFA-1 antagonists are compounds of Formula (I) or (II):

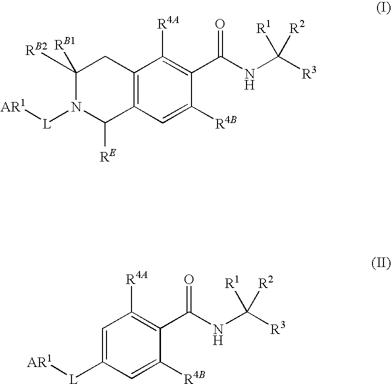

Wherein R¹ and R² are each independently hydrogen, an amino acid side chain, —(CH₂)ₘOH, —(CH₂)ₘaryl, —(CH₂)ₘheteroaryl, wherein m is 0-6, —CH(R^{1A})(OR^{1B}), —CH(R^{1A})(NHR^{1B}), U-T-Q, or an aliphatic, alicyclic, heteroaliphatic or heteroalicyclic moiety optionally substituted with U-T-Q,

wherein U is absent, —O—, —S(O)₀₋₂—, —SO₂N(R^{1A}), —N(R^{1A})—, —N(R^{1A})C(═O)—, —N(R^{1A})C(═O)—O—, —N(R^{1A})C(═O)—N(R^{1B})—, —N(R^{1A})—SO₂—, —C(═O)—, —C(═O)—O—, —O—C(═O)—, aryl, heteroaryl, alkylaryl, alkylheteroaryl, —C(═O)—N(R^{1A})—, —OC(═O)N(R^{1A})—, —C(═N—R^{1E})—, —C(═N—R^{1E})—O—, —C(═N—R^{1E})—N(R^{1A})—, —O—C(═N—R^{1E})—N(R^{1A})—, —N(R^{1A})C(═N—R^{1E})—, —N(R^{1A})C(═N—R^{1E})—O—, —N(R^{1A})C(═N—R^{1E})—N(R^{1B})—, —P(═O)(OR^{1A})—O—, or —P(═O)(R^{1A})—O—;
T is absent, an aliphatic, heteroaliphatic, aryl, heteroaryl, alkylaryl or alkylheteroaryl moiety; and
Q is hydrogen, halogen, cyano, isocyanate, —OR^{1B}; —SR^{1B}; —N(R^{1B})₂, —NHC(═O)OR¹⁸, —NHC(═O)N(R^{1B})₂, —NHC(═O)R^{1B}, —NHSO₂R^{1B}, —NHSO₂N(R^{1B})₂, —NHSO₂NHC(═O)OR^{1B}, —NHC(═O)NHSO₂R^{1B}, —C(═O)NHC(═O)OR^{1B}, —C(═O)NHC(═O)R^{1B}, —C(═O)NHC(═O)N(R^{1B})₂, —C(═O)NHSO₂R^{1B}, —C(═O)NHSO₂N(R^{1B})₂, —C(═S)N(R^{1B})₂, —SO₂R^{1B}, —SO₂OR^{1B}, —SO₂N(R^{1B})₂, —SO₂—NHC(═O)OR^{1B}, —OC(═O)—N(R^{1B})₂, —OC(═O)R^{1B}, —OC(═O)NHC(═O)R^{1B}, —OC(═O)NHSO₂R^{1B}, —OSO₂R^{1B}, or an aliphatic heteroaliphatic, aryl or heteroaryl moiety, or wherein R¹ and R² taken together are an alicyclic or heterocyclic moiety, or together are

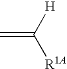

wherein each occurrence of R^{1A} and R^{1B} is independently hydrogen, an aliphatic, alicyclic, heteroaliphatic, heterocyclic, aryl, heteroaryl, alkylaryl or alkylheteroaryl moiety, —C(═O)R^{1C}, or —C(═O)NR^{1C}R^{1D}; wherein each occurrence of R^{1C} and R^{1D} is independently hydrogen, hydroxyl, or an aliphatic, heteroaliphatic, aryl, heteroaryl, alkylaryl or alkylheteroaryl moiety; and R^{1E} is hydrogen, an aliphatic, alicyclic, heteroaliphatic, heterocyclic, aryl, heteroaryl, alkylaryl or alkylheteroaryl moiety, —CN, —OR^{1C}, —NR^{1C}R^{1D} or —SO₂R^{1C};
R³ is —C(═O)OR^{3A}, —C(═O)H, —CH₂OR^{3A}, —CH₂C(═O)-alkyl, —C(═O)NH(R^{3A}), —CH₂X⁰; wherein each occurrence of R^{3A} is independently hydrogen, a protecting group, an aliphatic, alicyclic, heteroaliphatic, heteroalicyclic, aryl, heteroaryl, alkylaryl, alkylheteroaryl, heteroalkylaryl heteroalkylheteroaryl moiety, or pharmaceutically acceptable salt or ester, or R^{3A}, taken together with R¹ and R², forms a heterocyclic moiety; wherein X⁰ is a halogen selected from F, Br or I;
wherein R^{4A} and R^{4B} are independently a halogen selected from F, Cl, Br or I; and R^{B1}, R^{B2} and R^{E} are independently hydrogen or substituted or unsubstituted lower alkyl;
AR¹ is a monocyclic or polycyclic aryl, heteroaryl, alkylaryl, alkylheteroaryl, alicyclic or heterocyclic moiety; and,
L is absent or is V-W-X-Y-Z, wherein each occurrence of V, W, X, Y and Z is independently absent, C═O, NR^{L1}, —O—, —C(R^{L1})═, ═C(R^{L1})—, —C(R^{L1})(R^{L2}), C(═N—OR^{L1}), C(═NR^{L1}), —N═, S(O)₀₋₂; a substituted or unsubstituted C₁₋₆ alkenylidene or C₂₋₆ alkenylidine chain wherein up to two non-adjacent methylene units are independently optionally replaced by —C(═O)—, —CO₂—, —C(═O)C(═O)—, —C(C═O)NR^{L3}—, —OC(═O)—, —OC(═O)NR^{L3}—, —NR^{L3}NR^{L4}—, —NR^{L3}NR^{L4}C(═O)—, —NR^{L3}C(═O)—, NR^{L3}CO₂—, NR^{L3}C(═O)NR^{L4}, —S(═O)—, —SO₂—, —NR^{L3}SO₂—, —SO₂NR^{L3}, —NR^{L3}SO₂NR^{L4}, —O—, —S—, or —NR^{L3}—; wherein each occurrence of R^{L3} and R^{L4} is independently hydrogen, alkyl, heteroalkyl, aryl, heteroaryl or acyl; or an aliphatic, alicyclic, heteroaliphatic, heteroalicyclic, aryl, heteroaryl, alkylaryl or alkylheteroaryl moiety; and each occurrence of R^{L1} and R^{L2} is independently hydrogen, hydroxyl, protected hydroxyl, amino, protected amino, thio, protected thio, halogen, cyano, isocyanate, carboxy, carboxyalkyl, formyl, formyloxy, azido, nitro, ureido, thioureido, thiocyanato, alkoxy, aryloxy, mercapto, sulfonamido, benzamido, tosyl, or an aliphatic, alicyclic, heteroaliphatic, heteroalicyclic, aryl, heteroaryl, alkylaryl or alkylheteroaryl moiety, or wherein one or more occurrences of R^{L1} and R^{L2}, taken together, or taken together with one of V, W, X, Y or Z form an alicyclic or heterocyclic moiety or form an aryl or heteroaryl moiety, and/or its pharmaceutically acceptable salts or esters.

Compounds of the present invention include the following:

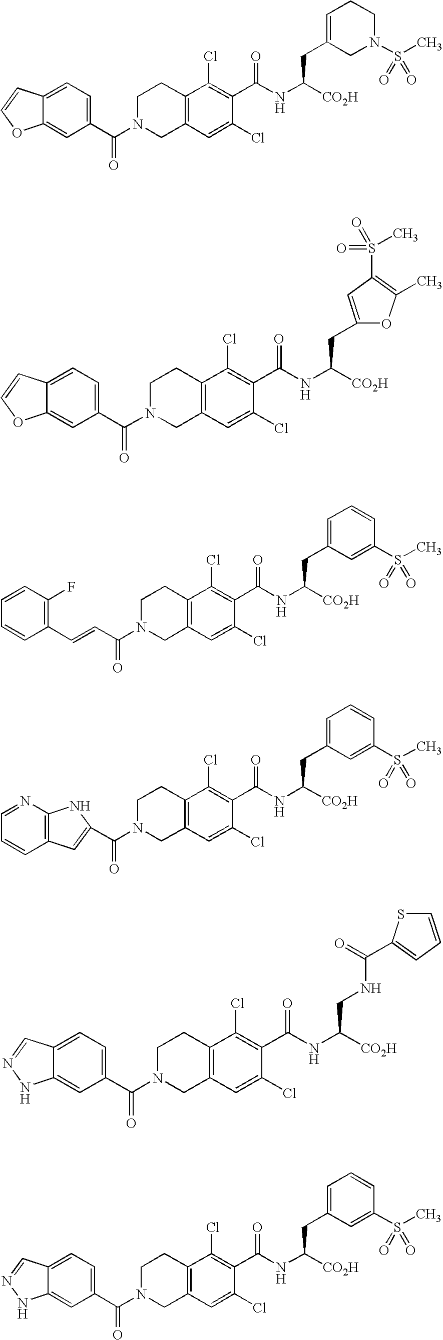

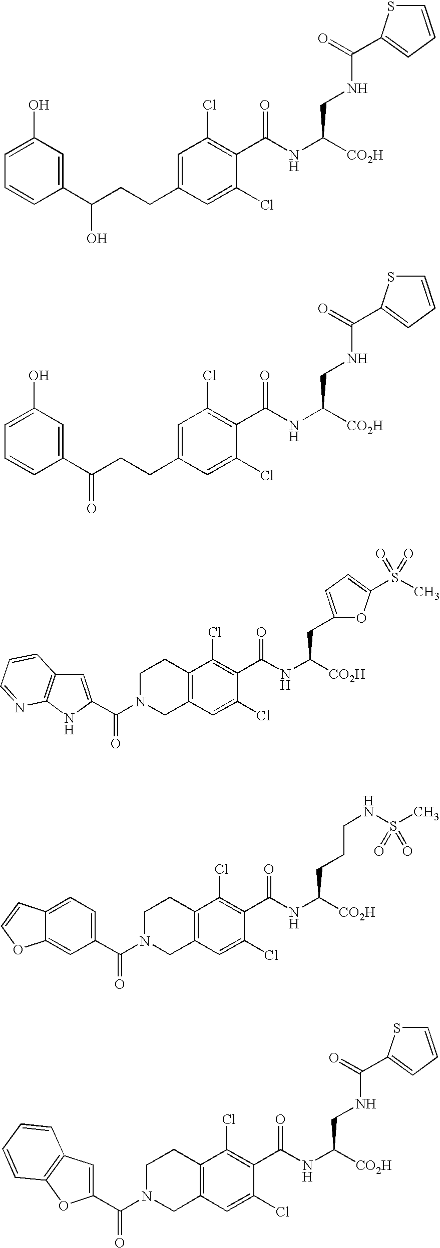

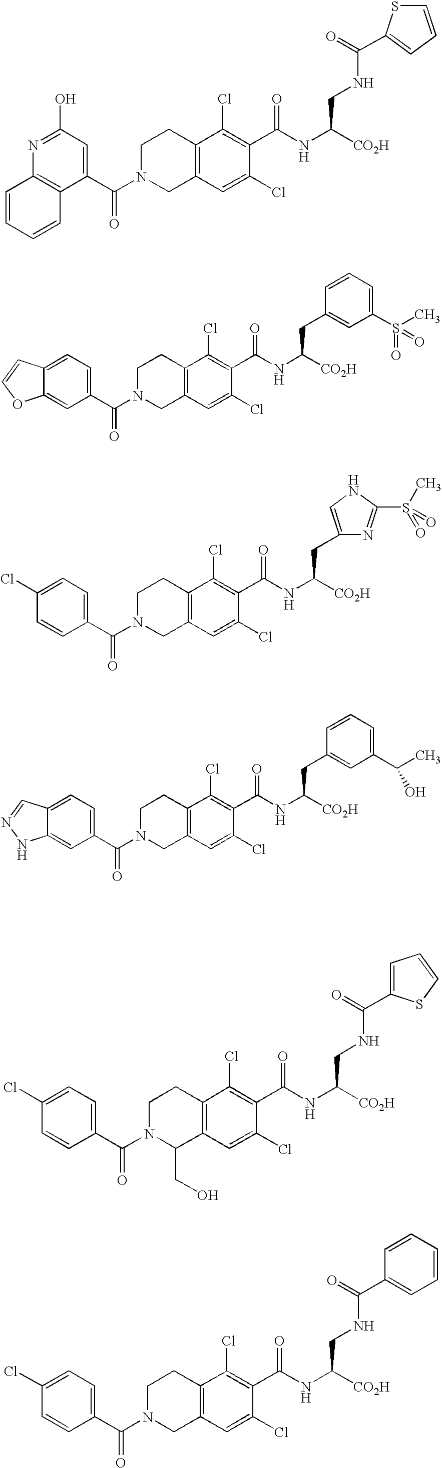

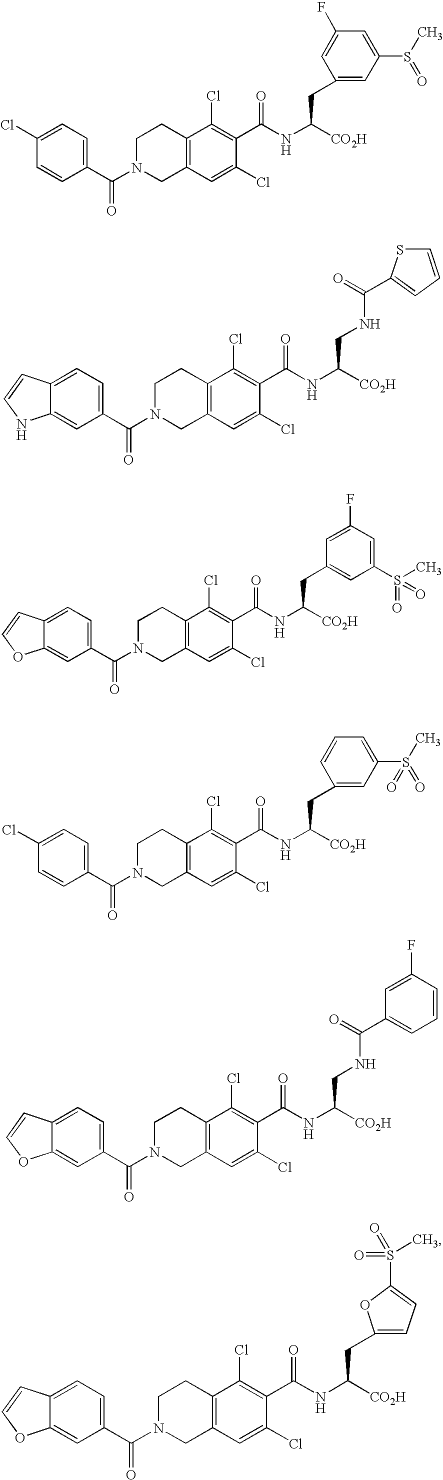

and their pharmaceutically acceptable salts and esters.

It is envisioned additionally, that the LFA-1 antagonist may be used in amorphous form or the LFA-1 antagonist may be any of the crystalline forms described in co-pending application docket number 32411-712.101. In some embodiments of the invention, the compound of Formula (I) is Form A of Compound 12, which comprises an X-ray powder diffraction pattern having characteristic peaks at a reflection angle 2θ of about 18.2, 21.4, and 22.7 degrees; Form B of Compound 12, which comprises an X-ray powder diffraction pattern having characteristic peaks at a reflection angle 2θ of about 12.1, 17.1, and 18.5 degrees; Form C of Compound 12, which comprises an X-ray powder diffraction pattern having characteristic peaks at a reflection angle 2θ of about 4.8, 17.8, and 21.5 degrees; Form D of Compound 12, which comprises an X-ray powder diffraction pattern having characteristic peaks at a reflection angle 2θ of about 17.6, 21.7, and 24.8 degrees; Form E of Compound 12, which comprises an X-ray powder diffraction pattern having characteristic peaks at a reflection angle 2θ of about 5.12, 8.26, and 17.8 degrees; an amorphous form of Compound 12, which comprises greater than 90% purity; or any combination thereof.

In some embodiments, the LFA-1 antagonist of Formula I or Formula II is a salt. Representative alkali or alkaline earth metal salts include but are not limited to sodium, lithium, potassium, calcium, and magnesium. Further pharmaceutically acceptable salts include, when appropriate, nontoxic ammonium, quaternary ammonium, and amine cations formed by direct reaction with the drug carboxylic acid or by using counterions such as halide, hydroxide, carboxylate, sulfate, phosphate, nitrate, sulfonate and aryl sulfonate. In one embodiment, the LFA-1 antagonist is used in the methods of the invention, as the sodium salt of the free carboxylic acid.

Antibodies specific for binding to LFA-1 may be used in the present invention. Blocking of the CAMs, such as for example ICAM-1, or the leukointegrins, such as LFA-1, by antibodies directed against either or both of these molecules can inhibit an inflammatory response. Previous studies have investigated the effects of anti-CD11a MAbs on many T-cell-dependent immune functions in vitro and a number of immune responses in vivo. In vitro, anti-CD11a MAbs inhibit T-cell activation (See Kuypers T. W., Roos D. 1989 “Leukocyte membrane adhesion proteins LFA-1, CR3 and p150,95: a review of functional and regulatory aspects” Res. Immunol., 140:461-465; Fischer A, Durandy A, Sterkers G, Griscelli C. 1986 “Role of the LFA-1 molecule in cellular interactions required for antibody production in humans” J. Immunol., 136, 3198; target cell lysis by cytotoxic T-lymphocytes (Krensky et al., supra), formation of immune conjugates (Sanders V M, Snyder J M, Uhr J W, Vitetta E S., “Characterization of the physical interaction between antigen-specific B and T cells”. J. Immunol., 137:2395 (1986); Mentzer S J, Gromkowski S H, Krensky A M, Burakoff S J, Martz E. 1985 “LFA-1 membrane molecule in the regulation of homotypic adhesions of human B lymphocytesn” J. Immunol., 135:9), and the adhesion of T-cells to vascular endothelium (Lo S K, Van Seventer G A, Levin S M, Wright S D., Two leukocyte receptors (CD11a/CD18 and CD11b/CD18) mediate transient adhesion to endothelium by binding to different ligands., J. Immunol., 143:3325 (1989)). Two anti-CD11a MAbs, HI 111, and G43-25B are available from Pharmingen/BD Biosciences and may be used. The anti-murine monoclonal antibody M17 has been studied for treatment of LFA-1 mediated disorders in mouse models of human disease and therapy (U.S. Pat. No. 5,622,700) and may be used. Additionally, a study including F8.8, CBR LFA 1/9, BL5, May.035, TS1/11, TS1/12, TS1/22, TS2/14, 25-3-1, MHM2 and efalizumab evaluated the range of binding sites on LFA-1 these antibodies occupied in blocking ICAM binding and leukocyte function. See Lu, C; et al. 2004, “The Binding Sites for Competitive Antagonistic, Allosteric Antagonistic, and Agonistic Antibodies to the I Domain of Integrin LFA-1” J. Immun. 173: 3972-3978 and references therein. For example, it has been shown that greater than 90% occupancy of LFA-1 with efalizumab led to a greater than 50% clinical improvement in PASI score in a clinical trial demonstrating the efficacy of efalizumab (see D. L. Mortenson et al. J Clin Pharmacol 2005; 45:286-298. “Pharmacokinetics and Pharmacodynamics of Multiple Weekly Subcutaneous Efalizumab Doses in Patients With Plaque Psoriasis”)

Peptides have also been investigated for use in reducing the interaction of LFA-1 with ICAM-1 and may be used in the present invention. Polypeptides that do not contain an Fc region of an IgG are described in U.S. Pat. No. 5,747,035, and can be used to treat LFA-1 mediated disorders, in particular diabetic retinopathy. Use of dual peptides, the first a modulator of ICAM-1 and the second a blocking peptide with a sequence obtained from LFA-1 is described in U.S. Pat. No. 5,843,885 to reduce the interactions between LFA-1 and ICAM-1 can also be used. Cyclic peptides have been described in U.S. Pat. No. 6,630,447 as inhibitors of the LFA-1: ICAM-1 interaction and are also provided in the present invention.

Small molecule antagonists can also be used in the present invention, for example, statins, which bind to the CD11a domain of LFA-1, can be used. See Kallen, J., Welzenbach, K., Ramage, P. Geyl, D. Kriwacki, R., Legge, G., Cottens, S., Weitz-Schrnidt, G., and Hommel, U. 1999. “Structural basis for LFA-1 inhibition upon lovastatin binding to the CD11a I-domain”, J. Mol. Biol., 292: 1-9; and Weitz-Schmidt, G., Welzenbach, K., Brinkmann, V., Kamata, T., Kallen, J., Bruns, C., Cottens, S., Takada, Y., and Hommel, U. 2001. Statins, without being bound by theory, selectively inhibit leukocyte function antigen-1 by binding to a novel regulatory integrin site (Nature Med., 7: 687-692; and Frenette, P. S. 2001. “Locking a leukocyte integrin with statins”, N. Engl. J. Med., 345: 1419-1421). Molecules derived from the mevinolin/compactin motif also show activity against LFA-1, and can be used in the present invention. See Welzenbach, K. et al., 2002. “Small molecule inhibitors induce conformational changes in the I domain and the I-like domain of Lymphocyte Function-Associated Antigen-1”, J. Biol. Chem., 277: 10590-10598, and U.S. Pat. No. 6,630,492.

Additionally, other known LFA-1 antagonists recognized in the art may be used in the present invention. For example, a family of hydantoin-based inhibitors can be used as LFA-1 antagonists. See Kelly, T. A. et al., 1999. “Cutting edge: a small molecule antagonist of LFA-1-mediated cell adhesion”, J. Immunol., 163: 5173-5177. These compounds are believed to be allosteric inhibitors of LFA-1. As another example, a family of novel p-arylthio cinnamides can act as antagonists of LFA-1. See Liu, G. et al., 2000 “Discovery of novel p-arylthio cinnamides as antagonists of leukocyte function-associated antigen-1/intracellular adhesion molecule-1 interaction. 1. Identification of an additional binding pocket based on an anilino diaryl sulfide lead.” J. Med. Chem. 43, 4015-4030.

Other families of small molecule inhibitors are disclosed, for example as described in Gadek, T. R et al., 2002. “Generation of an LFA-1 antagonist by the transfer of the ICAM-1 immunoregulatory epitope to a small molecule” Science, 295: 1086-1089 and online supplementary material.) and in U.S. Pat. No. 6,872,735, U.S. Pat. No. 6,667,318, U.S. Pat. No. 6,803,384, U.S. Pat. No. 6,515,124, U.S. Pat. No. 6,331,640, as well as in U.S. Patent Applications 20020119994, 20040058968, 20050080119, and in PCT applications WO99/49856, WO00/21920, WO01/58853, WO02/59114, WO05/044817, and others. The contents of all the cited references are incorporated in their entirety by reference.

### Formulations for LFA-1 Antagonists

The formulations of LFA-1 antagonists of the present invention provides for gastrointestinal targeted delivery. Compounds of the invention exhibit low, moderate or high systemic oral bioavailability and are capable of achieving drug levels in GI tissue of 100 nM or greater. Accordingly, in various embodiments, compounds of the invention are administered via oral delivery. Pharmaceutical compositions of the present invention include, but are not limited to, solids, solutions, emulsions, and liposome-containing formulations. These compositions may be generated from a variety of components that include, but are not limited to, preformed liquids, self-emulsifying solids and self-emulsifying semisolids.

The LFA-1 antagonist may be formulated as the free carboxylic acid, a salt of the free carboxylic acid, or an ester of the free carboxylic acid. Representative alkali or alkaline earth metal salts include but are not limited to sodium, lithium, potassium, calcium, and magnesium. Further pharmaceutically acceptable salts include, when appropriate, nontoxic ammonium, quaternary ammonium, and amine cations formed by direct reaction with the drug carboxylic acid. The LFA-1 antagonist can be converted to simple alkyl esters, including but not limited to methyl, ethyl, propyl, butyl and pentyl ester, by known methods of preparation to provide a prodrug form of the LFA-1 antagonist. An ester prodrug of the LFA-1 antagonist can be absorbed readily across GI epithelium and converted to drug once past the epithelial surface or circulated to liver and converted to drug in the liver. After such conversion, the active drug is directed back into the GI via bile. The LFA-1 antagonist may be formulated in solution or as a suspension of the solid drug.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product. Formulations may also be for aerosolized delivery.

The compositions of the present invention may be formulated into any of many possible dosage forms such as, but not limited to, tablets, capsules, liquid syrups, soft gels, suppositories, and enemas. Formulations may include powders or granules, microparticles, nanoparticles, suspensions or solutions in aqueous, non-aqueous or mixed media, capsules (including but not limited to hard capsules, and soft elastic capsules), sachets or tablets. Aqueous suspensions may further contain substances that increase the viscosity of the suspension including, for example, sodium carboxymethylcellulose, sorbitol and/or dextran. The suspension may also contain stabilizers.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, anti-viral agents, or may contain additional materials useful in formulating various dosage forms of the compositions of the present invention, such as dyes, flavoring agents, preservatives, antioxidants, opacifiers, thickening agents, diluents, emulsifiers, dispersing aids or binders, lubricants, surface active or dispersing agents, humectants, stabilizers, anti-caking agents, preservatives, sweetening agents, colorants, desiccants, plasticizers, dyes, binders, fillers, disintegrants, anti-microbial agents, coating agents, and the like. Any such optional ingredient should be compatible with the compound of the invention to insure the stability of the formulation. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like.

The composition may contain additives as needed, including for example lactose, glucose, fructose, galactose, trehalose, sucrose, maltose, raffinose, maltitol, melezitose, stachyose, lactitol, palatinite, starch, xylitol, mannitol, myoinositol, and the like, and hydrates thereof, and amino acids, for example alanine, glycine and betaine, and peptides and proteins, for example albumen.

Examples of binders used in the present invention can include, but not limited to, corn starch, potato starch, other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch (e.g., STARCH 1500™ and STARCH 1500 LM™, sold by Colorcon, Ltd.), hydroxypropyl methyl cellulose, microcrystalline cellulose (e.g. AVICEL™, such as, AVICEL-PH-101™, -103™ and -105™, sold by FMC Corporation, Marcus Hook, Pa., USA), or mixtures thereof.

Fillers that may be used include, but not be limited to: talc, calcium carbonate (e.g., granules or powder), dibasic calcium phosphate, tribasic calcium phosphate, calcium sulfate (e.g., granules or powder), microcrystalline cellulose, powdered cellulose, dextrates, kaolin, mannitol, silicic acid, sorbitol, starch, pre-gelatinized starch, or mixtures thereof.

Disintegrants such as, but not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums, or mixtures thereof may also be used.

Examples of lubricants that maybe used include, but are not limited to: calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil and soybean oil), zinc stearate, ethyl oleate, ethyl laurate, agar, syloid silica gel (AEROSIL 200, W.R. Grace Co., Baltimore, Md. USA), a coagulated aerosol of synthetic silica (Deaussa Co., Plano, Tex. USA), a pyrogenic silicon dioxide (CAB-O-SIL, Cabot Co., Boston, Mass. USA), and mixtures thereof.

Anti-caking agents, such as: calcium silicate, magnesium silicate, silicon dioxide, colloidal silicon dioxide, talc, or mixtures thereof, can also be used. Optionally, the formulations may include antimicrobial agents such as: benzalkonium chloride, benzethonium chloride, benzoic acid, benzyl alcohol, butyl paraben, cetylpyridinium chloride, cresol, chlorobutanol, dehydroacetic acid, ethylparaben, methylparaben, phenol, phenylethyl alcohol, phenoxyethanol, phenylmercuric acetate, phenylmercuric nitrate, potassium sorbate, propylparaben, sodium benzoate, sodium dehydroacetate, sodium propionate, sorbic acid, thimersol, thymo, or mixtures thereof.

The excipients may be, but not limited to, phosphate buffered saline solutions, propylene glycol diesters of medium chain fatty acids available under the tradename Miglyol 840 (from Huls America, Inc. Piscataway, N.J.) triglyceride esters of medium chain fatty acids available under the tradename Miglyol 812 (from Huls); perfluorodimethylcyclobutane available under the tradename Vertrel 245 (from E. I. DuPont de Nemours and Co. Inc. Wilmington, Del.); perfluorocyclobutane available under the tradename octafluorocyclobutane (from PCR Gainesville, Fla.); polyethylene glycol available under the tradename EG 400 (from BASF Parsippany, N.J.); menthol (from Pluess-Stauffer International Stanford, Conn.); propylene glycol monolaurate available under the tradename lauroglycol (from Gattefosse Elmsford, N.Y.), diethylene glycol monoethylether available under the tradename Transcutol (from Gattefosse); polyglycolized glyceride of medium chain fatty adds available under the tradename Labrafac Hydro WL 1219 (from Gattefosse); alcohols, such as ethanol, methanol and isopropanol; eucalyptus oil available (from Pluses-Stauffer International): and mixtures thereof. Compositions may also include amino acid derivatives.

Formulations may also comprise other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate. Self-emulsifying drug delivery systems (SEDDS), which are isotropic mixtures of oils, surfactants, solvents and co-solvents/surfactants, (i.e. SEDDS, or self microemulsifying drug delivery system (SMEDDS) or similar emulsifying agents, (for example, Gelucir™) can be used in formulations of the LFA-1 antagonist. Emulsion formulations are also appropriate, including oil-in-water and water-in-oil emulsions.

Other excipients may include water, buffered aqueous solutions, surfactants, volatile liquids, starches, polyols, gelatin, lactose, amylose, magnesium stearate, talc, silicic acid, granulating agents, hydroxymethylcellulose, cyclodextrins, polyvinylpyrrolidone, microcrystalline cellulose, diluents, lubricants, acids, bases, salts, emulsions, such as oil/water emulsions, oils such as mineral oil and vegetable oil, wetting agents, chelating agents, antioxidants, sterile solutions, complexing agents, disintegrating agents and the like. Buffered solutions will typically be at physiological pH and typically be buffered to the pH of the target tissue.

Surfactants can be used to form pharmaceutical compositions and dosage forms of the invention. They may include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, preferred ionic surfactants include, by way of example: lecithins, lysolecithin, phospholipids, lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl camitines, myristoyl camitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with glycerides, vegetable oils, hydrogenated vegetable oils, fatty acids, or sterols; polyoxyethylene sterols, derivatives, or analogues thereof; polyoxyethylated vitamins or derivatives thereof; polyoxyethylene-polyoxypropylene block copolymers; or mixtures thereof; polyethylene glycol sorbitan fatty acid esters or hydrophilic transesterification products of a polyol with triglycerides, vegetable oils, or hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-1100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with glycerides, vegetable oils, hydrogenated vegetable oils, fatty acids and sterols; oil-soluble vitamins/vitamin derivatives; or mixtures thereof. Within this group, lipophilic surfactants can be glycerol fatty acid esters, propylene glycol fatty acid esters, or mixtures thereof, or are hydrophobic transesterification products of a polyol and vegetable oils, hydrogenated vegetable oils, or triglycerides.

Surfactants may be used in any formulation of the invention where its use is not otherwise contradicted. In some embodiments of the invention, surfactants may not be used, or limited classes or amounts of surfactants are used.

Compositions may be administered via oral delivery. Oral formulations can comprise liquid formulations which are encapsulated or not. A liquid formulation may be an aqueous solution of the LFA-1 antagonist, and may contain buffering agents and may or may not have preservatives included. Orally administered formulations such as tablets may optionally be coated or scored and may be formulated so as to provide sustained, delayed or controlled release of the active ingredient therein. Examples of solid formulations may be as described in U.S. Pat. No. 5,424,289. Oral formulations can also have increased bioavailability, such as described in U.S. Pat. No. 7,097,851, location and time dependent in delivery, such as described in U.S. Pat. No. 5,840,332, or delivered to specific regions of the gastrointestinal system, for example, as described in U.S. Pat. No. 5,849,327, where coating of an enteric material that remains intact until the dosage form reaches the lower gastrointestinal tract.

Formulations may use enteric coatings which are available for tablets and capsules. Enteric coatings can remain intact in the stomach but rapidly dissolve when they arrive at the small intestine, thereafter releasing the drug at sites downstream in the intestine (e.g., the ileum and colon), thus delivering a LFA-1 antagonist to the mucosa thereof. Enteric coatings are well known in the art and are discussed at, for example, Remington's Pharmaceutical Sciences, Mack Publishing Co., Easton, Pa.; and Polymers for Controlled Drug Delivery, Chapter 3, CRC Press, 1991. Some non-limiting examples of enteric coatings include cellulose acetate phthalate, polyvinyl acetate phthalate, methacrylic acid-methacrylic acid ester copolymers, carboxymethyl ethylcellulose, and hydroxypropyl methylcellulose acetate succinates. Alternatively, a controlled release oral delivery vessel designed to release the formulations comprising a LFA-1 antagonist after a predetermined period of time, and thus after the vessel has passed into the ileum or colon, can also be used to deliver the formulation of the present invention. Such vessels include, but are not limited to, the CHRONSET™ delivery device (ALZA Corporation, Palo Alto, Calif.) and the Pulsincap™ delivery device (R.P. Scherer Co.). Other coating agents may include, but not be limited to: sodium carboxymethyl cellulose, cellulose acetate phthalate, ethylcellulose, gelatin, pharmaceutical glaze, hydroxypropyl cellulose, hydroxypropyl methylcellulose, hydroxypropyl methyl cellulose phthalate, methylcellulose, polyethylene glycol, polyvinyl acetate phthalate, shellac, sucrose, titanium dioxide, carnauba wax, microcrystalline wax, or mixtures thereof.

Controlled release oral formulations of the LFA-1 antagonist can also be formed wherein the LFA-1 antagonist is incorporated within a biocompatible and/or biodegradable matrix. The matrix can be hydrophilic or hydrophobic. Three main mechanisms exist by which an active ingredient can be released from a hydrophilic matrix: dissolution, erosion and diffusion. An active ingredient will be released by the dissolution mechanism when it is homogeneously dispersed in a matrix network of a soluble polymer. The network will gradually dissolve in the gastrointestinal tract, thereby gradually releasing its load. The matrix polymer can also gradually be eroded from the matrix surface, likewise releasing the active ingredient in time. When an active ingredient is processed in a matrix made up of an insoluble polymer, it will be released by diffusion: the gastro-intestinal fluids penetrate the insoluble, sponge-like matrix and diffuse back out loaded with drug.

The formulations of the present invention can contain the LFA-1 antagonist as either a carboxylic acid or as a salt. The formulations can include a polymer such as polylactic-glycoloic acid (PLGA), poly-(I)-lactic-glycolic-tartaric acid (P(I)LGT) (WO 01/12233), polyglycolic acid (U.S. Pat. No. 3,773,919), polylactic acid (U.S. Pat. No. 4,767,628), poly(M-caprolactone) and poly(alkylene oxide) (U.S. 2003/0068384) to create a sustained release formulation, which may be liquid, gel, or a solid. Such formulations can be used to manufacture implants that release a LFA-1 antagonist over a period of a few days, a few weeks or several months depending on the polymer, the particle size of the polymer, and the size of the implant (see, e.g., U.S. Pat. No. 6,620,422). Other sustained release formulations and polymers for use in are described in EP 0 467 389 A2, WO 93/24150, U.S. Pat. No. 5,612,052, WO 97/40085, WO 03/075887, WO 01/01964A2, U.S. Pat. No. 5,922,356, WO 94/155587, WO 02/074247A2, WO 98/25642, U.S. Pat. No. 5,968,895, U.S. Pat. No. 6,180,608, U.S. 20030171296, U.S. 20020176841, U.S. Pat. No. 5,672,659, U.S. Pat. No. 5,893,985, U.S. Pat. No. 5,134,122, U.S. Pat. No. 5,192,741, U.S. Pat. No. 5,192,741, U.S. Pat. No. 4,668,506, U.S. Pat. No. 4,713,244, U.S. Pat. No. 5,445,832 U.S. Pat. No. 4,931,279, U.S. Pat. No. 5,980,945, WO 02/058672, WO 9726015, WO 97/04744, and US20020019446. In sustained release formulations forming implants, microparticles of LFA-1 antagonist are combined with microparticles of polymer. One or more sustained release implants can be placed in the large intestine, the small intestine or both. U.S. Pat. No. 6,011,011 and WO 94/06452 describe a sustained release formulation providing either polyethylene glycols (i.e. PEG 300 and PEG 400) or triacetin.

Another formulation which may both enhance bioavailability and provide controlled release of the LFA-1 antagonist within the GI tract, is a variant of that described in WO 03/053401. Such a controlled release formulation includes a permeation enhancer, the LFA-1 antagonist, and a carrier that exhibits in-site gelling properties, such as a nonionic surfactant. The formulation is delivered within the GI tract as a liquid having at least some affinity for the surface of the GI mucosal membrane. Once released, the liquid formulation can spread across one or more areas on the surface of the GI mucosal membrane, where the carrier of the formulation then transitions into a bioadhesive gel in-situ. As a bioadhesive gel, the formulation of the present invention not only adheres to the mucosal membrane of the GI tract, but also reduces or minimizes dilution of both the permeation enhancer and the LFA-1 antagonist included in the formulation by lumenal fluids and secretions. Bioavailability of the LFA-1 antagonist may be increased by presenting the LFA-1 antagonist, together with a suitable permeation enhancer, at the surface of the mucosal membrane of the GI tract at concentrations sufficient to increase absorption of the LFA-1 antagonist through the GI mucosal membrane over a period of time.

Permeation enhancers suitable for use in a controlled formulation of this type include, but are not limited to, ethylene-diamine tetra-acetic acid (EDTA), bile salt permeation enhancers, such as sodium deoxycholate, sodium taurocholate, sodium deoxycholate, sodium taurodiliydrofusidate, sodium dodecylsulfate, sodium glycocholate, taurocholate, glycocholate, taurocheno-deoxycholate, taurodeoxycholate, deoxycholate, glycodeoxycholate, and ursodeoxycholate, fatty acid permeation enhancers, such as sodium caprate, sodium laurate, sodium caprylate, capric acid, lauric acid, and caprylic acid, acyl carnitines, such as palmitoyl carnitine, stearoyl camitine, myristoyl carnitine, and lauroyl carnitine, and salicylates, such as sodium salicylate, 5-methoxy salicylate, and methyl salicylate. Permeation enhancers may act to open the tight junctions formed between epithelial cells of the GI mucosal membrane, and thereby allow diffusion of the LFA-1 antagonist into the intestinal mucosa (i.e., pericellular absorption. Though the amount of permeation enhancer included in the formulation of the present invention may range from about 10 wt % to about 40 wt %, the nature and precise amount of permeation enhancer included in the formulation of the present invention will vary depending on, for example, the LFA-1 antagonist to be delivered, the nature of the permeation enhancer itself, and the dose of formulation to be administered. The amount of permeation enhancer included in the formulation should be sufficient to maintain an effective concentration of permeation enhancer (i.e., a concentration above the critical concentration for the permeation enhancer used) at or near the surface of the GI mucosal membrane over a period of time sufficient to increase the bioavailability of the LFA-1 antagonist. Where possible, the permeation enhancer can be chosen such that the permeation enhancer not only facilitates absorption of the LFA-1 antagonist, but also resists dilution by lumenal fluids or secretions. Permeation enhancers may also be used in formulations of the invention which are not controlled release formulations.

The carrier of a controlled release formulation containing a permeation enhancer, the LFA-1 antagonist, and the carrier exhibiting in-site gelling properties will permit a transition from a relatively non-adhesive, low viscosity liquid to a relatively viscous, bioadhesive gel after the formulation has been delivered within the GI tract of a subject. The carrier is chosen such that the transition from a relatively non-adhesive, low viscosity liquid to a relatively viscous, bioadhesive gel occurs after the formulation has been released within the GI tract and had some opportunity to arrive at the surface of the GI mucosal membrane. Hence, the carrier of the formulation of the present invention enables the in-situ transition of the formulation from a liquid to a bioadhesive gel. Due to its high viscosity and bioadhesive properties, the gel formed by the formulation of the present invention holds the permeation enhancer and the LFA-1 antagonist together at the surface of the GI mucosal membrane and protects both such components from dilution and enzymatic degradation over a period of time. Suitable carriers include non-ionic surfactants that transition from a relatively non-adhesive, low viscosity liquid to a relatively viscous, bioadhesive liquid crystal state as they absorb water. Specific examples of non-ionic surfactants that may be used as the carrier in the formulation of the present invention include, but are not limited to, Cremophor (e.g., Cremophor EL and Cremophor RH), Incordas 30, polyoxyethylene 5 castor oil, polyethylene 9 castor oil, polyethylene 15 castor oil, d-a-tocopheryl polyethylene glycol succinate (TPGS), monoglycerides, such as myverol, aliphatic alcohol based nonionic surfactants, such as oleth-3, oleth-5, polyoxyl 10 oleyl ether, oleth-20, steareth-2, steareth-10, steareth-20, ceteareth-20, polyoxyl 20 cetostearyl ether, PPG-5 ceteth-20, and PEG-6 capryl/capric triglyceride, Pluronic@ and tetronic block copolymer non-ionic surfactants, such as Pluronic® L10, L31, L35, L42, L43, L44, L62, L61, L63, L72, L81, L101, L121, and L122, polyoxylene sorbitan fatty acid esters, such as Tween 20, Tween 40, Tween 60, Tween 65, Tween 80, Tween 81, and Tween 85, and ethoxylated glycerides, such as PEG 20 almond glycerides, PEG-60 almond glycerides, PEG-20 corn glycerides, and PEG-60 corn ARC 2921 PCT 11 glycerides. The carrier may be present in about 35 wt % to about 88 wt % of the formulation.

As water is added to the controlled release formulation having a non-ionic surfactant as the carrier, the initial viscosity of the formulation will increase. However, as water content increases, the increase in viscosity of nonionic surfactants tends to be non-linear. Often, as the water content of a nonionic surfactant exceeds a certain threshold, the viscosity of the nonionic surfactant increases rapidly as the nonionic surfactant transitions to its gelling state. If a relatively quick conversion is desired, a formulation including a nonionic surfactant may be provided more water, thereby placing the formulation closer to the water content threshold at which the formulation will rapidly convert to a bioadhesive gel. In contrast, if a relatively slow conversion is desired, the formulation may include less water or no water, thereby placing the formulation farther from the gelling threshold.

Additionally, the controlled release formulation containing a permeation enhancer, the LFA-1 antagonist, and the carrier exhibiting in-site gelling properties may also include a viscosity reducing agent that reduces the initial viscosity of the formulation. Reducing the initial viscosity of the formulation may further facilitate spreading of the formulation of the present invention across one or more areas of the GI mucosal membrane after the formulation is delivered within the GI tract but before the formulation transitions into a bioadhesive gel.

Exemplary viscosity reducing agents that may be used include, but are not limited to, polyoxyethylene 5 castor oil, polyoxyethylene 9 castor oil, labratil, labrasol, capmul GMO (glyceryl mono oleate), capmul MCM (medium chain mono- and diglyceride), capmul MCM C8 (glyceryl mono caprylate), capmul MCM C10 (glyceryl mono caprate), capmul GMS-50 (glyceryl mono stearate), caplex 100 (propylene glycol didecanoate), caplex 200 (propylene glycol dicaprylate/dicaprate), caplex 800 (propylene glycol di 2-ethyl hexanoate), captex 300 (glyceryl tricapryl/caprate), captex 1000 (glyceryl tricaprate), captex 822 (glyceryl triandecanoate), captex 350 (glyceryl tricaprylate/caprate/laurate), caplex 810 (glyceryl tricaprylate/caprate/linoleate), capmul PG8 (propylene mono caprylate), propylene glycol, and propylene glycol laurate (PGL). Where a viscosity reducing agent is included, the viscosity reducing agent may be present in amounts up to about 10 wt % of the formulation.

Further, the dosage form of the controlled release formulation containing a permeation enhancer, the LFA-1 antagonist, and the carrier exhibiting in-site gelling properties may include a hard or soft gelatin capsule. In some embodiments, the dosage form is designed, such as by use of enteric coatings, to delay release of the formulation until the dosage form has passed through the stomach and at least entered the small intestine.

Additional controlled release formulations are described in WO 02/38129, EP 326 151, U.S. Pat. No. 5,236,704, WO 02/30398, WO 98/13029; U.S. 20030064105, U.S. 20030138488A1, U.S. 20030216307A1, U.S. Pat. No. 6,667,060, WO 01/49249, WO 01/49311, WO 01/49249, WO 01/49311, and U.S. Pat. No. 5,877,224. An example of a solid controlled release formulation may include hydrophilic polymers such as starch, cellulosic polymers, polyacrylic acids, or polymethacrylic acids to entrap the LFA-1 antagonist; cyclodextrins such as alpha, beta, or gamma cyclodextrins and further including substituted cyclodextrins such as sulfobutyl cyclodextrins or hydroxypropyl beta cyclodextrin, which complex with the LFA-1 antagonist and act to provide a more regulated release of the LFA-1 antagonist from the solid controlled release formulation.

The oral administration formulations may utilize gastroretentive formulations to enhance absorption from the gastrointestinal (GI) tract. A formulation which is retained in the stomach for several hours may release compounds of the invention to provide a sustained release in the upper gastrointestinal region that may be desirable in some embodiments of the invention. Disclosure of such gastro-retentive formulations are found in Klausner, E. A.; Lavy, E.; Barta, M.; Cserepes, E.; Friedman, M.; Hoffman, A. 2003 “Novel gastroretentive dosage forms: evaluation of gastroretentivity and its effect on levodopa in humans.” Pharm. Res. 20, 1466-73, Hoffman, A.; Stepensky, D.; Lavy, E.; Eyal, S. Klausner, E.; Friedman, M. 2004 “Pharmacokinetic and pharmacodynamic aspects of gastroretentive dosage forms” Int. J. Pharm. 11, 141-53, Streubel, A.; Siepmann, J.; Bodmeier, R.; 2006 “Gastroretentive drug delivery systems” Expert Opin. Drug Deliver. 3, 217-3, and Chavanpatil, M.D.; Jain, P.; Chaudhari, S.; Shear, R.; Vavia, P. R. “Novel sustained release, swellable and bioadhesive gastroretentive drug delivery system for olfoxacin” Int. J. Pharm. 2006 epub March 24. Expandable, floating and bioadhesive techniques may be utilized to maximize the extent and/or duration of absorption of the compounds of the invention. Materials such as cross povidone, pysllium husk, chitosan, cellulosic polymers, amongst other materials, can be selected and combined to vary the buoyancy lag time, duration of buoyancy, dimensional stability, drug content and drug release profile. Variation of the physical characteristics of the formulation can also used to vary these parameters of drug delivery. For example, a biodegradable membrane may be included as part of a gastroretentive formulation, which membrane is buoyant in the stomach and is exposed to the gastric environment only over a predetermined time period. This membrane may be formed of materials that only release LFA-1 antagonist after this initial exposure period, thus, in combination with an immediately releasing portion of the gastroretentive formulation, providing LFA-1 antagonist over a much extended period of time relative to a formulation comprising only immediate release compositions.

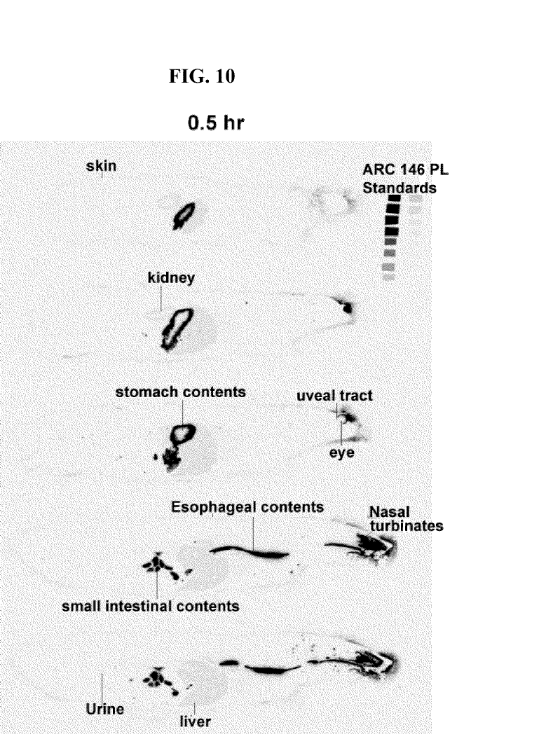

Formulations for intranasal administration may utilize an aerosol suspension of respirable particles comprised of LFA-1 antagonists, which the individual inhales. The LFA-1 antagonist of the invention contact the lacrimal tissues via nasolacrimal ducts, contact the nasal passageways, or contact the oral cavity, and subsequently be delivered to the gastrointestinal mucosa in a pharmaceutically effective amount (see FIG. 10 and Example 10). The respirable particles may be solid or liquid, with suitably sized particles, as is known in the art to be effective for absorption. Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Compositions in pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, orally or nasally, from devices that deliver the formulation in an appropriate manner. The formulation of LFA-1 antagonists may be combined with a propellant (e.g. in a metered dose inhaler), and used with any of a variety of nebulizers, or by dry powder inhalers. The aerosol formulations can allow for efficacious delivery of LFA-1 antagonists to mucosal surfaces of the gastrointestinal system.

Alternatively, the LFA-1 antagonist is formulated for administration as a suppository. Coating as discussed above can be used to extend the release time of drug from the suppository. The formulations for suppositories may also comprise sustained release or slow release matrices, microparticles or nanoparticles.

The concentration of drug may be adjusted, the pH of the solution buffered and the isotonicity adjusted to be compatible the route of administration, as is well known in the art.

In some embodiments, the formulation has a pH between about 4.5 to about 7.5, between about 5.0 to about 7.5, between about 5.5 to about 7.5, between about 6.0 to about 7.5, or about 6.5 to about 7.5.

The LFA-1 antagonists of the present invention may be milled to provide more suitable properties for formulation. Milling may provide smaller particle size with greater surface area exposure, which can provide faster solubilization in-vivo or during formulation. Alternatively, milling to a smaller particle size may provide the capacity of the LFA-1 antagonist to pass through biological barriers, such as the skin or gut wall, directly, without initial solubilization, permitting the use of the LFA-1 antagonist as a solid in the formulation, which may provide additional benefits of temperature stability, shelf life, ease of transport, and ease of use by the subject. Milled solid particles of the LFA-1 antagonist may also provide greater bioavailability, and more desirable or controllable pharmacokinetics in the formulations. The size of the milled particle can affect the rate of distribution of the LFA-1 antagonist upon administration or rate of release of the LFA-1 antagonist from a sustained or slow release formulation. Further, milling of the particles of the LFA-1 antagonist may be performed to create either a narrower or more symmetrical particle size distribution within a particular formulation or lot of material which may be subjected to formulation. The size of the particles of the LFA-1 antagonist may be selected as is well known in the art, to obtain the desired physical characteristics for ease of formulation or the ability to be distributed from the formulation in a controlled fashion over a preselected period under conditions of use. The size of the particles can be represented as the D50, which represents the median or 50′ percentile of the diameter of a particle within the lot of material under discussion. Another measure of the size of the particles in a lot of material is the D90, which is the 90^{th} percentile of the particle size diameter in the particle size distribution.

In the formulations of the invention, the diameter of the particles of the LFA-1 antagonist is in the range from about 5 nm to about 100 μm, from about 50 nm to about 100 μm, from about 100 nm to about 100 μm, from about 250 nm to about 100 μm, from about 500 nm to about 100 μm, from about 750 nm to about 100 μm, from about 1 μm to about 100 μm, or from about 10 μm to about 100 μm; from about 5 nm to about 50 μm, from about 50 nm to about 50 μm, from about 100 μm to about 50 μm, from about 250 nm to about 50 μm, from about 500 nm to about 50 μm, from about 750 nm to about 50 μm, from about 1 μm to about 50 μm, or from about 10 μm to about 50 μm; from about 5 nm to about 10 μm, from about 50 nm to about 10 μm, from about 100 nm to about 10 μm, from about 250 nm to about 10 μm, from about 500 nm to about 10 μm, from about 750 nm to about 10 μm, or from about 1 μm to about 10 μm; from about 5 nm to about 1 μm, from about 50 nm to about 1 μm, from about 100 nm to about 1 μm, from about 250 nm to about 1 μm, from about 500 nm to about 1 μm, or from about 750 nm to about 1 μm; from about 5 nm to about 1 μm, from about 50 nm to about 1 μm, from about 100 nm to about 1 μm, from about 250 nm to about 1 μm, from about 500 nm to about 1 μm, or from about 750 nm to about 1 μm; from about 5 nm to about 1 μm, from about 50 nm to about 1 μm, from about 100 nm to about 1 μm, from about 250 nm to about 1 μm, from about 500 nm to about 1 μm, or from about 750 nm to about 1 μm; from about 5 nm to about 900 nm, from about 50 nm to about 900 nm, from about 100 nm to about 900 nm, from about 250 nm to about 900 nm, from about 500 nm to about 900 nm, or from about 750 nm to about 900 nm; from about 5 nm to about 900 nm, from about 50 nm to about 900 nm, from about 10 nm to about 900 nm, from about 250 nm to about 900 nm, from about 500 nm to about 900 nm, or from about 750 nm to about 900 nm; from about 5 nm to about 900 nm, from about 50 nm to about 900 nm, from about 100 nm to about 900 nm, from about 250 nm to about 900 nm, from about 500 nm to about 900 nm, or from about 750 nm to about 900 nm; from about 5 nm to about 750 nm, from about 50 nm to about 750 nm, from about 100 mm to about 750 nm, from about 250 nm to about 750 nm, from about 500 nm to about 750 nm, from about 750 nm to about 750 nm, or from about 1 μm to about 750 nm; from about 5 nm to about 750 nm, from about 50 nm to about 750 nm, from about 100 nm to about 750 nm, from about 250 nm to about 750 nm, or from about 500 nm to about 750 nm; from about 5 nm to about 750 nm, from about 50 nm to about 750 nm, from about 100 nm to about 750 nm, from about 250 nm to about 750 nm, or from about 500 nm to about 750 nm; from about 5 nm to about 500 nm, from about 50 nm to about 500 nm, from about 100 nm to about 500 nm, or from about 250 nm to about 500 nm; from about 5 nm to about 250 m, from about 50 nm to about 250 nm, or from about 100 nm to about 250 nm; from about 5 nm to about 500 nm, from about 10 nm to about 500 nm, from about 20 nm to about 500 nm, from about 30 nm to about 500 nm, from about 40 nm to about 500 nm, from about 50 nm to about 500 nm, from about 60 nm to about 500 nm, from about 70 nm to about 500 nm, from about 80 nm to about 500 nm, from about 90 nm to about 500 nm, from about 100 nm to about 500 nm, from about 200 nm to about 500 nm, or from about 300 nm to about 500 nm.

In the formulations of the invention, the D50 of the diameter of the particles of the LFA-1 antagonist are about 100 μm, about 90 μm, about 80 μm, about 70 μm, about 60 μm, about 50 μm, about 45 μm, about 40 μm, about 35 μm, about 30 μm, about 25 μm, about 20 μm, about 19 μm, about 18 μm, about 17 μm, about 16 μm, about 15 μm, about 14 μm, about 13 μm, about 12 μm, about 11 μm, about 10 μm, about 9 μm, about 8 μm, about 7 μm, about 6 μm, about 5 μm, about 4 μm, about 3 μm, about 2 μm, about 1 μm, about 950 nm, about 900 nm, about 850 nm, about 800 nm, about 750 nm, about 700 nm, about 650 nm, about 600 nm, about 550 nm, about 500 nm, about 450 nm, about 350 nm, about 300 nm, about 250 nm, about 200 nm, about 150 nm, about 100 nm, about 95 nm, about 90 nm, about 85 nm, about 80 nm, about 75 nm, about 70 nm, about 65 nm, about 60 nm, about 55 nm, about 50 nm, about 45 nm, about 40 nm, about 35 nm, about 30 nm, about 25 nm, about 20 nm, about 19 nm, about 18 nm, about 17 nm, about 16 nm, about 15 nm, about 14 nm, about 13 nm, about 12 nm, about 11 nm, about 10 nm, about 9 nm, about 8 nm, about 7 nm, about 6 nm, or about 5 nm.

In the formulations of the invention, the D50 of the diameter of the particles of the LFA-1 antagonist are less than about 100 μm, about 90 μm, about 80 μm, about 70 μm, about 60 μm, about 50 μm, about 45 μm, about 40 μm, about 35 μm, about 30 μm, about 25 μm, about 20 μm, about 19 μm, about 18 μm, about 17 μm, about 16 μm, about 15 μm, about 14 μm, about 13 μm, about 12 μm, about 10 μm, about 10 μm, about 9 μm, about 8 μm, about 7 μn, about 6 μm, about 5 μm, about 4 μm, about 3 μm, about 2 μm, about 1 μm, about 950 nm, about 900 nm, about 850 nm, about 800 nm, about 750 nm, about 700 nm, about 650 nm, about 600 nm, about 550 nm, about 500 nm, about 450 nm, about 350 nm, about 300 nm, about 250 nm, about 200 nm, about 150 nm, about 100 nm, about 95 nm, about 90 nm, about 85 nm, about 80 nm, about 75 nm about 70 nm, about 65 nm, about 60 nm, about 55 nm, about 50 nm, about 45 nm, about 40 nm, about 35 nm, about 30 nm, about 25 nm, about 20 nm, about 19 nm, about 18 nm, about 17 nm, about 16 nm, about 15 nm, about 14 nm, about 13 nm, about 12 nm, about 11 nm, about 10 nm, about 9 nm, about 8 nm, about 7 nm, about 6 nm, or about 5 nm.

In the formulations of the invention, the D50 of the diameter of the particles of the LFA-1 antagonist is no more than about 100 μm, about 90 μm, about 80 μm, about 70 μm, about 60 μm, about 50 μm, about 45 μm, about 40 μm, about 35 μm, about 30 μm, about 25 μm, about 19 μm, about 18 μm, about 17 μm, about 16 μm, about 20 μm, about 15 μm, about 14 μm, about 13 μm, about 12 μm, about 11 μm, about 10 μm, about 9 μm, about 8 μm, about 7 μm, about 6 μm, about 5 μm, about 4 μm, about 3 μm, about 2 μm, about 1 μm, about 950 nm, about 900 nm, about 850 nm, about 800 nm, about 750 nm, about 700 nm, about 650 nm, about 600 nm, about 550 nm, about 500 nm, about 450 nm, about 350 nm, about 300 nm, about 250 nm, about 200 nm, about 150 nm, about 100 nm, about 95 nm, about 90 nm, about 85 nm, about 80 nm, about 75 nm, about 70 nm, about 65 nm, about 60 nm, about 55 nm, about 50 nm, about 45 nm, about 40 nm, about 35 nm, about 30 nm, about 25 nm, about 20 nm, about 19 nm, about 18 nm, about 17 nm, about 16 nm, about 15 nm, about 14 nm, about 13 nm, about 12 nm, about 11 nm, about 10 nm, about 9 nm, about 8 nm, about 7 nm, about 6 nm, or about 5 nm.

In the formulations of the invention, the D90 of the diameter of the particles of the LFA-1 antagonist is about 100 μm, about 90 μm, about 80 μm, about 70 μm, about 60 μm, about 50 μm, about 45 μm, about 40 μm, about 35 μm, about 30 μm, about 25 μm, about 20 μm, about 19 μm, about 18 μm, about 17 μm, about 16 μm, about 15 μm, about 14 μm, about 13 μm, about 12 μn, about 11 μm, about 10 μm, about 9 μm, about 8 μm, about 7 μm, about 6 μm, about 5 μm, about 4 μm, about 3 μm, about 2 μm, about 1 μm, about 950 nm, about 900 nm, about 850 nm, about 800 nm, about 750 nm, about 700 nm, about 650 nm, about 600 nm, about 550 nm, about 500 nm, about 450 nm, about 350 nm, about 300 nm, about 250 nm, about 200 nm, about 150 nm, about 100 nm, about 95 nm, about 90 nm, about 85 nm, about 80 nm, about 75 nm, about 70 nm, about 65 nm, about 60 nm, about 55 nm, about 50 nm, about 45 nm, about 40 nm, about 35 nm, about 30 nm, about 25 nm, about 20 nm, about 19 nm, about 18 nm, about 17 nm, about 16 nm, about 15 nm, about 14 nm, about 13 nm, about 12 nm, about 11 nm, about 10 nm, about 9 nm, about 8 nm, about 7 nm, about 6 nm, or about 5 nm.

In the formulations of the invention, the D90 of the diameter of the particles of the LFA-1 antagonist is less than about 100 μm, about 90 μm, about 80 μm, about 70 μm, about 60 μm, about 50 μm, about 45 μm, about 40 μm, about 35 μm, about 30 μm, about 25 μm, about 20 μm, about 19 μm, about 18 μm, about 17 μm, about 16 μm, about 15 μm, about 14 μm, about 13 μm, about 12 μm, about 11 μm, about 10 μm, about 9 μm, about 8 μm, about 7 μm, about 6 μm, about 5 μm, about 4 μm, about 3 μm, about 2 μm, about 1 μm, about 950 nm, about 900 nm, about 850 nm, about 800 nm, about 750 nm, about 700 nm, about 650 nm, about 600 nm, about 550 nm, about 500 nm, about 450 nm, about 350 nm, about 300 nm, about 250 nm, about 200 nm, about 150 nm, about 100 nm, about 95 nm, about 90 nm, about 85 nm, about 80 nm, about 75 nm, about 70 nm, about 65 nm, about 60 nm, about 55 nm, about 50 nm, about 45 nm, about 40 nm, about 35 nm, about 30 nm, about 25 nm, about 20 nm, about 19 nm, about 18 nm, about 17 nm, about 16 nm, about 15 nm, about 14 nm, about 13 nm, about 12 nm, about 11 nm, about 10 nm, about 9 nm, about 8 nm, about 7 nm, about 6 nm, or about 5 nm.

In the formulations of the invention, the D90 of the diameter of the particles of the of the LFA-1 antagonist is no more than about 100 μm, about 90 μm, about 80 μm, about 70 μm, about 60 μm, about 50 μm, about 45 μm, about 40 μm, about 35 μm, about 30 μm, about 25 μm, about 20 μm, about 19 μm, about 18 μm, about 17 μm, about 16 μm, about 15 μm, about 14 μm, about 13 μm, about 12 μm, about 11 μm, about 10 μm, about 9 μm, about 8 μm, about 7 μm, about 6 μm, about 5 μm, about 4 μm, about 3 μm, about 2 μm, about 1 μm, about 950 nm, about 900 nm, about 850 nm, about 800 nm, about 750 nm, about 700 nm, about 650 nm, about 600 nm, about 550 nm, about 500 nm, about 450 nm, about 350 nm, about 300 nm, about 250 nm, about 200 nm, about 150 nm, about 100 nm, about 95 nm, about 90 nm, about 85 nm, about 80 nm, about 75 nm, about 70 nm, about 65 nm, about 60 nm, about 55 nm, about 50 nm, about 45 nm, about 40 nm, about 35 nm, about 30 nm, about 25 nm, about 20 nm, about 19 nm, about 18 nm, about 17 nm, about 16 nm, about 15 nm, about 14 nm, about 13 nm, about 12 nm, about 11 nm, about 10 nm, about 9 nm, about 8 nm, about 7 nm, about 6 nm, or about 5 nm.

For delivery to the gastrointestinal mucosa of a human, the formulations comprising the LFA-1 antagonist may range in concentration from about 5.0 to 10.0 W/W % of the LFA-1 antagonist. In some other embodiments, the formulation comprises about 1.0 W/W %, about 2.0 W/W %, about 3.0 W/W %, about 4.0 W/W %, about 5.0 W/W %, about 6.0 W/W %, about 7.0 W/W %, about 8.0 W/W %, about 9.0 W/W % or about 10.0 W/W % of the LFA-1 antagonist. In other embodiments, the formulation comprises about 10.0 W/W %, about 12.0 W/W %, about 14.0 W/W %, about 15.0 W/W %, about 16.0 W/W %, about 17.0 W/W %, about 18.0 W/W %, about 20.0 W/W %, about 21.0 W/W %, about 22.0 W/W %, about 23.0 W/W %, about 24.0 W/W %, or about 25.0 W/W % of the LFA-1 antagonist, In yet other embodiments of the invention, the formulation comprises about 25.0 W/W %, about 26 W/W %, about 27 W/W %, about 28 W/W %, about 29 W/W % about 30 W/W %, about 32 W/W %, about 34 W/W %, about 36 W/W % about 38 W/W %, about 40 W/W %, about 42 W/W %, about 44 W/W %, about 46 W/W %, about 48 W/W %, or about 50 W/W % of the LFA-1 antagonist. In further embodiments of the invention, the formulation comprises about 45 W/W %, about 50W/W %, about 55 W/W %, about 60W/W %, about 65 W/W %, about 70 W/W %, about 75 W/W %, about 80 W/W %, or about 85 W/W % of the LFA-1 antagonist.

The LFA-1 antagonist formulations can include other therapeutic agents, depending on the type of condition being treated. For example, when the condition being treated is an inflammatory bowel disease, the additional agent can be a corticosteroid, or other type of immunosuppressive agent. Further examples are described below.

### Administration of LFA-1 Antagonists to the Gastrointestinal System

Compositions of the present invention are therapeutically and/or prophylactically useful for treating diseases or conditions mediated by LFA-1 activity. Accordingly, a method of treating a disease or condition mediated by LFA-1 in subject, such as an animal, is provided in the present invention. The subject can refer to any animal, including but not limited to, human and non-human animals, such primates, ovines, bovines, ruminants, lagomorphs, porcines, caprines, equines, canines, felines, aves, murines, and others. For example, the present invention provides a method for treating an inflammatory disorder comprising administering to a human subject an effective amount of a compound of the invention.

Administration of the formulations described herein is useful for the treatment of leukocyte mediated inflammation. The formulations of the invention are potent inhibitors of LFA-1 and without being bound by theory, inhibit cytokines released by Th1 T-cells and Th2 T-cells. Leukocyte mediated inflammation plays a role in initiating and advancing inflammation in selected diseases, such as T cell inflammatory responses.

The formulation is administered in an amount effective to treat, prevent, or diagnose on one or more symptoms or manifestations of an immune of inflammatory related disease or disorder, examples further described below. An effective amount is an amount of the compound or formulation which upon administration is capable of reducing the activity of LFA-1; or the amount of compound required to prevent, inhibit or reduce the severity of any symptom associated with an LFA-1 mediated condition or disease upon administration. A therapeutically effective amount of a medicament of the present invention may be employed in pure form or, where such forms exist, in pharmaceutically acceptable salt, ester or prodrug form. The therapeutically effective amount of is typically meant a sufficient amount of the compound to obtain the intended therapeutic benefit, at a reasonable benefit/risk ratio applicable to any medical treatment. Local administration of the LFA-1 antagonist which is rapidly cleared from the systemic circulation may be particularly beneficial in this regard where the local to systemic exposure ratio may be 10 to 10,000 fold or more

It is understood, however, that the total daily usage of the medicaments and compositions of the present invention will be decided by the attending physician within the scope of sound medical judgment. The specific therapeutically effective dose level for any particular patient and medicament will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific medicament employed; the duration of the treatment; drugs used in combination or coincidental with the specific compound employed; and like factors well known in the medical arts. For example, it is well within the skill of the art to start doses at levels lower than required to achieve the desired therapeutic effect and to gradually increase the dosage until the desired effect is achieved.

The LFA-1 antagonist present may be an amount sufficient to exert a therapeutic effect to reduce symptoms of an immune or inflammatory related disorder or symptom by an average of at least about 5, 10, 15, 20, 25, 30, 40, 50, 60, 70, 80, or 90%. In some embodiments, the symptoms are reduced by greater than 90%, or substantially eliminated.

The formulations of the present invention can be administered by any suitable means, such as, but not limited to, topical, oral (including but not limited to sublingual, buccal, or inhalation), intranasally (including but not limited to aerosolly, nasal drops, or using a cannula), oral, or rectal via use of a suppository.

The formulations described herein can be administered locally to achieve therapeutically effective concentration locally and do not distribute systemically at pharmacologically effective concentrations, which may be by oral, depot, instillation, or pump administration. In some embodiments, the formulations with LFA-1 antagonists are administered orally or rectally with a slow release profile

If additional therapeutic agents are administered as separate compositions, they may be administered by the same route or by different routes. If additional therapeutic agents are administered in a single pharmaceutical composition it may be administered by any suitable route. In some embodiments, combinations of agents with one or more LFA-1 antagonists are administered as a single composition by oral administration. In some embodiments, combinations of therapeutic agents with one or more LFA-1 antagonists are administered as a single pharmaceutical composition by transdermal administration. In some embodiments, the combination of one or more additional therapeutic agents with one or more LFA-1 antagonists is administered as a single pharmaceutical composition by injection. In some embodiments, the combination of one or more therapeutic agents with one or more LFA-1 antagonists is administered as a single composition topically.

The compositions can also be administered with a pharmacokinetic profile that results in the delivery of an effective dose of the LFA-1 antagonist. The actual effective amounts of drug can vary according to the specific drug or combination thereof being utilized, the particular composition formulated, the mode of administration, and the age, weight, condition of the patient, and severity of the symptoms or condition being treated. Dosages for a particular patient can be determined by one of ordinary skill in the art using conventional considerations, (e.g. by means of an appropriate, conventional pharmacological protocol).

The benefits of administration of the formulations described herein include localized delivery of the therapeutic agent and minimal systemic side effects due to low systemic bioavailability. For example, an oral formulation delivers high concentrations of the LFA-1 antagonist once the dosage unit passes into lumen of the gastrointestinal system. The high local concentration will generate an osmotic gradient and drive drug locally into tissues of the GI exposed to the local gradient. Drug that is absorbed past the local tissue will be taken up into vasculature and be swept to the liver via the portal vein. The LFA-1 antagonist is cleared by the liver/bile and returned to the lower GI lumen, where it may be absorbed.

The therapeutic agents of the invention have a rapid systemic clearance such that any drug that gets absorbed systemically is quickly cleared. It is known that LFA-1 interacts with several ligands which could result in several unwanted side effects. In some embodiments, the local concentration of therapeutic agent is about 2×, 3×, 4×, 5×, 10×, 25×, 50×, or about 100× greater than the systemic concentration. In another embodiment of the present invention, local concentration of LFA-1 antagonist is about 1000× greater than the systemic concentration. In some embodiments, the local concentration is about 10,000× or more greater than the systemic concentration at the same time point. The concentration of therapeutic agent may be measured using any known method in the art. For example, radiolabelled therapeutic drug may be used and measurements taken from the local site of administration compared to systemic levels (e.g. plasma level concentrations).

The methods of treating a subject may involve the administration of one or more drugs for the treatment of one or more diseases. Combinations of agents can be used to treat one disease or multiple diseases or to modulate the side-effects of one or more agents in the combination. The other agents used in combination with LFA-1 antagonists include agents used to treat immune related disorders, and/or counteract certain effects, e.g. LFA-1 antagonists may be administered with drugs that cause dry eye as a side effect.

A therapeutic benefit may be achieved when there is eradication or amelioration of an inflammatory symptom. A therapeutic benefit may also be achieved when there is eradication or amelioration of the underlying disorder being treated. Alternatively, a therapeutic benefit may be achieved with the eradication or amelioration of one or more of the physiological symptoms associated with the underlying disorder. For example, a therapeutically effective amount comprising LFA-1 antagonists for treating inflammatory bowel disorder (IBD) may be defined as the dosage level for a subject such that the subject's symptoms of IBD are reduced, which refers to any degree of qualitative or quantitative reduction in detectable symptoms of IBD, including but not limited to, a detectable impact on the rate of recovery from disease (e.g. rate of weight gain), or the reduction of at least one of the following symptoms: abdominal pain, diarrhea, rectal bleeding, weight loss, fever, loss of appetite, dehydration, anemia, distention, fibrosis, inflamed intestines and malnutrition.

Treatment of a disorder or disease with compositions of the present invention may lead to an improvement observed in the subject, improvement perceived by the subject or physician, notwithstanding that the subject may still be afflicted with the underlying disorder. For example, systemic manifestations of the disease may still be present following local administration. For prophylactic benefit, the compositions may be administered to a subject at risk of developing a particular disease, or to a subject reporting one or more of the physiological symptoms of a disease, even though a diagnosis of this disease may not have been made. The compositions may be administered to a subject to prevent progression of physiological symptoms or of the underlying disorder.

The formulations or pharmaceutical compositions comprising the LFA-1 antagonist can be administered in a single dose. A single dose of a pharmaceutical composition comprising the LFA-1 antagonist may also be used when it is co-administered with another substance (e.g., an analgesic) for treatment of an acute condition. In some embodiments, a pharmaceutical composition comprising the LFA-1 antagonist is administered in multiple doses. Dosing may be about once, twice, three times, four times, five times, six times, seven times, eight times, nine times, ten times or more than ten times per day. Dosing may be about once a year, twice a year, every six months, every 4 months, every 3 months, every 60 days, once a month, once every two weeks, once a week, or once every other day.

In another embodiment the administration of the pharmaceutical composition comprising the LFA-1 antagonist continues for less than about 7 days. In yet another embodiment, the administration continues for more than about 6, 10, 14, 28 days, two months, six months, or one year. In some cases, dosing is maintained as long as necessary, e.g., dosing for chronic inflammation.

In another embodiment, a pharmaceutical composition comprising the LFA-1 antagonist is administered in combination with another therapeutic agent about once per day to about 10 times per day. In another embodiment the co-administration of the pharmaceutical composition comprising the LFA-1 antagonist with another therapeutic substance continues for less than about 7 days. In yet another embodiment the co-administration continues for more than about 6, 10, 14, 28 days, two months, six months, or one year. In some cases, co-administered dosing is maintained as long as necessary, e.g., dosing for chronic inflammation. In some embodiments, the co-administration is in the same composition.

In another embodiment, the co-administration is in separate pharmaceutical compositions. In some embodiments, the co-administration is concomitant. In some embodiments, the administration of the second therapeutic agents is before the administration of the pharmaceutical composition comprising the LFA-1 antagonist. In some embodiments, the administration of the second therapeutic agents is after the administration of the pharmaceutical composition comprising the LFA-1 antagonist. In one embodiment, the second therapeutic agent is an analgesic.

Administration of the compositions of the invention may continue as long as necessary. In some embodiments, a composition of the invention is administered for more than 1, 2, 3, 4, 5, 6, 7, 14, or 28 days. In some embodiments, a composition of the invention is administered for less than 28, 14, 7, 6, 5, 4, 3, 2, or 1 day. In some embodiments, a composition of the invention is administered chronically on an ongoing basis, e.g., for the treatment of chronic pain.

The amount of administration and the number of administrations of the active ingredient used in the present invention vary according to sex, age and body weight of patient, symptoms to be treated, desirable therapeutic effects, administration routes and period of treatment. Dosing for the methods of the invention may be found by routine experimentation. The daily dose can range from about 1×10⁻¹⁰ g to 5000 mg. Daily dose range may depend on the form of the formulations comprising the LFA-1 antagonists e.g., the esters or salts used, and/or route of administration, and/or solubility of the specific form (e.g. aqueous or solid). For example, for systemic administration, typical daily dose ranges are, e.g. about 1-5000 mg, or about 1-3000 mg, or about 1-2000 mg, or about 1-1000 mg, or about 1-500 mg, or about 1-100 mg, or about 10-5000 mg, or about 10-3000 mg, or about 10-2000 mg, or about 10-1000 mg, or about 10-500 mg, or about 10-200 mg, or about 10-100 mg, or about 20-2000 mg or about 20-1500 mg or about 20-1000 mg or about 20-500 mg, or about 20-100 mg, or about 50-5000 mg, or about 50-4000 mg, or about 50-3000 mg, or about 50-2000 mg, or about 50-1000 mg, or about 50-500 mg, or about 50-100 mg, about 100-5000 mg, or about 100-4000 mg, or about 100-3000 mg, or about 100-2000 mg, or about 100-1000 mg, or about 100-500 mg. In some embodiments, the daily dose of the formulation described herein is about 100, 200, 300, 400, 500, 600, 700, 800, 900, or 1000 mg. In some embodiments, the daily dose of the LFA-1 antagonist is 0.1 mg. In some embodiments, the daily dose of the LFA-1 antagonist is 1 mg. In some embodiments, the daily dose of the LFA-1 antagonist is 10 mg. In some embodiments, the daily dose of LFA-1 antagonist is 100 mg. In some embodiments, the daily dose of LFA-1 antagonist is 500 mg. In some embodiments, the daily dose the LFA-1 antagonist is 1000 mg.

In some embodiments, the LFA-1 antagonist is present in an amount sufficient to exert a therapeutic effect to reduce symptoms of a disorder mediated by LFA-1, by an average of at least about 5, 10, 15, 20, 25, 30, 40, 50, 60, 70, 80, 90, more than 90%, or substantially eliminate the symptoms of the disorder mediated by LFA-1.

In some embodiments, an effective amount of the LFA-1 antagonist is a daily dose of about 1×10⁻¹¹, 1×10⁻¹⁰, 1×10⁻⁹, 1×10⁻⁸, 1×10⁻⁷, 1×10⁻⁶, 1×10⁻⁵, 1×10⁻⁴, 1×10⁻³, 1×10⁻², 1×10⁻¹, 1, 1×10¹, 1×10² grams.

For topical delivery to the tissue surface the gastrointestinal organs, the typical daily dose ranges are, e.g. about 1×10⁻¹⁰ g to 5.0 g, or about 1×10⁻¹⁰ g to 2.5 g, or about 1×10⁻¹⁰ g to 1.00 g, or about 1×10⁻¹⁰ g to 0.5 g, or about 1×10⁻¹⁰ g to 0.25 g, or about 1×10⁻¹⁰ g to 0.1 g, or about 1×10⁻¹⁰ g to 0.05 g, or about 1×10⁻¹⁰ g to 0.025 g, or about 1×10⁻¹⁰ g to 1×10⁻² g, or about 1×10⁻¹ g to 5×10⁻³ g, or about 1×10⁻¹⁰ g to 2.5×10⁻³ g, or about 1×10⁻¹⁰ g to 1×10⁻³ g, or about 1×10⁻¹⁰ g to 5×10⁻⁴ g, or 1×10⁻¹⁰ g to 2.5×10⁻⁴ g, or about 1×10⁻¹⁰ g to 1×10⁻⁴ g, or about 1×10⁻¹⁰ g to 5×10⁻⁵ g, or 1×10⁻¹⁰ g to 2.5×10⁻⁵ g, or about 1×10⁻¹⁰ g to 1×10⁻⁵ g, or about 1×10⁻¹⁰ g to 5×10⁻⁶ g, or about 1×10⁻⁹ g to 1.00 g, or about 1×10⁻⁹ g to 0.5 g, or about 1×10⁻⁹ g to 0.25 g, or about 1×10⁻⁹ g to 0.1 g, or about 1×10⁻⁹ g to 0.05 g, or about 1×10⁻⁹ g to 0.025 g, or about 1×10⁻⁹ g to 1×10⁻² g, or about 1×10⁻⁹ g to 5×10⁻³ g, or about 1×10⁻⁹ g to 2.5×10⁻³ g, or about 1×10⁻⁹ g to 1×10⁻³ g, or about 1×10⁻⁹ g to 5×10⁻⁴ g, or about 1×10⁻⁸ g to 5.0 g, or about 1×10⁻⁸ g to 2.5 g, or about 1×10⁻⁸ g to 1 g, or about 1×10⁻⁸ g to 0.5 g, or about 1×10⁻⁸ g to 0.25 g, or about 1×10⁻³ g to 0.1 g, or about 1 to 5×10⁻² g, or about 1×10⁻⁸ to 5×10⁻² g, or about 1×10⁻⁸ g to 2.5×10⁻² g, or about 1×108 g to 1×10⁻² g, or about 1×10⁻⁸ g to 5×10⁻³ g, or about 1×10⁻⁸ to 2.5×10⁻³ g, or about 1×10⁻⁸ g to 1×10⁻³ g, or about 1×10⁻⁸ g to 5×10⁻⁴ g, or about 1×10⁻⁷ g to 5.0 g, or about 1×10⁻⁷ g to 2.5 g, or about 1×10⁻⁷ g to 1 g, or about 1×10⁻⁷ g to 0.5 g, or about 1×10⁻⁷ g to 0.25 g, or about 1×10⁻⁷ g to 0.1 g, or about 1×10⁻⁷ g to 5×10⁻² g, or about 1×10⁻⁷ to 5×10⁻² g, or about 1×10⁻⁷ g to 2.5×10⁻² g, or about 1×10⁻⁷ g to 1×10⁻² g, or about 1×10⁻⁷ g to 5×10⁻³ g, or about 1×10⁻⁷ to 2.5×10⁻³ g, or about 1×10⁻⁷ g to 1×10⁻³ g, or about 1×10⁻⁷ to 5×10⁻⁴ g, or about 1×10⁻⁶ g to 5.0 g, or about 1×10⁻⁶ g to 2.5 g, or about 1×10⁻⁶ g to 1 g, or about 1×10⁻⁶ g to 0.5 g, or about 1×10⁻⁶ g to 0.25 g, or about 1×10⁻⁶ g to 0.1 g, or about 1×10⁻⁶ g to 5×10⁻² g, or about 1×10⁻⁶ to 5×10⁻² g, or about 1×10⁻⁶ g to 2.5×10⁻² g, or about 1×10⁻⁶ g to 1×10⁻² g, or about 1×10⁻⁶ g to 5×10⁻³ g, or about 1×10⁻⁶ to 2.5×10⁻³ g, or about 1×10⁻⁶ g to 1×10⁻³ g, or about 1×10⁻⁶ g to 5×10⁻⁴ g, or about 1×10⁻⁵ g to 5 g, or about 1×10⁻⁵ g to 2.5 g, or about 1×10⁻⁵ g to 1 g, or about 1×10⁻⁵ g to 0.5 g, or about 1×10⁻⁵ g to 0.25 g, or about 1×10⁻⁵ g to 0.1 g, or about 1×10⁻⁵ g to 0.05 g, or about 1×10⁻⁵ g to 2.5×10⁻² g, or about 1×10⁻⁵ g to 1×10⁻² g, or about 1×10⁻⁵ g to 5×10⁻³ g, or about 1×10⁻⁵ g to 2.5×10⁻³ g, or about 1×10⁻⁵ g to 1×10⁻³ g, or about 1×10⁻⁵ g to 5×10⁻⁴ g.

In some embodiments, the daily dose of the LFA-1 antagonist is about 1×10⁻⁹, about 1×10⁻⁹, about 1×10⁻⁸, about 1×10⁻⁷, about 1×10⁻⁶, about 1×10⁻⁵, about 1×10⁻⁴, about 1×10⁻³ g, about 1×10⁻² g, about 1×10¹ g, or about 1 g. In some embodiments, the daily dose of the LFA-1 antagonist is about 1×10⁻¹⁰ g. In some embodiments, the daily dose of LFA-1 antagonist is about 1×10⁻⁹ g. In some embodiments, the daily dose of the LFA-1 antagonist is about 1×10⁻⁸ g. In some embodiments, the daily dose of the LFA-1 antagonist is about 1×10⁻⁷. In some embodiments, the daily dose of the LFA-1 antagonist is about 1×10⁻⁵ g. In some embodiments, the daily dose of the LFA-1 antagonist is about 1×10⁻³ g. In some embodiments, the daily dose of the LFA-1 antagonist is about 1×10⁻² g. In some embodiments the individual dose ranges from about 1×10⁻¹⁰ g to 5.0 g, or about 1×10⁻¹⁰ g to 2.5 g, or about 1×10⁻¹⁰ g to 1.00 g, or about 1×10⁻¹⁰ g to 0.5 g, or about 1×10−10 g to 0.25 g, or about 1×10⁻¹⁰ g to 0.1 g, or about 1×10⁻¹⁰ g to 0.05 g, or about 1×10⁻¹⁰ g to 0.025 g, or about 1×10⁻¹⁰ g to 1×10⁻² g, or about 1×10⁻¹⁰ g to 5×10⁻³ g, or about 1×10⁻¹⁰ g to 2.5×10⁻³ g, or about 1×10⁻¹⁰ g to 1×10⁻³ g, or about 1×10⁻¹⁰ g to 5×10⁻⁴ g, or 1×10⁻¹⁰ g to 2.5×10⁻⁴ g, or about 1×10⁻¹⁰ g to 1×10⁻⁴ g, or about 1×10⁻¹⁰ g to 5×10⁻⁵ g, or 1×10⁻¹⁰ g to 2.5×10⁻⁵ g, or about 1×10⁻¹⁰ g to 1×10⁻⁵ g, or about 1×10⁻¹⁰ g to 5×10⁻⁶ g, or about 1×10⁻⁹ g to 1.00 g, or about 1×10⁻⁹ g to 0.5 g, or about 1×10⁻⁹ g to 0.25 g, or about 1×10⁻⁹ g to 0.1 g, or about 1×10⁻⁹ g to 0.05 g, or about 1×10⁻⁹ g to 0.025 g, or about 1×10⁻⁹ g to 1×10⁻² g, or about 1×10⁻⁹ g to 5×10⁻³ g, or about 1×10⁻⁹ g to 2.5×10⁻³ g, or about 1×10⁻⁹ g to 1×10⁻³ g, or about 1×10⁻⁹ g to 5×10⁻⁴ g, or about 1×10⁻⁸ g to 5.0 g, or about 1×10⁻³ g to 2.5 g, or about 1×10⁻⁸ g to 1 g, or about 1×10¹³ g to 0.5 g, or about 1×10⁻⁸ g to 0.25 g, or about 1×10⁻⁸ g to 0.1 g, or about 1×10⁻³ g to 5×10⁻² g, or about 1×10⁻⁸ to 5×10⁻² g, or about 1×10⁻³ g to 2.5×10⁻² g, or about 1×10⁻⁸ g to 1×10⁻² g, or about 1×10⁻⁸ g to 5×10⁻³ g, or about 1×10⁻⁸ to 2.5×10⁻³ g, or about 1×10⁻⁸ g to 1×10⁻³ g, or about 1×10⁻⁸ g to 5×10⁻⁴ g, or about 1×10⁻⁷ g to 5.0 g, or about 1×10⁻⁷ g to 2.5 g, or about 1×10⁻⁷ g to 1 g, or about 1×10⁻⁷ g to 0.5 g, or about 1×10⁻⁷ g to 0.25 g, or about 1×10⁻⁷ g to 0.1 g, or about 1×10⁻⁷ g to 5×10⁻² g, or about 1×10⁻⁷ to 5×10⁻² g, or about 1×10⁻⁷ g to 2.5×10⁻² g, or about 1×10⁻⁷ g to 1×10⁻² g, or about 1×10⁻⁷ g to 5×10⁻³ g, or about 1×10⁻⁷ to 2.5×10⁻³ g, or about 1×10⁻⁷ g to 1×10⁻³ g, or about 1×10⁻⁷ to 5×10⁻⁴ g, or about 1×10⁻⁶ g to 5.0 g, or about 1×10⁻⁶ g to 2.5 g, or about 1×10⁻⁶ g to 1 g, or about 1×10⁻⁶ g to 0.5 g, or about 1×10⁻⁶ g to 0.25 g, or about 1×10⁻⁶ g to 0.1 g, or about 1×10⁻⁶ g to 5×10⁻² g, or about 1×10⁻⁶ to 5×10⁻² g, or about 1×10⁻⁴ g to 2.5×10⁻² g, or about 1×10⁻⁶ g to 1×10⁻² g, or about 1×10⁻⁶ g to 5×10⁻³ g, or about 1×10⁻⁶ to 2.5×10⁻³ g, or about 1×10⁻⁶ g to 1×10⁻³ g, or about 1×10⁻⁶ g to 5×10⁻⁴ g, or about 1×10⁻⁵ g to 5 g, or about 1×10⁻⁵ g to 2.5 g, or about 1×10⁻⁵ g to 1 g, or about 1×10⁻⁵ g to 0.5 g, or about 1×10⁻⁵ g to 0.25 g, or about 1×10⁻⁵ g to 0.1 g, or about 1×10⁻⁵ g to 0.05 g, or about 1×10⁻⁵ g to 2.5×10⁻² g, or about 1×10⁻⁵ g to 1×10⁻² g, or about 1×10⁻⁵ g to 5×10⁻³ g, or about 1×10⁻⁵ g to 2.5×10⁻³ g, or about 1×10⁻⁵ g to 1×10⁻³ g, or about 1×10⁻⁵ g to 5×10⁻⁴ g.

In some embodiments, the individual doses as described above, is repeated 1, 2, 3, 4, 5, 6, 7, 8, 9, or 10 times per day.

For other forms of administration, the daily dosages may range about the range described for systemic administration or may range about the range described for topical administration.

For slow or sustained release devices and formulations, in some embodiments, a typical dose range is about 0.1 mg to about 100 mg of the LFA-1 antagonist, released over the dosing period. In other embodiments, about 1 mg to about 50 mg, about 1 to about 25 mg, about 5 mg to about 100 mg, about 5 to about 50 mg, about 5 to about 25 mg, about 10 mg to about 100 mg, about 10 mg to about 50 mg, about 10 mg to about 25 mg, or about 15 mg to about 50 mg is released over the dosing period. The dosing period for slow release devices and formulations, typically range from about 10 days to about 1 year, about 30 days to about 1 year, about 60 days to about 1 year, about 3 months to about 1 year, about 4 months to about 1 year, about 5 months to about 1 year, or about 6 months to about 1 year. In some embodiments, the slow release devices and formulations release the LFA-1 antagonist, over the period of about 1 month to about 9 months, about 1 month to about 8 months, about 1 month to about 7 months, about 1 month, to about 6 months, about 1 month to about 5 months, about 1 month to about 4 months, or about 1 month to about 3 months. In other embodiments the slow release formulations and devices release the LFA-1 antagonist, for up to 1 month, 2 months, 3 months, 4 months, 5 months, 6 months, 7 months, 8 months, 9 months, 10 months, 12 months, 18 months, 2 years, 30 months, or 3 years. In some embodiments, the slow or sustained release device is a pump. In some embodiments, the slow or sustained release device is an implantable device. In some slow or sustained release formulations, it is a gel. In some slow or sustained release formulations, it is a biocompatible solid. In some slow or sustained release formulations, it is a biodegradable solid.

In some embodiments of the invention, the sustained release formulation and/or implantations release sufficient therapeutic agent to sustain a local level of the LFA-1 antagonist, of at least about 1 0 nM, about 50 nM, about 100 nM, about 150 nM, about 200 nM, about 250 nM, about 300 nM, about 350 nM, about 500 nM, about 600 nM, about 700 nM, about 800 nM, about 900 nM, about 1 μM, about 2 μM, about 3 μM, about 5 μM, about 6 μM, about 7 μM, about 8 μM, about 9 μM, about 10 μM, about 15 μM, about 20 μM, about 25 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, or about 100 μM across 1 year. In some embodiments of the invention, the sustained release formulation and/or implantations release sufficient therapeutic agent into a gastrointestinal tissue to sustain a local level of the LFA-1 antagonist, of at least about 10 nM, about 50 nM, about 100 nM, about 150 nM, about 200 nM, about 250 nM, about 300 nM, about 350 nM, about 500 nM, about 600 nM, about 700 nM, about 800 nM, about 900 nM, about 1 μM, about 2 μM, about 3 μM, about 5 μM, about 6 μM, about 7 μM, about 8 μM, about 9 μM, about 10 μM, about 15 μM, about 20 μM, about 25 CM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, or about 100 μM across 6 months.

The compositions of the invention may be packaged in multidose form or may be packaged in single dose units. Preservatives may be desirable to prevent microbial contamination during use. The composition of the invention can be formulated as a sterile unit dose type containing no preservatives. Alternatively, preservatives may be used.

Suitable preservatives for the compositions of the invention include: benzalkonium chloride, purite, peroxides, perborates, thimerosal, chlorobutanol, methyl paraben, propyl paraben, phenylethyl alcohol, edetate disodium, sorbic acid, Onamer M, or other agents known to those skilled in the art. In some embodiments of the invention, such preservatives may be employed at a level of from about 0.004% to about 0.02%. In some compositions of the present application the preservative benzalkonium chloride, may be employed at a level of from about 0.001% to less than about 0.01%, e.g. from about 0.001% to about 0.008%, or about 0.005% by weight. It has been found that a concentration of benzalkonium chloride of about 0.005% may be sufficient to preserve the compositions of the present invention from microbial attack. One of skill in the art can determine the proper concentration of ingredients as well as combinations of various ingredients for generating a suitable topical formulation. For example, ophthalmic drops or formulations for application to skin may use a mixture of methyl and propyl parabens at about 0.02 and about 0.04% respectively.

### Treatment of Inflammatory and Immune Related Conditions

The LFA-1 antagonists of the present invention may be used to treat a variety of inflammatory and immune related diseases and disorders, as LFA-1 has been implicated in a number of these disorders. Not intending to limit the mechanism of action, the methods of the present invention involve the inhibition of initiation and progression of inflammation related disease by inhibiting the interaction between LFA-1 and ICAM-1. LFA-1 and ICAM-1 are molecules with extracellular receptor domains which are involved in the process of lymphocyte/leukocyte migration and proliferation, leading to a cascade of inflammatory responses. Local administration of LFA-1 antagonists may be particularly effective in disease states where systemic administration of anti-LFA-1 monoclonal antibodies has proven effective, for example, as used in Raptiva clinical trials or in www.clinicaltrials.gov. Methods of the present invention provide anti-inflammatory effects, as described in more detail below, and are useful in the treatment of inflammation mediated diseases, for example, inflammatory bowel disease (IBD).

Human blood contains white blood cells (leukocytes) which are further classified as neutrophils, lymphocytes (with B- and T-subtypes), monocytes, eosinophils, and basophils. Several of these classes of leukocytes, neutrophils, eosinophils, basophils and lymphocytes, are involved in inflammatory disorders. LFA-1 is one of a group of leukointegrins which are expressed on most leukocytes, and is considered to be the lymphoid integrin which interacts with a number of ICAMs as ligands. Disrupting these interactions, and thus the immune/inflammatory response provides for reduction of inflammation in diseases or disorders such as IBD. For example, ICAM-1 (CD54) is a member of the ICAM family of adhesion receptors (ICAM-1, ICAM-2, ICAM-3, ICAM-4) in the immunoglobulin protein super family, and is expressed on activated leukocytes, dermal fibroblasts, and endothelial cells. See Krensky, A.M.; Sanchez-Madrid, F.; Robbins, E.; Nagy, J. A.; Springer, T. A. Burakoff, S. J. “The functional significance, distribution, and structure of LFA-1, LFA-2, and LFA-3: cell surface antigens associated with CTL-target interactions.” 1983 J. Immunol. 131, 611-616. It is normally expressed on the endothelial cells lining the vasculature, and is upregulated upon exposure to cytokines or compounds which induce cytokine release such as IL-1, LPS, SEB, and TNF during immune/inflammatory initiation.

Research conducted over the last decade has helped elucidate the molecular events involved in the movement and activation of cells in the immune system, focusing on cell-to-cell triggering interactions within the cascade. See Springer, T. A. “Adhesion receptors of the immune system.” Nature, 1990, 346, 425-434. The interaction of Intercellular Adhesion Molecules (ICAMs) with leukointegrins plays a role in the functioning of the immune system. It is believed that immune processes such as antigen presentation, T-cell mediated cytotoxicity and leukocyte transendothelial migration (diapedesis) require cellular adhesion mediated by ICAMs interacting with leukointegrins. See Kishimoto, T. K.; Rothlein; R. R. “Integrins, ICAMs, and selectins: role and regulation of adhesion molecules in neutrophil recruitment to inflammatory sites.” Adv. Pharmacol. 1994, 25, 117-138 and Diamond, M.; Springer, T. A. “The dynamic regulation of integrin adhesiveness.” Current Biology, 1994, 4, 506-532.

The interaction of ICAM-1 and LFA-1 (also referred to as α_{L}β₂ and CD11a/CD18) has been shown to be involved in the processes of adhesion, leukocyte transendothelial migration, migration to sites of injury, and proliferation of lymphocytes at the activated target site. For example, it is presently believed that prior to leukocyte transendothelial migration, a component of the inflammatory response, the presence of cytokines/chemokines activate integrins constitutively expressed on leukocytes. Blood vessel endothelial cells also upregulate ICAM-1 in response to the presence of the same cytokines/chemokines. As rolling leukocytes approach activated endothelial cells, their progress is first slowed by these upregulated ICAM-1 receptors. This is followed by a ligand/receptor interaction between LFA-1 and ICAM-1, expressed on blood vessel endothelial cell surfaces, which arrests the lymphocyte from rolling further. The lymphocyte then flattens, and transvasation takes place. This process is of importance both in lymphocyte transmigration through vascular endothelial as well as lymphocyte trafficking from peripheral blood to lymph nodes.

LFA-1 plays a role in creating and maintaining the immunological synapse, which may be defined as the physical structure of the interacting surfaces of T cells and Antigen Presenting Cells (APCs). LFA-1 stabilizes T-cell engagement with the APC, and thus leads to activation of T cells. The interaction of LFA-1 and ICAM-1 also appears to provide co-stimulatory signals to resting T cells. CD4+ T-cell proliferation and cytokine synthesis are mediated by this interaction as part of the inflammatory response.

Given the role that the interaction of ICAM-1 and LFA-1 plays in immune/inflammatory response, it is desirable to modulate these interactions to achieve a desired therapeutic result (e.g., inhibition of the interaction in the event of an overactive inflammatory response). The antagonism of the interaction between ICAMs and leukointegrins can be realized by agents directed against either component, for example with antibodies. Also, since LFA-1 has several ligand partners within the ICAM family (ICAM-1, ICAM-2 and ICAM-3), involving a number of signaling pathways, in some embodiments of the invention, it is desirable to modulate these interactions selectively.

The methods and compositions described herein can modulate one or more components of the pathways described herein. In addition to inhibiting interaction between LFA-1 and ICAM-1, the methods and compositions of the present invention may also intervene in either earlier or later portions of the inflammatory process as well. For example, upregulation of ICAM-1 or LFA-1 (activation) on endothelial cells or leukocytes, prior to tethering and transendothelial migration, may be modulated by the methods and compositions described herein. The present invention may be useful in modulating the expression of cytokines or chemokines that activate ICAM-1 and LFA-1 in the course of leukocyte trafficking, in modulating the transport of the cytokines or chemokines, in preventing transvasation of the arrested leukocyte, in modulating signalling via other mechanisms that are involved in leukocyte proliferation at the site of injury or inflammation, and the like.

The compositions and methods of the present invention are useful for treating inflammatory or immune related disorders and symptoms of the gastrointestinal system, including, but not limited to, inflammatory diseases such as inflammatory bowel disease, Crohn's disease or ulcerative colitis, and oral lichen planus. The compositions and formulations described herein are useful in treating gastrointestinal inflammation, such as inflammation of the mucosal layer of the gastrointestinal tract. The mucosal layer of the gastrointestinal tract includes the mucosa of the bowel (including the small intestine and large intestine), rectum, stomach (gastric) lining, oral cavity, and the like. Acute and chronic inflammatory conditions may be treated. Acute inflammation is generally characterized by a short time of onset and infiltration or influx of neutrophils. Chronic inflammation is generally characterized by a relatively longer period of onset (e.g., from several days, weeks, months, or years and up to the life of the subject), and infiltration or influx of mononuclear cells. Chronic inflammation can also typically characterized by periods of spontaneous remission and spontaneous occurrence. Thus, subjects with chronic gastrointestinal inflammation may be expected to require a long period of supervision, observation, or care.

Chronic gastrointestinal inflammatory conditions, also referred to as chronic gastrointestinal inflammatory diseases, having such chronic inflammation include, but are not necessarily limited to, inflammatory bowel disease, colitis induced by environmental insults (e.g., gastrointestinal inflammation (e.g., colitis) caused by or associated with (e.g., as a side effect) a therapeutic regimen, such as administration of chemotherapy, radiation therapy, and the like), colitis in conditions such as chronic granulomatous disease (Schappi et al., Arch. Dis. Child., 1984:147 (2001)), celiac disease, celiac sprue (a heritable disease in which the intestinal lining is inflamed in response to the ingestion of a protein known as gluten), food allergies, gastritis, infectious gastritis or enterocolitis (e.g., Helicobacter pylori-infected chronic active gastritis) and other forms of gastrointestinal inflammation caused by an infectious agent, and other like conditions. The acute and chronic inflammation is thought to be, without being bound by theory, secondary to an increase in pro-inflammatory cytokines (particularly tumor necrosis factor-alpha) and an increase in epithelial cell apoptosis. The resultant manifestations of these factors are thought to be, without being limited by theory, a loss of the mucosal epithelial lining and the above stated neutrophil/monocyte infiltrate.

The present invention is useful in treating inflammatory bowel disease, or IBD. IBD refers to any of a variety of diseases typically characterized by inflammation of all or part of the intestines. Examples of inflammatory bowel disease include, but are not limited to, Crohn's disease, ulcerative colitis, irritable bowel syndrome, mucositis, radiation induced enteritis, short bowel syndrome, celiac disease, colitis, stomach ulcers, diverticulitis, pouchitis, proctitis, and chronic diarrhea. Reference to IBD is exemplary of gastrointestinal inflammatory conditions, and is not meant to be limiting.

Symptoms of IBD can include, but not be limited to, symptoms such as abdominal pain, diarrhea, rectal bleeding, weight loss, fever, loss of appetite, and other more serious complications, such as dehydration, anemia and malnutrition. A number of such symptoms are subject to quantitative analysis (e.g. weight loss, fever, anemia, etc.). Some symptoms are readily determined from a blood test (e.g. anemia) or a test that detects the presence of blood (e.g. rectal bleeding). Treatment of IBD with LFA-1 antagonists described here in can reduce symptoms, which can be a qualitative or quantitative reduction in detectable symptoms, including but not limited to, a detectable impact on the rate of recovery from disease (e.g. rate of weight gain). The diagnosis may be determined by way of an endoscopic observation of the mucosa, and pathologic examination of endoscopic biopsy specimens.

The formulations described herein can also be used to treat those at risk for IBD, which encompasses the segment of the world population that has an increased risk (i.e. over the average person) for IBD. IBD appears to be most common in the United States, England, and northern Europe, and is more common in certain subgroups of the populations, such as people of Jewish descent. An increased frequency of this condition has also been observed in developing nations. Increased risk is also typically prevalent in people with family members who suffer from inflammatory bowel disease. Thus, those at risk may also be treated with LFA-1 antagonists of the present invention.

Accordingly, in one aspect, a method is provided for treatment of an inflammatory or immune related disorder of one or more tissues of the gastrointestinal system in a subject comprising administering to said subject in need thereof a formulation comprising an LFA-1 antagonist or a pharmaceutically acceptable salt or ester thereof, and a pharmaceutically acceptable excipient, wherein the LFA-1 antagonist has a systemic clearance rate greater than about 2 mL/min/kg when administered to a subject. Administration may be oral or via suppository,

The benefits of oral administration include localized delivery of the therapeutic agent and minimal systemic side effects due to low systemic bioavailability. The LFA-1 antagonist is administered orally, but is delivered only in the GI tract where the formulation permits the drug to dissolve in GI fluid. The LFA-1 antagonist is then distributed to the surface of the GI mucosa, whereupon the LFA-1 antagonist penetrates through intestinal epithelium to local adjacent tissue. The fluids in the GI tract having high levels of drug will travel down the GI tract with normal GI motility and gastric flow and coat the effected surface of GI along the way. Additionally, LFA-1 antagonist that does distribute out of local intestinal tissue and into the vasculature is swept to the liver and delivered via bile into the lower GI tract. Suitable formulations and additional carriers are discussed herein and, additionally, described in Remington “The Science and Practice of Pharmacy” (20^{th} Ed., Lippincott Williams & Wilkins, Baltimore Md.), the teachings of which are incorporated by reference in their entirety herein.

In some embodiments, therapeutic agents of the invention have a rapid systemic clearance such that any drug that gets absorbed systemically is quickly cleared. In some embodiments, the LFA-1 antagonist may have a systemic clearance rate of greater than about 1 mL/min/kg, about 2 mL/min/kg, about 3 mL/min/kg, about 4 mL/min/kg, about 5 mL/min/kg, about 6 mL/min/kg, about 7 mL/min/kg, about 8 mL/min/kg, about 9 mL/min/kg, about 10 mL/min/kg, about 11 mL/min/kg, about 12 mL/min/kg, about 13 mL/min/kg, about 14 mL/min/kg, about 15 mL/min/kg, about 16 mL/min/kg, about 17 mL/min/kg, about 18 mL/min/kg, about 19 mL/min/kg, about 20 mL/min/kg, about 25 mL/min/kg, about 30 mL/min/kg, about 35 mL/min/kg, about 40 mL/min/kg, about 45 mL/min/kg, about 50 mL/min/kg, about 60 mL/min/kg, about 65 mL/min/kg, about 70 mL/min/kg, about 75 mL/min/kg, about 80 mL/min/kg, about 85 mL/min/kg, about 90 mL/min/kg, about 95 mL/min/kg, or about 100 mL/min/kg.

It is known that LFA-1 interacts with several ligands which could result in several unwanted side effects. Thus in some embodiments, the local concentration of therapeutic agent is about 2×, 3×, 4×, 5×, 10×, 25×, 50×, or about 100× greater than the systemic concentration. In another embodiment of the current invention, local concentration of LFA-1 antagonist is 1000× greater than the systemic concentration. In one embodiment, the local concentration is about 10,000× or more greater than the systemic concentration at the same time point. The concentration of therapeutic agent may be measured using any known method in the art. For example, radiolabelled therapeutic drug may be used and measurements taken from the local site of administration compared to systemic levels (e.g. plasma level concentrations).

The compositions may be delivered with a pharmacokinetic profile that results in the delivery of an effective dose of the LFA-1 antagonist. The actual effective amounts of drug can vary according to the specific drug or combination thereof being utilized, the particular composition formulated, the mode of administration, and the age, weight, condition of the patient, and severity of the symptoms or condition being treated. Dosages for a particular patient can be determined by one of ordinary skill in the art using conventional considerations, (e.g. by means of an appropriate, conventional pharmacological protocol).

Once released in the environment of the gastrointestinal mucosa, the LFA-1 antagonist is absorbed locally. In some embodiments, the LFA-1 antagonist achieves a local tissue concentration of greater than about 1 μM within about 4 hours following administration to a subject. In other embodiments, the LFA-1 antagonist achieves a local tissue concentration of greater than about 1 μM within about 3 hours following administration to a subject. In other embodiments, the LFA-1 antagonist achieves a local tissue concentration of greater than about 1 μM within about 2 hours following administration to a subject. In other embodiments, the LFA-1 antagonist achieves a local tissue concentration of greater than about 1 μM within about 1 hour following administration to a subject. In other embodiments, the LFA-1 antagonist achieves a local tissue concentration of greater than about 1 μM within about 50 min, about 40 min, about 30 min, about 20 min, about 10 min, about 5 min, or about 3 minutes following administration to a subject.

After the formulation of the invention is orally administered as described above, the LFA-1 antagonist is released in the GI tract and is present in a therapeutically effective concentration within about 1 mm of an epithelial surface to which the LFA-1 antagonist is distributed from the GI tract. In some embodiments, the LFA-1 antagonist is present in a therapeutically effective concentration within about 2 mm, about 3 mm, about 4 mm, about 5 mm, about 6 mm, about 7 mm, about 8 mm, about 9 mm, about 10 mm, about 12 mm, about 14 mm, about 16 mm, about 18 mm, about 20 mm, about 30 mm, about 40 mm, or about 50 mm of an epithelial surface to which the LFA-1 antagonist is distributed from the GI tract. In embodiments, wherein the formulations of the invention are administered to the GI tract by suppository, the LFA-1 antagonist is released in the GI tract and is present in a therapeutically effective concentration within about 1 mm of an epithelial surface to which the LFA-1 antagonist is distributed once released in the GI tract.

In some embodiments, the LFA-1 antagonist has a local tissue concentration of greater than about 10 nM within about 4 hours following administration to the subject. In other embodiments, the LFA-1 antagonist has a local tissue concentration of greater than about 20 nM, about 30 nM, about 40 nM, about 50 nM, about 75 nM, about 100 nM, about 150 nM, about 200 nM, about 150 nM, about 300 nM, about 400 nM, about 500 nM, about 600 nM, about 700 nM, about 800 nM, about 900 nM, about 1 μM, about 2 μM, about 3 μM, about 4 μM, about 5 μM, about 6M, about 7 μM, about 8 μM, about 9 μM, or about 10 μM within about 4 hours following administration to the subject, In yet other embodiments, the LFA-1 antagonist has a local tissue concentration of greater than about than about 10 nM, about 20 nM, about 30 nM, about 40 nM, about 50 nM, about 75 nM, about 100 nM, about 150 nM, about 200 nM, about 150 nM, about 300 nM, about 400 nM, about 500 nM, about 600 nM, about 700 nM, about 800 nM, about 900 nM, about 1 μm, about 2 M, about 3 μM, about 4 μM, about 5 μM, about 6 μM, about 7 μM, about 8 μM, about 9 μM, or about 10 μM within about 5 hours following administration to the subject. The invention also provides methods wherein the LFA-1 antagonist has a local tissue concentration of greater than about than about 10 nM, about 20 nM, about 30 NM, about 40 nM, about 50 nM, about 75 nM, about 100 nM, about 150 nM, about 200 nM, about 150 nM, about 300 nM, about 400 nM, about 500 nM, about 600 nM, about 700 nM, about 800 nM, about 900 nM, about 1 μM, about 2 μM, about 3 μM, about 4 μM, about 5 μM about 6 μm, about 7 μM, about 8 μM, about 9 μM, or about 10 μM within about 3 hours following administration to the subject. The LFA-1 antagonist may also have a local tissue concentration of greater than about than about 10 nM, about 20 nM, about 30 nM, about 40 nM, about 50 nM, about 75 nM, about 100 nM, about 150 nM, about 200 nM, about 150 nM, about 300 nM, about 400 nM, about 500 nM, about 600 nM, about 700 nM, about 800 nM, about 900 nM, about 1 μM, about 2 μM, about 3 μM, about 4 μM, about 5 μM, about 6 μM, about 7™, about 8;M, about 9 μM, or about 10 μM within about 2 hours following administration to the subject. In other embodiments, the LFA-1 antagonist has a local tissue concentration of greater than about than about 10 nM, about 20 nM, about 30 nM, about 40 nM, about 50 nM, about 75 nM, about 100 nM, about 150 nM, about 200 nM, about 150 nM, about 300 nM, about 400 nM, about 500 nM, about 600 nM, about 700 nM, about 800 nM, about 900 nM, about 1 μM, about 2 μM, about 3 μM, about 4 M, about 5 μM, about 6 μM, about 7 μM, about 8 μM, about 9 μM, or about 10 μM within about 1 hour following administration to the subject. In some other embodiments, the LFA-1 antagonist has a local tissue concentration of greater than about than about 10 nM, about 20 nM, about 30 nM, about 40 nM, about 50 nM, about 75 nM, about 100 nM, about 150 nM, about 200 nM, about 150 nM, about 300 nM, about 400 nM, about 500 nM, about 600 nM, about 700 nM, about 800 nM, about 900 nM, about 1 μM, about 2 μM, about 3 μM, about 4 μM, about 5 μM, about 6 μM, about 7 μM, about 8 μM, about 9 μM, or about 10 μM within about 50 min, about 40 min, about 30 min, about 20 min, about 10 min, about 9 min, about 8 min, about 7 min, about 6 min, about 5 min, about 4 min, about 3 min, about 2 min, or about 1 min following administration to the subject.

In some of the methods of the invention, the LFA-1 antagonist maintains a local tissue concentration of greater than about 10 nM for at least about 8 hours following administration. In other embodiments, the LFA-1 antagonist maintains a local tissue concentration of greater than about 10 nM, about 20 nM, about 30 nM, about 40 nM, about 50 nM, about 75 nM, about 100 nM, about 150 nM, about 200 nM, about 150 nM, about 300 nM, about 400 nM, about 500 nM, about 600 nM, about 700 nM, about 800 nM, about 900 nM, or about 1 μM, for at least about 8 hours following administration. In other embodiments, the LFA-1 antagonist maintains a local tissue concentration of greater than about 10 nM, about 20 nM, about 30 nM, about 40 nM, about 50 nM, about 75 nM, about 100 nM, about 150 nM, about 200 mM, about 150 mM, about 300 nM, about 400 nM, about 500 nM, about 600 nM, about 700 nM, about 800 nM, about 900 nM, or about 1 μM, for at least about 10 hours, about 9 hours, about 8 hours, about 7 hours, about 6 hours, about 5 hours, about 4 hours, about 3 hours, about 2 hours, or about 1 hour following administration.

In some of the methods of the invention, the LFA-1 antagonist has a local tissue concentration of greater than about 1 μM and a systemic concentration as measured in plasma of less than about 100 nM, within about 4 hrs following administration. In other embodiments, the LFA-1 antagonist has a local tissue concentration of greater than about 1 μM and a systemic concentration as measured in plasma of less than about 80 nM, about 70 nM, about 60 or about 50 nM, within about 4 hours, about 3 hours, about 2 hours, about 1 hour, about 50 min, about 40 min, about 30 min, about 20 min, about 10 min, or about 5 min following administration.

Additionally, in some of the methods of the invention, the LFA-1 antagonist is present in a therapeutically effective concentration within about 1 mm of an epithelial surface to which the formulation is applied and is present in blood plasma below a therapeutically effective level, within about 4 hrs following administration. In other embodiments, the LFA-1 antagonist is present in a therapeutically effective concentration within about 2 mm, about 3 mm, about 4 mm, about 5 mm, about 6 mm, about 7 mm, about 8 mm, about 9 mm, about 10 mm, about 12 mm, about 14 mm, about 16 mm, about 18 mm, about 20 mm, about 30 mm, about 40 mm, or about 50 mm of an epithelial surface to which the formulation is applied and is present in blood plasma below a therapeutically effective level, within about 4 hrs following administration. Alternatively, the LFA-1 antagonist may be present in a therapeutically effective concentration within about 1 mm of an epithelial surface to which the formulation is applied and is present in blood plasma below a therapeutically effective level, within about 6 hours, about 5 hours, about 3 hours, about 2 hours, about 1 hour, about 50 min, about 40 min, about 30 min, about 20 min, about 10 min or about 5 min following administration.

Treatment of the conditions may include co-administration of the LFA-1 antagonist formulations, depending on the type of condition being treated. For example, when the condition being treated is an inflammatory bowel disease, the additional agent can be steroid, such as a corticosteroid, or other type of immunosuppressive agent. The additional agents to be co-administered, such as immunosuppressive agents or corticosteroids can be any of the well-known agents in the art, including, but not limited to, those that are currently in clinical use. Examples include antibodies (see for example, U.S. Patent Application No. 20070224191 and 20050019323) or compounds such as limonene (U.S. Patent Application No. 20030199592). The additional agents used may be 5-aminosalicylates (5-ASA) compounds, such as sulfasalzine (Azulfidine), osalazine (Dipentum), and mesalamine (examples include Pentasa, Asacol, Dipentum, Colazal, Rowasa enema, and Canasa suppository). Corticosteroids, such as prednisone, and others, such as those that act systemically, may also be used. For example, topical corticosteroids, like budesonide can be used. Antibiotics such as metronidazole (Flagyl) and ciprofloxacin (Cipro) can also be used. Other examples of additional agents include immunomodulators such as 6-mercaptopurine (6-MP), azathioprine (Imuran), methotrexate (Rheumatrex, Trexall), infliximab (Remicade), and adalimumab (Humira). Additional agents used may be calcineurin inhibitors such as cyclosporine, tacrolimus, pimecrolimus and sirolimus.

In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent. For example, combinations of agents can be used to treat LFA-1 mediated disorders or to modulate the side-effects of one or more agents in the combination. As in some instances, pathological events in a disease state are marked by a combination of impaired autoregulation, apoptosis, ischemia, neovascularization, and/or inflammatory stimuli, it may be desirable to administer the LFA-1 antagonists of the invention in combination with other therapeutic agents to additionally or synergistically intervene. To intervene, without being bound by theory, the second therapeutic agent can be an antioxidant, antiinflammatory agent, antimicrobial including antibacterial, antiviral and antifungal agents, antiangiogenic agent, anti-apoptotic agent, or combinations thereof. In some embodiments of the invention, in addition to administering a compound which directly competes for binding to LFA-1, an additional therapeutic agent may be administered which is an allosteric, but not a directly competitive, antagonist of LFA-1 as discussed above, potentially resulting in synergistic efficacy. An example of such allosteric antagonist is the class of hydantoin inhibitors of LFA-1. (See for example, Keating et al., Protein Science, 15, 290-303, (2006)). Another class of therapeutic agents which may be useful to administer in combination, prior to, after, or concomitantly with the LFA-1 antagonists of the invention is the group of drugs which inhibit Vascular Endothelial Growth Factor, and thus may target another route of initiation of neovascularization, as inflammation, without being limited by theory, is typically induced by the process of leukocyte adhesion and neovascularization. Any VEGF inhibitor may be of use in the compositions of the invention for example, inhibitors chosen from: 1) neutralizing monoclonal antibodies against VEGF or its receptor, 2) small molecule tyrosine kinase inhibitors of VEGF receptors, 3) soluble VEGF receptors which act as decoy receptors for VEGF, and 4) ribozymes which specifically target VEGF, or combinations thereof. Some examples of antibodies which are active against VEGF are, for example, e.g., Lucentis (ranibizumab), and Avastin (bevacizumab). An example of an oligonucleotide drug is, e.g., Macugen (pegaptanib sodium injection). Small molecule tyrosine kinase inhibitors include, for example, pazopanib, sorafenib, sutent, and the like can also be used.

Similarly, additional agents can be chosen from calcineurin inhibitors such as cyclosporine, or cyclosporine-related drugs, including but not limited to members of the cyclosporine family, and other related calcineurin antagonists including sirolimus, tacrolimus and pimecrolimus. Other anti-inflammatory agents may be administered in combination, prior to, after, or concomitantly with the LFA-1 antagonists of the invention. The anti-inflammatory agents can be chosen from corticosteroid related drugs including but not limited to dexamethasone, fluoromethalone, medrysone, betamethasone, triamcinolone, triamcinolone acetonide, prednisone, prednisolone, hydrocortisone, rimexolone, and pharmaceutically acceptable salts thereof, prednicarbate, deflazacort, halomethasone, tixocortol, prednylidene, prednival, paramethasone, methylprednisolone, meprednisone, mazipredone, isoflupredone, halopredone acetate, halcinonide, formocortal, flurandrenolide, fluprednisolone, fluprednidine acetate, fluperolone acetate, fluocortolone, fluocortin butyl, fluocinonide, fluocinolone acetonide, flunisolide, flumethasone, fludrocortisone, fluclorinide, enoxolone, difluprednate, diflucortolone, diflorasone diacetate, desoximetasone (desoxymethasone), desonide, descinolone, cortivazol, corticosterone, cortisone, cloprednol, clocortolone, clobetasone, clobetasol, chloroprednisone, cafestol, budesonide, beclomethasone, amcinonide, allopregnane acetonide, alclometasone, 21-acetoxypregnenolone, tralonide, diflorasone acetate, deacylcortivazol, RU-26988, budesonide, deacylcortivazol, and the like. Alternatively, the antiinflammatory agents can be an NSAIDs including but not limited to acetaminophen, acemetacin, aceclofenac, alminoprofen, amfenac, bendazac, benoxaprofen, bromfenac, bucloxic acid, butibufen, carprofen, celecoxib, cinmetacin, clopirac, diclofenac, etodolac, etoricoxib, felbinac, fenclozic acid, fenbufen, fenoprofen, fentiazac, flunoxaprofen, flurbiprofen, ibufenac, ibuprofen, indomethacin, isofezolac, isoxicam, isoxepac, indoprofen, ketoprofen, lonazolac, loxoprofen, mefenamic acid, meclofenamic acid, meloxicam, metiazinic acid, mofezolac, miroprofen, naproxen, niflumic, oxaprozin, pirozolac, pirprofen, pranoprofen, protizinic acid, rofecoxib, salicylic acid and its derivatives (i.e. for example, aspirin), sulindac, suprofen, suxibuzone, triaprofenic acid, tolmetin, valdecoxib, xenbucin, ximoprofen, zaltoprofen, zomepirac, aspirin, acemetcin, bumadizon, carprofenac, clidanac, diflunisal, enfenamic acid, fendosal, flufenamic acid, flunixin, gentisic acid, ketorolac, mesalamine, prodrugs thereof, or the like.

Oxidative stress may be induced in cells with impaired autoregulatory and ischemic processes induced by LFA-1 mediated immune disorders. Therefore, anti-oxidants may be useful to administer in combination, prior to, after, or concomitantly with the LFA-1 antagonists of the invention. Examples of suitable anti-oxidants useful in the methods of the invention include, but are not limited to, ascorbic acid, tocopherols, tocotrienols, carotinoids, glutathione, α-lipoic acid, ubiquinols, bioflavonoids, carnitine, and superoxide dismutase mimetics, such as, for example, 2,2,6,6-tetramethyl-1-piperidinyloxy (TEMPO), DOXYL, PROXYL nitroxide compounds; 4-hydroxy-2,2,6,6-tetramethyl-1-piperidinyloxy (Tempol), M-40401, M-40403, M-40407, M-40419 μM-40484, M-40587, M-40588, or the like.

In some embodiments of the invention, methods are provided wherein anti-apoptotic therapeutic agents may be administered in combination, prior to, after, or concomitantly with the LFA-1 antagonists of the invention. Examples of suitable anti-apoptotic agents are, for example, inhibitors of caspases, cathepsins, and TNF-α.

Another class of therapeutic agents which may be useful to administer in combination, prior to, after, or concomitantly with the LFA-1 antagonists of the invention are antimicrobial agents. Suitable antimicrobial compounds, include, but are not limited to, penicillins, such as, for example, amoxicillin, ampicillin, azlocillin, carbenicillin, cloxacillin, dicloxacillin, flucloxacillin, mezlocillin, nafcillin, penicillin, piperacillin, ticarcillin, and the like; beta-lactamase inhibitors; carbapenems, such as, for example, ertapenem, imipenern, meropenem, and the like; cephalosporins, such as, for example, cefaclor, cefamandole, cefoxitin, cefprozil, cefuroxime, cefixime, cefdinir, cefditoren, cefoperazone, cefotaxime, cefpodoxime, cefadroxil, ceftazidime, ceftibuten, ceftizoxime, ceffiriaxone, cefazolin, cefixime, cephalexin, cefepime, and the like; quinolones, such as, for example, ciprofloxacin, enoxacin, gatifloxacin, levofloxacin, lomefloxacin, morifloxacin, norfloxacin, ofloxacin, trovafloxacin, and the like; macrolides, such as, for example, azithromycin, clarithromycin, dirithromycin, erythromycin, milbemycin, troleandomycin, and the like; monbactams, such as, for example, LFA-1 antagonist, and the like; tetracyclins, such as, for example, demeclocyclin, doxycycline, minocycline, oxytetracyclin, tetracycline, and the like; aminoglycosides, such as, for example, amikacin, gentamicin, kanamycin, neomycin, netilmicin, paromomycin, streptomycin, tobramycin, and the like; carbacephem, such as, for example, loracarbef, and the like; streptogramins; sulfonamides, such as, for example, mefanide, prontosil, sulfacetamide, sulfamethizole, sulfanilamide, sulfasalazine, sulfisoxazole, trimethoprim, trimethoprim-sultamethoxazole, and the like; and the combination drugs such as for example, sulfamethoxazole and trimethoprim, and the like; anti-virals, such as, for example, acyclovir, amantadine, combivir, docosanol, emtricitabine, foscamet, ganciclovir, gardasil, immunovir, indinavir, inosine, interferon, lopinavir, loviride, moroxydine, nevirapine, nexavir, penciclovir, pleconaril, ribavirin, rimantadine, ritonavir, tenofovir, trifluridine, viramidine, and zidovudine; antifungals, such as, for example, amphotericin B, miconazole, ketoconazole, clotrimazole, fluconazole, terbinafine, butenafine, anidulafungin, micafungin and tolnaftate; and polypeptides, such as, for example, bacitracin, colistin, polymyxin B, and the like.

Examples of other therapeutic agents which may be useful to administer in combination, prior to, after, or concomitantly with the LFA-1 antagonists of the invention are, include, but are not limited to: (a) anti-diabetic agents such as insulin and insulin mimetics, sulfonylureas (e.g., glyburide, meglinatide), biguanides, e.g., metformiin (Glucophage™), .alpha.-glucosidase inhibitors (acarbose), insulin sensitizers, e.g., thiazolidinone compounds, rosiglitazone (Avandia™), troglitazone (Rezulin™), ciglitazone, pioglitazone (Actos™) and englitazone; (b) cholesterol lowering agents such as HMG-CoA reductase inhibitors (e.g., lovastatin, simvastatin, pravastatin, fluvastatin, atorvastatin and other statins), bile acid sequestrants (e.g., cholestyramine and colestipol), vitamin B.sub.3 (also known as nicotinic acid, or niacin), vitamin B₆ (pyridoxine), vitamin B₁₂ (cyanocobalamin), fibric acid derivatives (e.g., gemfibrozil, clofibrate, fenofibrate and benzafibrate), probucol, nitroglycerin, and inhibitors of cholesterol absorption (e.g., beta-sitosterol and acylCoA-cholesterol acyltransferase (ACAT) inhibitors such as melinamide), HMG-CoA synthase inhibitors, squalene epoxidase inhibitors and squalene synthetase inhibitors; and (c) antithrombotic agents, such as thrombolytic agents (e.g., streptokinase, alteplase, anistreplase and reteplase), heparin, hirudin and warfarin derivatives, β-blockers (e.g., atenolol), β-adrenergic agonists (e.g., isoproterenol), ACE inhibitors and vasodilators (e.g., sodium nitroprusside, nicardipine hydrochloride, nitroglycerin and enaloprilat).

Examples of other therapeutic agents which may be useful to administer in combination, prior to, after, or concomitantly with LFA-1 antagonists are antiviral agents, which include, but are not limited to therapeutic agents such as entry inhibitors, reverse transcriptase inhibitors, nucleoside or nucleotide analogs, protease inhibitors, and inhibitors of viral release from host cells. Some illustrative therapeutic agents include, but are not limited to abacavir, acyclovir, adefovir, amantadine, amprenavir, arbidol, atazanavir, atripla, brivudine, cidofovir, combivir, darunavir, delavirdine, didanosine, docosanol, edoxudine, efavirenz, emtricitabine, enfuvirtide, entecavir, famciclovir, fomivirsen, foscamet, fosfonet, ganciclovir, gardasil, ibacitabine, immunovir, idoxuridine, imiquimod, indinavir, inosine, interferon type III, interferon type II, interferon type I, interferon, lamivudine, lopinavir, loviride, maraviroc, moroxydine, nelfimavir, neviapine, nexavir, oseltamivir, penciclovir, peramivir, pleconaril, podophyllotoxin, raltegravir, ribavirin, rimantadine, ritonavir, saquinavir, stavudine, tenofovir, tenofovir disoproxil, tipranavir, trifluridine, trizivir, tromantadine, truvada, valaciclovir, valganciclovir, vicriviroc, vidarabine, viramidine, zalcitabine, zanamivir, or zidovudine.

### EXAMPLES

### Example 1

### Human T-Cell Adhesion Assay

The T-cell adhesion assay was performed using the human T-lymphoid cell line HuT 78 (ATCC TIB-161). Goat anti-HuIgG(Fc) was diluted to 2 μg/ml in PBS and 96-well plates were coated with 50 μl/well at 37° C. for 1 h. Plates were washed with PBS and blocked for 1 h at room temperature with 1% BSA in PBS. 5 domain ICAM-Ig was diluted to 100 ng/ml in PBS and 50 μl/well was added to the plates O/N at 4° C. HuT 78 cells were centrifuged at 100 g and the cell pellet was treated with 5 mM EDTA for ˜5′ at 37° C. in a 5% CO₂ incubator. Cells were washed in 0.14 M NaCl, 0.02 M Hepes, 0.2% glucose and 0.1 mM MnCl₂ (assay buffer) and centrifuged. The cells were resuspended in assay buffer to 3.0×10⁶ c/ml. Inhibitors were diluted in assay buffer to a 2× final concentration and pre-incubated with HuT78 cells for 30′ at room temperature. 100 μl/well of cells and inhibitors were added to the plates and incubated at room temperature for 1 h. 100 μl/well PBS was added and the plates were sealed and centrifuged inverted at 100 g for 5′. Unattached cells were flicked out of the plate and excess PBS was blotted on a paper towel. 60 μl/well p-nitrophenyl n-acetyl-β-D-glucosaminide (0.257 g to 100 ml citrate buffer) was added to the plate and incubated for 1.5 h at 37° C. The enzyme reaction was stopped with 90 μl/well 50 mM glycine/5 mM EDTA and read on a platereader at 405 nM. HUT 78 cell adhesion to 5dICAM-Ig was measured using the p-nitrophenyl n-acetyl-β-D-glucosaminide method of Landegren, U. (1984). J. Immunol. Methods 57, 379-388. The results are shown in FIG. 1.

### Example 2

### LFA-1:ICAM-1 Receptor Binding Assay Using Forward Format Assay

Competitive inhibition of the LFA-1:ICAM-1 interaction is quantitated by adding known amounts of inhibitors.

Purified full length recombinant human LFA-1 protein is diluted to 2.5 μg/ml in 0.02 M Hepes, 0.15 M NaCl, and 1 mM MnCl₂ and 96-well plates (50 μl/well) are coated overnight at 4° C. The plates are washed with wash buffer (0.05% Tween in PBS) and blocked for 1 h at room temperature with 1% BSA in 0.02M Hepes, 0.15 M NaCl, and 1 mM MnCl₂. Plates are washed. 50 μl/well inhibitors, appropriately diluted in assay buffer (0.5% BSA in 0.02M Hepes, 0.1 5M NaCl, and 1 mM MnCl₂), are added to a 2× final concentration and incubated for 1 h at room temperature. 50 μl/well of purified recombinant human 5 domain ICAM-Ig, diluted to 50 ng/ml in assay buffer, is added and incubated 2 h at room temperature. Plates are washed and bound ICAM-Ig is detected with Goat anti-HuIgG(Fc)-HRP for 1 h at room temperature. Plates are washed and developed with 100 μl/well TMB substrate for 10-30′ at room temperature. Colorimetric development is stopped with 100 μl/well 1M H₂PO₄ and read at 450 nM on a platereader.

### Example 3

### In-Vitro Inhibition of Antigen Stimulated Release of Cytokines from Human Peripheral Blood Monocytes (PBMC)

One form of the LFA-1 antagonist of Formula I, Compound 12, was evaluated for its ability to inhibit release of inflammatory cytokines, in human mononuclear cells (PBMC) stimulated with staphylococcal enterotoxin B (SEB). Stock solutions of Compound 12, Rebamipide (a mucosal protective agent), and Cyclosporin A (CsA) were prepared in culture media and dilutions were prepared by addition of culture media to achieve the desired concentration. Negative controls were prepared without SEB stimulation. SEB stimulation with vehicle (0.25% DMSO/media) was used as the positive control.

Human PBMC, frozen in cryopreservation media were thawed, washed with RPMI culture media containing 10% FBS in growth media and seeded onto a 96 well plate at 20,000 cells/well containing 180 μl culture media. Cells were incubated in the presence of Compound 12, Rebamipide or CsA at 37° C. for 1 hour prior to stimulation with SEB. SEB was added at 1 ng/ml and cell supernatants were harvested at 6, 16, and 48 hours. Cytokine levels in the assay supernatants were determined using a Luminex multiplex assay.

Compound 12 demonstrated potent inhibition of the release of inflammatory cytokines, particularly the T-cell regulating cytokines, IL-2 and IL-4, with increasing dose. The results are shown in Tables 1, 2, and 3. Additionally, in vitro inhibition of IL-2 release for various LFA-1 antagonists is shown in FIG. 1. The pattern of cytokine release inhibited by more than 50% with Compound 12 is similar to that seen in comparison with CsA. The exceptions to this similarity include IL-3, Il-6, and IL-12p40.

TABLE 1
EC50 Concentrations for Inhibition of IL-2, IFNγ, MIP-1α, and TNF-α.

| | EC50 μM Cytokine Release | | | |
|---|---|---|---|---|
| | IL-2 | IFNγ | MIP-1α | TNF-α |
| Compound 12 | 0.0018 | 0.0016 | 0.020 | 0.076 |
| Rebamipide | >1000 | >1000 | >1000 | >1000 |
| Cyclosporine A | 0.00094 | 0.00050 | 0.0011 | 0.00049 |

TABLE 2
EC50 Concentrations for Inhibition of IL-4, IL-10, IP-10, GM-CSF and MCP-1.

| | EC50 μM Cytokine Release | | | | |
|---|---|---|---|---|---|
| | IL-4 | IL-10 | IP-10 | GM-CSF | MCP-1 |
| Compound 12 | 0.143 | 0.147 | 1.158 | 0.545 | 0.0050 |
| Rebamipide | >1000 | >1000 | >1000 | >1000 | >1000 |
| Cyclosporine A | 0.0063 | 0.0292 | 0.167 | 0.0202 | 0.0926 |

TABLE 3
EC50 Concentrations for Inhibition of IL-1α, IL-1β, IL-3, IL-5, IL-6, IL-12p40, and IL-13.

| | EC50 μM Cytokine Release | | | | | | |
|---|---|---|---|---|---|---|---|
| | IL-1α | IL-1β | IL-3 | IL-5 | IL-6 | IL-12p40 | IL-13 |
| Compound 12 | 0.24 | 0.36 | 52.23 | 0.11 | 43.51 | >1000 | 0.36 |
| Rebamipide | >1000 | >1000 | >1000 | >1000 | >1000 | >1000 | >1000 |
| Cyclosporine A | 0.002 | 0.003 | 0.002 | 0.073 | 0.001 | 0.002 | 0.074 |

### Example 4

### Formulations of LFA-1 Antagonist

One compound of Formula I (Compound 12) was formulated in several compositions for administration as gels, lotions, ointments, and solutions, for administration by varying routes, including but not limited to topical, via instillation, aerosol, transdermal patch, via insert, or oral administration.

TABLE 4
Gel Formulations 1 and 2 of Compound 12.

| Formulation 1 (% w/w) | Formulation 2 (% w/w) |
|---|---|
| 1% Form A of Compound 12 | 1% Form A of Compound 12 |
| 15% Dimethyl Isosorbide | 15% Dimethyl Isosorbide |
| 25% Transcutol | 25% Transcutol |
| 12% Hexylene glycol | 12% Hexylene glycol |
| 5% Propylene Glycol | 5% Propylene Glycol |
| 0.15% Methylparaben | 0.15% Methylparaben |
| 0.05% Propylparaben | 0.05% Propylparaben |
| 0.01% EDTA | 0.01% EDTA |
| 0.5% Penmulen TR-1 | 1% Hydroxyethyl Cellulose |
| q.s. pH 6.0 25% Trolamine | q.s. pH 4.5 25% Trolamine |
| q.s. 100 Water | q.s. 100 Water |

TABLE 5
Lotion Formulations 3 and 4 of Compound 12.

| Formulation 3 (% w/w) | Formulation 4 (% w/w) |
|---|---|
| 1% Form A | 1% Form A |
| 13% Dimethyl Isosorbide | 13% Dimethyl Isosorbide |
| 20% Transcutol | 20% Transcutol |
| 10% Hexylene glycol | 10% Hexylene glycol |
| 4% Propylene Glycol | 4% Propylene Glycol |
| 0.15% Methylparaben | 0.15% Methylparaben |
| 0.05% Propylparaben | 0.05% Propylparaben |
| 0.01% EDTA | 0.01% EDTA |
| 0.5% Carbopol Ultrez 10 | 0.3% Carbopol Ultrez 10 |
| 0.2% Penmulen TR-1 | 0.2% Penmulen TR-1 |
| 3% Isopropyl Myristate | 2% Cetyl Alcohol |
| 5% Olelyl Alcohol | 5.5% Light Mineral Oil |
| 5% White Petrolatum | 5% Oleic Acid |
| 0.02% Butylated Hydroxytoluene | 0.02% Butylated Hydroxytoluene |
| q.s. pH 6.0 25% Trolamine | q.s. pH 6.0 25% Trolamine |
| q.s. 100 Water | q.s. 100 Water |

TABLE 6
Ointment Formulations 5 and 6 of Compound 12.

| Formulation 5 (% w/w) | Formulation 6 (% w/w) |
|---|---|
| 1% Form A | 1% Form A |
| 15% PEG 400 | 10% Dimethyl Isosorbide |
| 0.02% Butylated | 0.02% Butylated Hydroxytoluene |
| Hydroxytoluene | |
| 2% Span 80 | 2% Span 80 |
| 10% White Wax | 10% White Wax |
| 71.98% White Petrolatum | 76.98% White Petrolatum |

TABLE 7
Solution Formulations 7, 8, and 9 of Compound 12.

| | | Formulation 9 |
|---|---|---|
| Formulation 7 (% w/w) | Formulation 8 (% w/w) | (% w/w) |
| 1% Form A | 1% Form A | 1% Form A |
| 15% Dimethyl Isosorbide | 15% Dimethyl Isosorbide | 99% Dimethyl |
| | | Sulfoxide |
| 25% Transcutol | 25% Transcutol | |
| 12% Hexylene glycol | 12% Hexylene glycol | |
| 5% Propylene Glycol | 5% Propylene Glycol | |
| q.s. pH 4.5 25% Trolamine | q.s. pH 6.0 25% Trolamine | |
| q.s. 100 Water | q.s. 100 Water | |

TABLE 8
Solution Formulations 10, 11, 12, 13 and 14 of Compound 12.

| | Formulation | Formulation | Formulation | Formulation | Formulation |
|---|---|---|---|---|---|
| W/W % | 10 | 11 | 12 | 13 | 14 |
| Form A | 0.1% | 0.3% | 1% | 3% | 5% |
| Sodium Bicarbonate | 0.015% | 0.046% | 0.15% | 0.46% | 0.77% |

0.1% EDTA
0.12% Sodium Phosphate, Monobasic
0.4% Methylparaben
0.02% Propylparaben
q.s. Osmolality 270, Sodium Chloride
q.s. pH 7.0 1% Sodium Hydroxide
q.s. pH 7.0 1% HCl
q.s. Water

TABLE 9
Solution Formulation 15 of Compound 12.
Formulation 15
1 ml of a solution of Compound 12
10% W/W in water, plus 0.158 mmol sodium
bicarbonate
9 ml PBS

Compound 12 can be supplied as a sterile, clear, colorless liquid solution containing 0.1%, 1.0%, and 5.0% (w/w) Active Pharmaceutical Ingredient (API) concentrations (pH 7.0). Each mL of a 1% solution contains 10 mg of the active ingredient. In addition to Compound 12, other components of a drug product solution, their functions, and their compendial grade can include propylparaben (preservative; National Formulary (NF)), methylparaben (preservative, NF), EDTA (antioxidant, United States Pharmacopeia (USP)), sodium bicarbonate (buffering agent, USP), monobasic sodium phosphate (buffering agent, USP), dibasic sodium phosphate (buffering agent, USP), and sterile water (diluent, USP). All excipients can be of compendial grade and of non-human or non-animal origin.

Formulated drug product solution can be packaged under aseptic conditions into sterile 7.0 mL High Density Polyethylene (HDPE) bottles equipped with a dropper tip that delivers an approximate per drop volume of 0.35 μL and a protective cap. The dropper bottle can have a 40 μL tip. Unpreserved study drug (no methyl or propylparabens in the formulation) can be provided in 0.5 mL unit dose Low Density Polyethylene (LDPE) containers manufactured using a blow fill seal process and stored in aluminum foil pouches.

Drug solutions can be stored refrigerated (2-8° C.). The stability of the drug at 5° C. and 25° C. can be out to 9 months or longer.

### Example 5

### In-Vitro Percutaneous Absorption of the Compound of Formula I Following Topical Application

Bioavailability following topical application in-vivo was assessed using in-vitro percutaneous absorption test methods, using procedures adapted from Skelly et al., Pharmaceutical Research 1987 4(3): 265-276, “FDA and AAPS Report of the Workshop on Principles and Practices of In-Vitro Percutaneous Penetration Studies: Relevance to Bioavailability and Bioequivalence”.

Formulations 1-9 were applied to dermatomed human skin tissue excised from a single donor in a single clinically relevant dose of 5 mg/cm², which is equivalent to a 30-35 μg dose. The thickness of the tissue ranges form 0.023 to 0.039 inches (0.584 to 0.991 mm) with a mean+/−standard deviation in thickness of 0.030+/−0.004 inches (0.773+/−0.111 mm) and a coefficient of variation of 14.4%. The tissue samples were mounted in Bronaugh flow-through diffusion cells. The cells were maintained at a constant temperature of 32° C. using recirculating water baths. The cells have a nominal diffusion area of 0.64 cm². PBS, at pH 7.4, with 0.1% sodium azide and 4% Bovine Serum Albumin was used as the receptor phase below the mounted tissue. Fresh receptor phase was continuously pumped under the tissue at a flow rate of nominally 1.0 ml/hr and collected in 6 hour intervals. The receptor phases were collected for analysis.

The tissue samples were exposed to Formulations 1-9 for 24 hours. The excess formulation residing on the strateum corneum at that timepoint was removed by tape-stripping with CuDerm D-Squame stripping discs. The tape strips were discarded. The epidermis and dermis were separated by blunt dissection. Epidermis, dermis and receptor phase were analyzed for content of Compound 12. The results are represented in Table 10.

Tissue permeation levels (the receptor phase) of Compound 12 for all formulations except for Formulation 9, which contained 99% DMSO, were below the limits of quantitation, which was 0.54 ng/ml (which is equivalent to 0.013% of the applied dose). Formulation 9, in contrast, provided 1.4% of the applied dose, permeating through all the layers of the skin tissue over the exposure period of 24 hours.

Epidermal deposition of Compound 12 over the 24 hour exposure period was very high and consistent with a large percentage of the applied dose being retained in the upper layers of the epidermis. The levels reported in Table 10 were obtained from small volume samples, which could not be re-assayed, and thus are considered underestimates of the amount of drug present in the epidermis.

Analytical data for the dermis fell within the linearity range established for Compound 12, and are quantitative. Dermal deposition of Compound 12 following a 24 hour exposure ranged from 0.66% (Formulation 6, 0.258 μg/cm²) to 4.4% (Formulation 7, 34.3 μg/cm²) of the applied dose. The concentration of Compound 12 (633.5 g/mole) in the dermis is thereby calculated as 6.74M (Formulation 6) or greater (i.e., Formulation 7 provides a concentration in the dermis of 54.1 μM) for Formulations 1 to 9 in the dermis. These concentrations are well above the in-vitro EC50 concentration for half maximal effect in inhibiting release of inflammatory cytokines by Compound 12, as shown in Example 3. These results are therefore predictive for the ability of a variety of formulations, which incorporate 1% W/W Compound 12, to provide therapeutically effective levels of in-vivo inhibition of cytokine release.

TABLE 10
Cumulative Receptor Phase and Tissue Levels of Compound 12 After 24 Hours
of Topical Exposure.

| | | Receptor Phase | | | | | | |
|---|---|---|---|---|---|---|---|---|
| | | Content at | | | | | | |
| | | 24 hours | | Epidermis | | Dermis | | |
| Formulation | | | % Dose | | % Dose | | | % Dose |
| # | | μg/cm² | Applied | μg/cm² | Applied | μg/cm² | μg/ml | Applied |
| 1 | Mean | <Limit of Quantitation | | 3.93 | 7.48 | 1.14 | 18.8 | 2.15 |
| | SD¹ | | | 2.92 | 5.50 | 0.91 | 14.9 | 1.73 |
| | % CV² | | | 74 | 74 | 80 | 80 | 80 |
| 2 | Mean | <Limit of Quantitation | | 6.03 | 11.9 | 0.750 | 12.3 | 1.49 |
| | SD | | | 2.56 | 5.1 | 0.304 | 5.0 | 0.63 |
| | % CV | | | 43 | 42 | 40 | 40 | 42 |
| 3 | Mean | <Limit of Quantitation | | 6.03 | 12.1 | 1.40 | 23.0 | 2.74 |
| | SD | | | 2.97 | 6.4 | 0.27 | 4.4 | 0.47 |
| | % CV | | | 49 | 53 | 19 | 19 | 17 |
| 4 | Mean | <Limit of Quantitation | | 7.92 | 17.0 | 0.975 | 16.0 | 2.10 |
| | SD | | | 3.41 | 7.2 | 0.350 | 5.8 | 0.75 |
| | % CV | | | 43 | 42 | 36 | 36 | 36 |
| 5 | Mean | <Limit of Quantitation | | 5.71 | 14.6 | 0.670 | 11.0 | 1.71 |
| | SD | | | 1.73 | 4.2 | 0.351 | 5.8 | 0.87 |
| | % CV | | | 30 | 29 | 52 | 52 | 51 |
| 6 | Mean | <Limit of Quantitation | | 6.47 | 16.8 | 0.258 | 4.25 | 0.657 |
| | SD | | | 1.07 | 2.7 | 0.158 | 2.6 | 0.394 |
| | % CV | | | 17 | 16 | 61 | 61 | 60 |
| 7 | Mean | <Limit of Quantitation | | 7.22 | 15.0 | 2.08 | 34.3 | 4.35 |
| | SD | | | 2.15 | 4.5 | 0.84 | 13.7 | 1.83 |
| | % CV | | | 30 | 30 | 40 | 40 | 42 |
| 8 | Mean | <Limit of Quantitation | | 8.58 | 18.0 | 1.48 | 24.3 | 3.09 |
| | SD | | | 3.53 | 7.7 | 0.99 | 16.2 | 2.07 |
| | % CV | | | 41 | 43 | 67 | 67 | 67 |
| 9 | Mean | 0.660 | 1.43 | 5.78 | 13.2 | 1.19 | 19.6 | 2.63 |
| | SD | 0.253 | 0.49 | 3.18 | 8.3 | 0.49 | 8.1 | 1.15 |
| | % CV | 38 | 34 | 55 | 63 | 41 | 41 | 44 |

¹Standard Deviation.
²Percent Coefficient of Variation.

### Example 6

### Pharmacological Activity of Compound 12 for Treatment of Keratoconjunctivitis Sicca (KCS)

Dogs were enrolled in this study if the following criteria were met: more than one year of age, a Schimer tear test (STT) of less than 10 mm wetting per minute, bilateral involvement, and at least one of the following clinical signs: blepharospasm, conjunctivial hyperemia, exposure keratopathy (irregular surface), corneal pigmentation, corneal neovascularization or ropey mucopurulent discharge, no congenital KCS, no traumatic KCS, toxic KCS, and no facial nerve paralysis. If dogs had been treated with topical CsA or tacrolimus in the previous six months, they were not enrolled.

The dogs were administered one 35 μl drop of Compound 12, 1% solution (Formulation 15, 0.35 mg/eye), in each affected eye three times daily, with approximately 4 hours (±1 hour) between the daily doses for 12 weeks. CsA will be administered for a further four weeks by administering commercially available 0.2% ointment three times a day, after the Compound 12 is discontinued at twelve weeks.

Animals were subjected to an ocular examination once during the initial visit and during five visits over sixteen weeks of the study (Weeks, 2, 4, 8, 12 and 16). The last OE was approximately four weeks after the last dose of Compound 12 and after one month of CsA treatment. The adnexa and anterior portions of both eyes were examined using an indirect opthalmoscope. The eyes were dilated with a mydriatic when applicable, to allow evaluation of the lens and fundus, including the retina. An evaluation using a modified McDonald-Shaddock scoring system was performed in conjunction with the slitlamp ocular examinations at each interval.

Tears were measured using STT strips during the initial visit and each of the five follow-up visits on Weeks 2, 4, 8, 12 and 16. One strip of STT paper was used for each eye for each interval. At each collection interval, the STT paper was folded and placed in the inferior cul de sac for sixty seconds. The length, in mm, of wetting below the notch of the paper was recorded.

Fluorescein and rose bengal staining was performed at the each of the initial and follow up examinations. Intraocular pressure measurements (IOPs) were performed using a Tono-Pet Vet® in conjunction with each of the OEs. Digital ocular images were taken before and after staining (with fluorescein and rose bengal) during each of the OEs.

Conjunctival biopsies were taken at the initial (pretreatment) visit and the Week 12 visit. The second biopsy was taken more lateral (approx. 1 mm) to the initial biopsy. Following appropriate preparation a small conjunctival biopsy was taken from the ventral fornix of each eye.

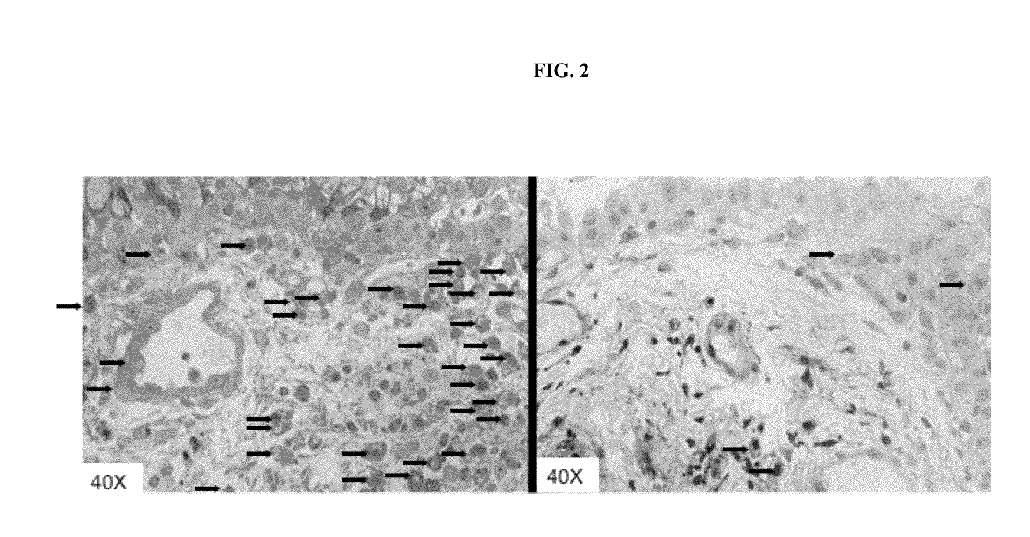

Seven dogs completed the study; for two dogs, only one eye was studied. The results are shown in Tables 11 and 12. Overall, a 3.3 mm average improvement in OD (right eye) STT and 4.5 mm in OS (left eye) STT was observed during the treatment period with Compound 12. Results for all 12 eyes show an average of 4 mm improvement. A Maximum-Minimum analysis was performed using the maximal change in STT values for each eye in each dog over weeks 1-12, as shown in Table 13. This calculation yields a total maximal change in STT for total of eyes of 72 mm, which upon division by 12 (number of KCS eyes in the analysis), yields a 6.0 mm average improvement. Other clinical signs improved in some dogs, such as a decrease in mucopurulent discharge or conjunctival erythema. Histopathological evaluation of biopsies taken before and after Compound 12 revealed an attenuation of lymphocyte accumulation. FIG. 2 illustrates this phenomenon in samples taken from dog #1. No significant additional benefit was seen from four subsequent weeks of CsA administration.

TABLE 11
Schirmer Tear Test Results (OD).

| Dog ID | Week 1 | Week 2 | Week 4 | Week 8 | Week 12 | Week 16 |
|---|---|---|---|---|---|---|
| 1 | 15 | 18 | 12 | 16 | 13 | 12 |
| 2 | 0 | 2 | 0 | 8 | 8 | 8 |
| 3 | 6 | 11 | 5 | 7 | 7 | 8 |
| 4 | 5 | 11 | 10 | 7 | 13 | 8 |
| 5 | 8 | 11 | 10 | 11 | 9 | 22** |
| 6 | 8 | 10 | 15 | 17 | 16 | 18 |
| 7 | 6 | 2 | 2 | 1 | 0 | 12 |
| Mean* | 5.5 | 7.8 | 7.0 | 8.5 | 8.8 | 11.7 |

*Dog #1 not included in mean or Maximum-Minimum analysis for OD as there is no KCS in that eye for that animal.
**Data for Dog #5 is anomalous for this day, and is not included in the mean or Maximum-Minimum analysis.

TABLE 12
Schirmer Tear Test Results (OS).

| Dog ID | Week 1 | Week 2 | Week 4 | Week 8 | Week 12 | Week 16 |
|---|---|---|---|---|---|---|
| 1 | 0 | 0 | 0 | 0 | 3 | 3 |
| 2 | 0 | 0 | 0 | 2 | 7 | 5 |
| 3 | 9 | 14 | 7 | 17 | 15 | 16 |
| 4 | 0 | 3 | 5 | 6 | 4 | 7 |
| 5 | 7 | 8 | 14 | 8 | 8 | 19 |
| 6 | 9 | 4 | 14 | 8 | 8 | 17 |
| 7 | 18 | NA | NA | 19 | 18 | 18 |
| Mean* | 4.2 | 4.8 | 6.7 | 6.5 | 8.7 | 11.0 |

*Dog #7 not included in mean or Maximum-Minimum analysis for OS as there is no KCS in that eye for that animal.

TABLE 13
Maximum-Minimum Analysis for Weeks 1-12 of
Compound 12 Administration.

| OD | OS |
|---|---|
| NA | 3 |
| 8 | 7 |
| 5 | 10 |
| 8 | 6 |
| 3 | 7 |
| 8 | 11 |
| −4 | NA |
| Total = 28 | Total = 44 |

Total OD plus Total OS: 72
Grand Total/Number of Eligible Eyes: 6.0 mm Average Improvement

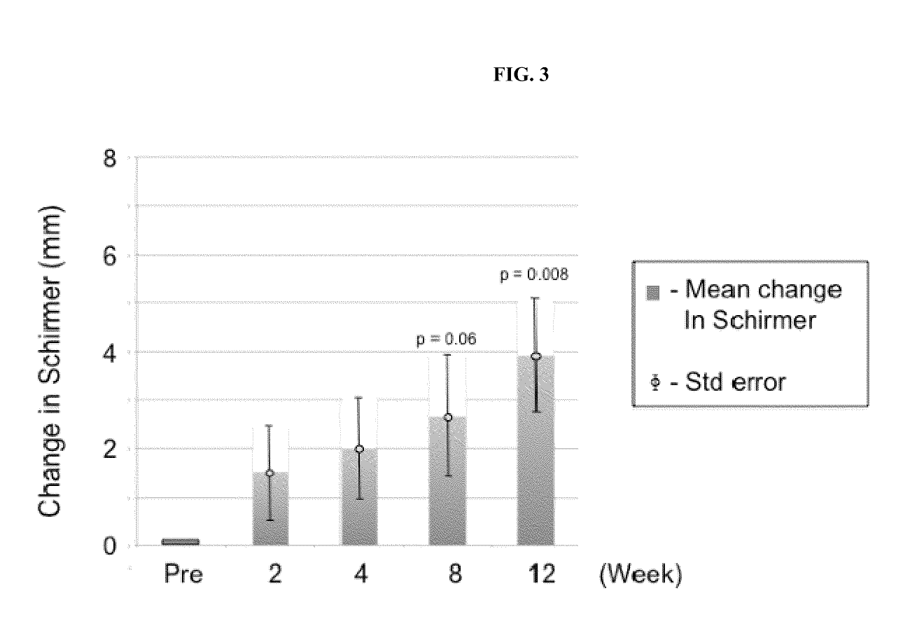

FIG. 3 illustrates the mean change in Schirmer test score at weeks 2, 4, 8, and 12. Significant improvement in Schirmer test scores over pretreatment was observed in week 12.

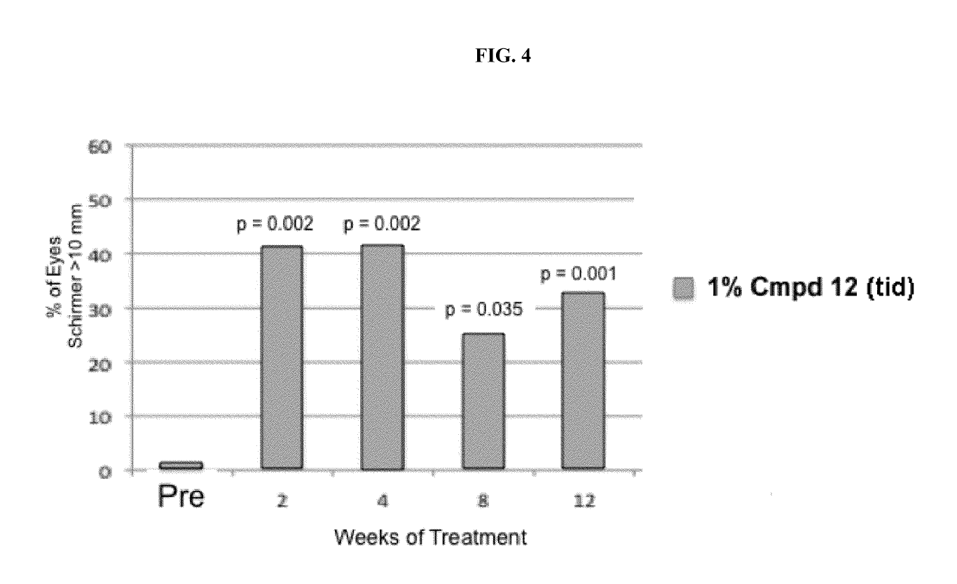

FIG. 4 illustrates the percentage of eyes with a Schirmer test score of greater than 10 mm at 2, 4, 8, and 12-weeks with 1% Compound 12 (TID). Compound 12 canine KCS study results exceeded human CsA data. The basis of restasis approval was an improvement of Schirmer test score to greater than 10 m. Restasis treatment resulted in 15% of eyes with Schirmer test score greater than 10 mm.

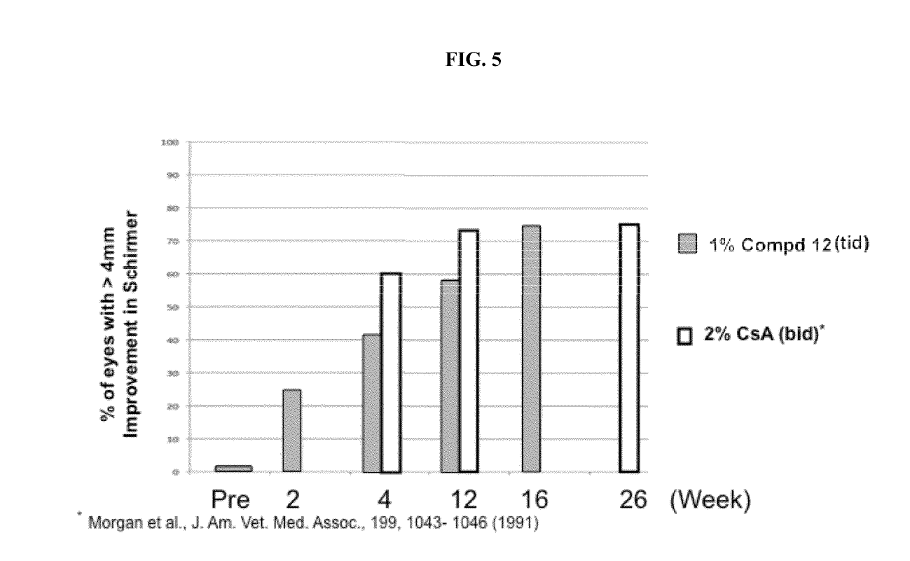

FIG. 5 illustrates the percentage of eyes with a greater than 4 mm improvement in Schirmer test score at 2, 4, 12, 16, and 26 weeks for subjects treated with 1% Compound 12 (tid) or 2% CsA (bid) (using historic CsA data; Morgan et al., J. Am. Vet. Med. Assoc., 199, 1043-1046 (1991)). Compound 12 timecourse was similar to historic CsA data.

In summary, the Canine KCS study demonstrated that administering Compound 12 resulted in rapid improvement in Schirmer test score in 2-8 weeks, improvement in histology, and rapid anti-inflammatory effect.

### Example 7

### T-Cell Proliferation Assay

This assay is an in vitro model of lymphocyte proliferation resulting from activation, induced by engagement of the T-cell receptor and LFA-1, upon interaction with antigen presenting cells (Springer, Nature 346:425 (1990)).

Microtiter plates (Nunc 96 well ELISA certified) are pre-coated overnight at 4° C. with 50 μl of 2 μg/ml of goat anti-human Fc(Caltag H10700) and 50 μl of 0.07;g/ml monoclonal antibody to CD3 (Immunotech 0178) in sterile PBS. The next day coat solutions are aspirated. Plates are then washed twice with PBS and 100 μl of 17 ng/ml 5d-ICAM-1-IgG is added for 4 hours at 37° C. Plates are washed twice with PBS prior to addition of CD4+ T cells. Lymphocytes from peripheral blood are separated from heparinized whole blood drawn from healthy donors. An alternative method is to obtain whole blood from healthy donors through leukophoresis. Blood is diluted 1:1 with saline, layered and centrifuged at 2500×g for 30 minutes on LSM (6.2 g Ficoll and 9.4 g sodium diztrizoate per 100 ml) (Organon Technica, N.J.). Monocytes are depleted using a myeloid cell depletion reagent method (Myeloclear, Cedarlane Labs, Hornby, Ontario, Canada). PBLs are resuspended in 90% heat-inactivated Fetal Bovine serum and 10% DMSO, aliquoted, and stored in liquid nitrogen. After thawing, cells are resuspended in RPMI 1640 medium (Gibco, Grand Island, N.Y.) supplemented with 10% heat-inactivated Fetal Bovine serum (Intergen, Purchase, N.Y.), 1 mM sodium pyruvate, 3 mM L-glutamine, 1 mM nonessential amino acids, 500 μg/ml penicillin, 50 μg/ml streptomycin, 50 μg/ml gentamycin (Gibco).

Purification of CD4+ T cells are obtained by negative selection method (Human CD4 Cell Recovery Column Kit # CL110-5 Accurate). 100,000 purified CD4+ T cells (90% purity) per microtiter plate well are cultured for 72 hours at 37° C. in 5% CO₂ in 100 ml of culture medium (RPMI 1640 (Gibco) supplemented with 10% heat inactivated FBS (Intergen), 0.1 mM non-essential amino acids, 1 nM Sodium Pyruvate, 100 units/ml Penicillin, 100 μg/ml Streptomycin, 50 μg/ml Gentamicin, 10 mM Hepes and 2 mM Glutamine). Inhibitors are added to the plate at the initiation of culture. Proliferative responses in these cultures are measured by addition of 1 μCi/well titrated thymidine during the last 6 hours before harvesting of cells. Incorporation of radioactive label is measured by liquid scintillation counting (Packard 96 well harvester and counter). Results are expressed in counts per minute (cpm).

### Example 8

### In Vitro Mixed Lymphocyte Culture Model

The mixed lymphocyte culture model, which is an in vitro model of transplantation (A. J. Cunningham, “Understanding Immunology, Transplantation Immunology” pages 157-159 (1978) examines the effects of various LFA-1 antagonists in both the proliferative and effector arms of the human mixed lymphocyte response.

Isolation of Cells: Mononuclear cells from peripheral blood (PBMC) are separated from heparanized whole blood drawn from healthy donors. Blood is diluted 1:1 with saline, layered, and centrifuged at 2500×g for 30 minutes on LSM (6.2 g Ficoll and 9.4 g sodium diztrizoate per 100 ml) (Organon Technica, N.J.). An alternative method is to obtain whole blood from healthy donors through leukophoresis. PBMCs are separated as above, resuspended in 90% heat inactivated Fetal Bovine serum and 10% DMSO, aliquoted and stored in liquid nitrogen. After thawing, cells are resuspended in RPMI 1640 medium (Gibco, Grand Island, N.Y.) supplemented with 10% heat-inactivated Fetal Bovine serum (Intergen, Purchase, N.Y.), 1 mM sodium pyruvate, 3 mM L-glutamine, 1 mM nonessential amino acids, 500 μg/ml penicillin, 50 μg/ml streptomycin, 50 μg/ml gentamycin (Gibco).

Mixed Lymphocyte Response (MLR): One way human mixed lymphocyte cultures are established are in 96-well flat-bottomed microtiter plates. 1.5×10⁵ responder PBMCs are co-cultured with an equal number of allogeneic irradiated (3000 rads for 3 minutes, 52 seconds stimulator PBMSc in 200 μl of complete medium. LFA-1 antagonists are added at the initiation of cultures. Cultures are incubated at 37° C. in 5% CO₂ for 6 days, then pulsed with 1 μCi/well of 3H-thymidine (6.7 Ci/mmol, NEN, Boston, Mass.) for 6 hours. Cultures are harvested on a Packard cell harvester (Packard, Canberra, Canada). [³H] TdR incorporation is measured by liquid scintillation counting. Results are expressed as counts per minute (cpm).

### Example 9

### T-Cell Adhesion Assay Using Jurkat Cells

The purpose of this study was to evaluate the anti-adhesive properties of Compound 12 on the attachment of Jurkat cells to ICAM-1 following in vitro exposure.

Stock solutions of Compound 12 and positive control were prepared in DMSO/water (1:1) and diluted into assay media and subsequent dilutions were prepared by addition of assay media to achieve the desired concentration. A reported LFA-1 antagonist was used as the positive control.

Jurkat cells were labeled with an 8 μM solution of BCECF-AM (2′,7′-bis-(2-carboxyethyl)-5-(and -6)-carboxyfluorescein) in growth media at room temperature for 15 minutes. Labeled cells were incubated in 70 μL of assay media in each well of a 96 well plate at 500,000 cells per well with 70 μL of Compound 12 or positive control in assay media at 37° C. for 30 minutes. A 100 μL aliquot of this fluorescently labeled Jurkat cell suspension was allowed to settle in the presence of Compound 12 or the positive control in wells of a 96 well plate coated with recombinant human ICAM-1 expressed as an Fc chimera at 37° C. for 1 hour. Non-adherent cells were removed by washing and centrifugation at 100 g for 1 minute. Adherent cells were determined as adherent fluorescent units on a fluorescent plate reader. The test article, Compound 12, demonstrated inhibition of Jurkat cell attachment with increasing dose. The dose response curve and IC₅₀ of Compound 12 in this assay was comparable to that of the known direct competitive LFA-1 antagonist. This demonstrates Compound 12 is an antagonist of LFA-1/ICAM-1 binding.

### Example 10

### Preclinical and Clinical Safety and Tolerability: pk (Pharmacokinetic) and Systemic and Local Distribution Results

### A. Effects in Humans

1. Phase 1 Clinical Trial Compound 12

A Phase 1 single center randomized, prospective, double masked, placebo controlled study of escalating doses of topical Compound 12 Ophthalmic Solution was conducted in 4 cohorts (0.1%, 0.3%, 1% and 5% Compound 12 dose strengths) in 28 healthy adults (7 subjects per cohort: 5 received Compound 12 Ophthalmic Solution and 2 received placebo solution). The objectives of the trial were to measure safety and tolerability, and pharmacokinetics in tear and plasma. The dosing schedule (OU; Oculus Uterque (each eye or both eyes)) was divided into 3 periods, each separated by a 72-hour wash out interval: once/day×1 day (drug one eye; placebo fellow eye), twice/day×10 days, and thrice/day×10 days, 14-day observation. Slit lamp examination of the eye, BCVA (Best Corrected Visual Acuity), STTs (Schirmer Tear Test), TBUT (Tear Break-Up Time), IOP (Intraocular pressure) were assessed at screening and the beginning and end of each period. For each cohort, masked safety data was reviewed by a Safety Committee prior to allowing dose escalation of the next cohort. A total of 2856 doses (102 drops/subject) were administered over 1148 total subject study days (41 study days/subject) in 56 eyes. All subjects in all cohorts completed the study, and no study drug doses were missed.

No deaths, discontinuations, serious or severe ocular or non-ocular AEs (adverse effects) considered related to Compound 12 Ophthalmic Solution administration occurred at any dose strength or in any dose regimen.

Blood pressure, heart rate, respiratory rate, temperature, body weight, and EKG results were within normal ranges throughout the trial.

All hematologic results and all but one serum chemistry result were within normal ranges with no observable study drug-related trends measured across study duration, dose-strength, or schedule. Total lymphocyte count, CD3, CD4, and CD8 cell counts were within normal ranges with no evidence of lymphocyte or neutrophil suppression. Urinalysis results were unremarkable throughout the trial.

Serum chemistry results were within normal range with no observable study drug-related trends measured across study duration, dose-strength, or schedule.

No serious or severe ocular or non-ocular AEs occurred during the study; there were 38 ocular (N=11 subjects) and 21 non ocular (N=11 subjects) AEs, respectively. There were no trends in the frequency of ocular AEs when analyzed by dose group or by study period. No significant safety trends were noted on BCVA, slit-lamp biomicroscopy, STT, TBUT, or IOP assessments, nor was there evidence of ocular infection, or localized immunosuppression. There was no evidence of localized ocular irritation or infection.

There were no trends in the frequency of non-ocular AEs when analyzed by dose group or by study period. No significant safety trends were noted on vital signs, EKG, laboratory studies (chemistry, liver functions, blood panels); there was no evidence of CD3, CD4, or CD8 T-cell suppression, bone marrow suppression, or clinical evidence of increased infections.

2. Pharmacokinetics in Tear and Plasma

Plasma and tear samples were obtained at baseline and during scheduled intervals in each dosing period to characterize the pharmacokinetics (PK) of Compound 12 Ophthalmic Solution following ocular administration.

a. Plasma PK Analysis

Samples for plasma Compound 12 analysis were obtained pre-dose, at 5 and 30 minutes post-dose, and at 1, 4, 8, 24 hours post-dose on Days 1, 5, 14, 18 and 27. Samples were also obtained at 48 hours post dose on Days 1, 14 and 27 and a single blood sample was collected at the follow-up visit at the end of the study. Plasma Compound 12 concentrations were determined using a validated LC/MS/MS (Liquid chromatography tandem mass spectrometry) method with a LLOQ (Lower Limit of quantitation) of 0.500 ng/mL.

b. Plasma PK Results

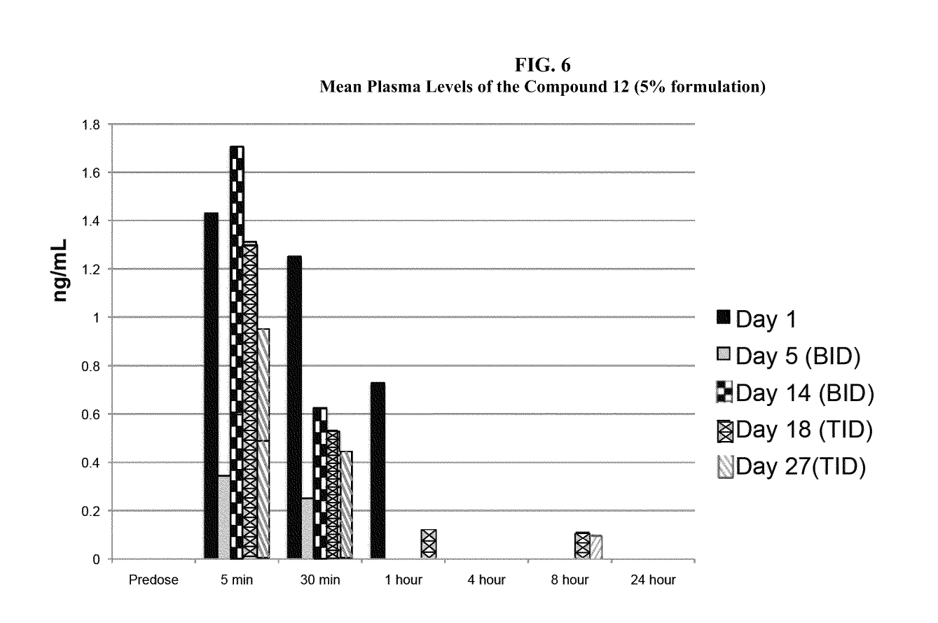

Compound 12 plasma concentrations were BLOQ (below assay lower limit of qauntitation) (<0.500 ng/mL) at all timepoints following single- and multiple-doses of 0.1% and 0.3% Compound 12 dose strengths and in 3 of 5 subjects that received the 1% Compound 12 dose strength. Measurable levels of Compound 12 were seen in the plasma of one subject dosed with 1% Compound 12 at the earliest timepoint (5 minutes post-dose) on Days 14 and 27 but were BLOQ for subsequent timepoints. Measurable levels were observed more frequently following administration of the 5% dose strength throughout the trial, although levels were quite low (<3 ng/mL) and generally were not detectable after the first hour following administration (FIG. 6). LFA-1 levels in in vitro cell assays (cell attachment and SEB IL-2 release) where IC50 values of 2 nM have been observed are approximately 0.1 nM. LFA-1 levels in blood are approximately 10 mM. The IC50 for Compound 12 inhibition of SEB stimulated IL-2 release in whole human blood is 69 nM. Compound 12 levels greater than LFA-1 levels are needed to inhibit leukocyte function. Therefore, no significant inhibition of systemic leukocytes is expected from Compound 12 ophthalmic drops.

Plasma Compound 12 half-life or exposure parameters could not be accurately assessed following administration of the Compound 12 Ophthalmic Solution at any dose strength in any study period because the plasma Compound 12 concentrations were not detectable or rapidly declined BLOQ within 1 to 4 hours of dosing.

c. Tear PK Analysis

Tear samples of Compound 12 were collected in both eyes pre-dose, at 30 minutes post-dose and at 1, 4, 8, and 24 hours post-dose on Days 1, 5, 14, 18, and 27 of the Phase 1 study using paper Schirmer tear strips. A 48-hour post-dose sample was obtained following Day 1, 14, and 27. Tear Compound 12 concentrations were determined using a validated LC/MS/MS method with a LLOQ of 0.500 ng/mL.

d. Tear PK Results

Dose related increases in tear AUC (area under the concentration-time curve) and Cₘₐₓ (maximum observed plasma concentration) values were seen on dosing day 1 and were generally maintained at the timepoints evaluated throughout the trial. BID (two times daily) and TID (three times daily) dosing produced higher Cₘₐₓ and AUC values relative to a single dose, but there were no significant differences in exposure between BID and TID dose schedules. There was clear evidence of Compound 12 exposure in the anticipated therapeutic dose range and no obvious evidence of accumulation with multiple ocular dose administration.

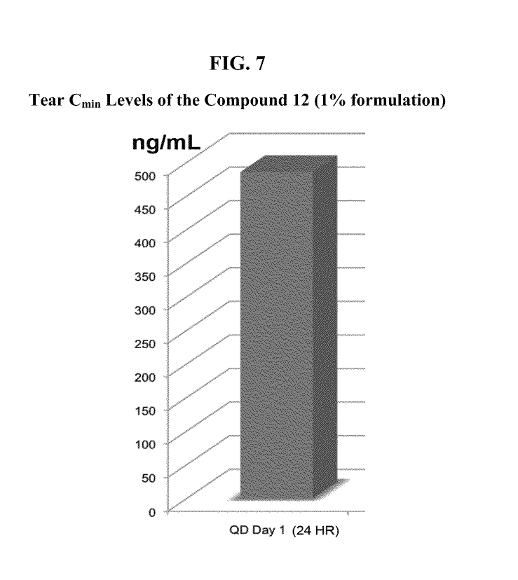

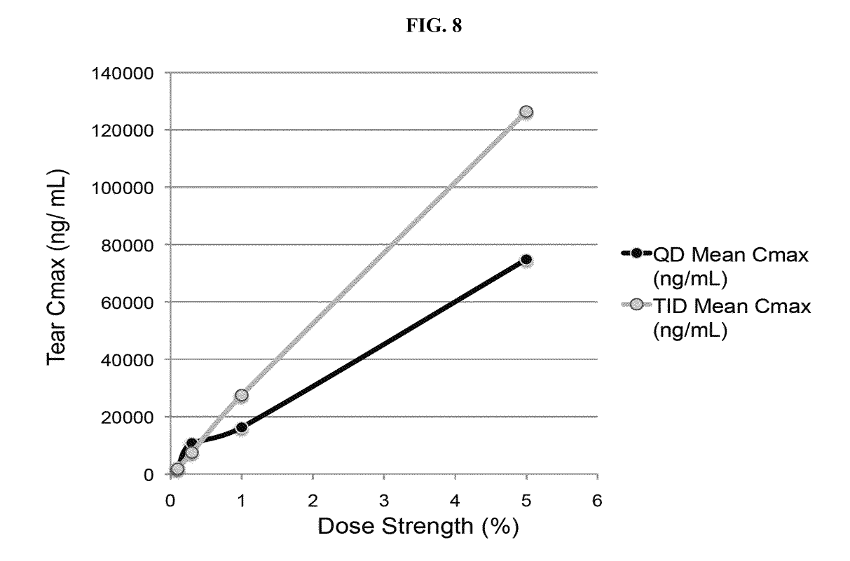

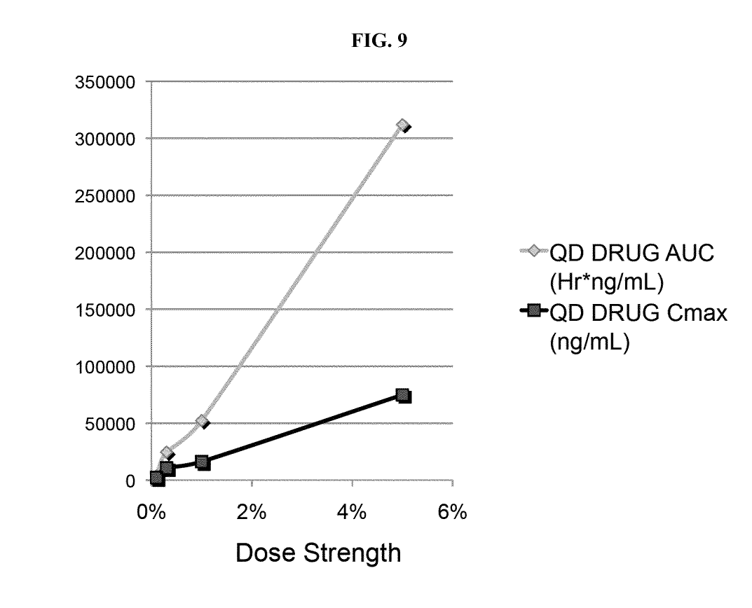

FIG. 7 illustrates 1% Compound 12 tear Cₘᵢₙ levels. FIG. 8 illustrates that dose was proportional to the Compound 12 Cₘₐₓ tear levels. FIG. 9 illustrates that dose was proportional to Compound 12 QD AUC and Cₘₐₓ in tears.

Overall, Compound 12 Ophthalmic Solution administered by topical ocular instillation to healthy adult subjects at dose strengths up to 5% TID appears safe and well-tolerated and appropriate for further investigation in subjects with ocular inflammation secondary to allergic conjunctivitis or dry eye.

### B. Nonclinical Studies Compound 12 IND-Enabling Nonclinical Program (Safety Pharmacology and Toxicology Studies)

1. Preclinical Toxicology Formulation

TABLE 14

Phosphate buffered saline
pH 7
290 mOsM/l
Compound 12 sodium salt
4 dose levels (0.1% to 3%)
EDTA
Parabens preservative
0.02% methyl parabens
0.04% propyl parabens
Multidose dropper bottle

2. Safety Pharmacology

An in vitro study to evaluate the effects of Compound 12 on hERG channel current (a surrogate for I_{Kr}, the rapidly activating, delayed rectifier cardiac potassium current) was conducted in stably transfected kidney HEK293 cells. Single doses of Compound 12 were 20 μM, 100 μM, 200 μM, and 600 μM. Compound 12 effects on the current were weak (IC₅₀ of 478 μM) indicating minimal risk of I_{Kr} channel inhibition given the low systemic exposure observed following topical ocular administration.

The cardiovascular effects of Compound 12 in conscious telemetry-instrumented dogs (beagles) when administered via IV bolus injection were assessed. No effects on electrocardiography or hemodynamic parameters were observed.

The effects of Compound 12 on the CNS when administered as a single dose via bolus IV injection were assessed in rats. Transient miosis was observed in animals given 10.0 mg/kg from 1 minute to 6 hours postdose in 2/6 animals at each time point. No effect on any other parameters was observed.

Respiratory function (tidal volume, respiration rate, and minute volume) in rats following a single IV bolus dose of Compound 12 using head-out plethysomograph chambers was assessed. No adverse changes in respiratory function or adverse effects were observed at any dose.

3. Genotoxicity Studies: Compound 12 displayed no effect in in vitro Ames chromosomal aberration assays or an in vivo rat micronucleus study.

a. In Vitro Ames Bacterial Reverse Mutation Assay

In an Ames assay, Compound 12 did not cause an increase in the mean number of revertants per plate with any of the tester strains either in the presence or absence of microsomal (S9) enzymes. Therefore, Compound 12 was judged to be not mutagenic.

b. In Vitro Chromosomal Aberration Assay in CHO cells

The ability of Compound 12 to induce chromosomal aberrations was assessed in cultured Chinese hamster ovary (CHO) cells with and without an exogenous metabolic activation following 20 hours of co-incubation. Compound 12 is considered negative for inducing structural chromosomal aberrations in CHO cells with and without metabolic activation, except at a single toxic dose without metabolic activation (3-hour treatment; 3500 μg/mL). The biological relevance of this response is equivocal due to cytotoxicity.

c. In Vivo Mouse Bone Marrow Micronucleus Assay

The ability of repeated IV administrations of Compound 12 to induce in vivo clastogenic activity and/or disruption of the mitotic apparatus, by detecting micronuclei in polychromatic erythrocytes (PCE), was assessed in CD-1® (ICR) BR mice by evaluating their bone marrow. Based on the results of this study, Compound 12 is considered negative in the mouse bone marrow micronucleus assay.

4. Acute Toxicity Studies: For single dose IV in rats, the no observable adverse effect level (NOAEL) was 10 mg/kg IV. For escalating single dose IV and 7-day repeated dose with TK (toxicokinetics) in dogs, the NOAEL was 10 mg/kg IV. For single dose ocular tolerance in rabbits, the NOAEL was 3.5 mg/eye/3× per day (10%).

5. Repeated Dose Toxicity Studies: In a 4-week IV toxicity study in dogs with 2-week recovery, the NOAEL was 10 mg/kg. In a 13-week IV toxicity study in rats with 4-week recovery, the NOAEL was 30 mg/kg. In a 13-week ocular toxicity study in rabbits with a 4-week recovery, the NOAEL was 1.05 mg/eye/3× per day (3%). In a 13-week ocular toxicity study in dogs with a 4-week recovery, the NOAEL was 1.05 mg/eye/3× per day (3%).

6. ADME Studies

The absorption, distribution, metabolism and excretion (ADME) of Compound 12 was characterized in studies conducted in rats, rabbits and dogs utilizing two routes of administration; intravenous and topical ocular administration, the clinical route of administration. An in vitro hepatocyte study was also performed.

Compound 12 levels were assessed in plasma, tear and vitreous humor samples by tandem mass spectrometry. Some in vivo studies used [¹⁴C]-Compound 12 to determine PK and the extent of absorption, distribution, and excretion of [¹⁴C]-Compound 12-derived radioactivity. Additionally, the metabolic profile and identification of metabolites of [¹⁴C]-Compound 12 were determined in plasma, urine and feces.

Single dose ocular and IV dose administration ADME studies were conducted in pigmented (Long-Evans strain) and albino (Sprague Dawley strain) rats using [¹⁴C]-Compound 12. Quantitative whole body radiography assessments were performed.

Male and female rats received a single dose of 1 mg/eye or 10 mg/kg IV [¹⁴C]-Compound 12. The main route of excretion following either ocular or IV administration was the feces, accounting for approximately 60% (ocular administration) and 95% (IV administration) of the administered radioactivity over 0 to 168 hours postdose. Urinary excretion accounted for up to 2% of the administered radioactivity. The highest tissue levels of [¹⁴C]-Compound 12 were measured in the tissues of the gastrointestinal tract with either ocular or IV dosing. With ocular administration, [¹⁴C]-Compound 12 was also measured in ocular tissues and those of excretion, indicating that the administered dose passed from the eye through the nasal turbinates, into the esophagus and was ultimately excreted through the gastrointestinal tract. These data indicate that ocular, nasal, or oral administration of Compound 12 will result in ultimate excretion through the gastrointestinal tract. The most significant proportion of drug dose administered as ocular drops, distributed locally to the periocular region, and more interestingly via nasal turbinates into the gastrointestinal tract. Drug is seen to accumulate first in the epithelium of the GI tract and pass into the liver via the portal vein, where it is eliminated from the liver and re-delivered back to the lower GI tract. Little or no drug is observed in systemic distribution. Therefore, for administration of Compound 12 via either aerosol or drops to the nose, or via oral administration may provide similar specific direct localized delivery to the epithelium of the upper GI and localized delivery to the lower GI via clearance through the liver. In both cases, little or no systemic delivery of drug may be delivered.

Following a topical ocular dose of [¹⁴C]-Compound 12 to male Sprague Dawley (albino) rats, the distribution of radioactivity into tissues was limited at the first time point (0.5 hour postdose) and was generally associated with the gastrointestinal tract, the tissues associated with metabolism, and the eye. FIG. 10 illustrates a whole body autoradiograph for a male Sprague Dawley Animal 0.5 hour after a single topical ocular administration of [¹⁴C]-Compound 12 (1 mg/eye). The highest concentrations of radioactivity were determined at this time point in esophageal contents, nasal turbinates, and small intestinal contents, with concentrations of 399000, 352000, and 349000 ng equivalents [¹⁴C]-Compound 12/g, respectively. However, it should be noted that the measurements in these tissues were above the upper limit of quantification and therefore should be interpreted with some caution. High levels of radioactivity were also determined in the esophagus and stomach contents. Radioactivity was detected in the eye at this time point, with a concentration of 18100 ng equivalents [¹⁴C]-Compound 12/g. Low levels of radioactivity were also associated with the liver (272 ng equivalents [¹⁴C]-Compound 12/g), kidney (151 ng equivalents [¹⁴C]-Compound 12/g) and uveal tract (9330 ng equivalents [¹⁴C]-Compound 12/g).

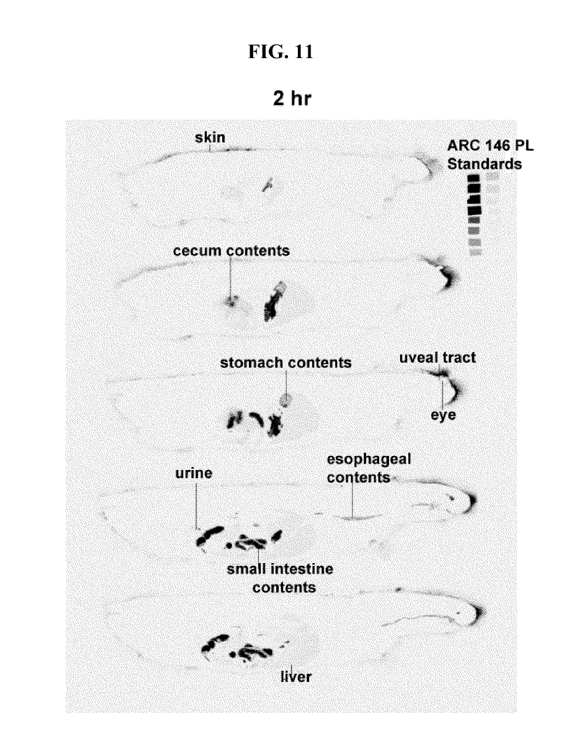

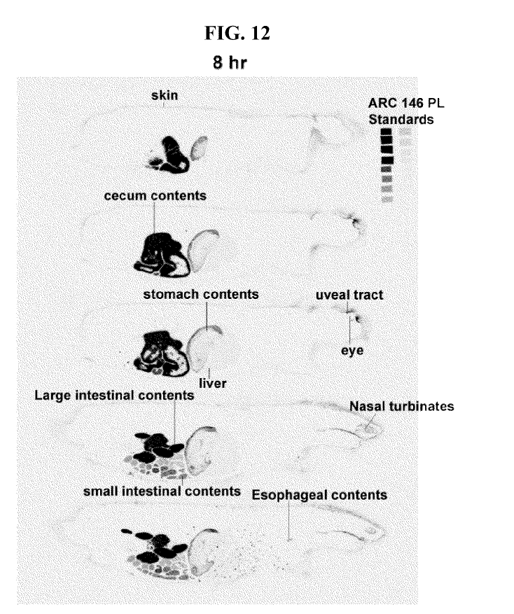

Concentrations of radioactivity in the eye and eye lens had declined considerably by 2 hours postdose; with the level in the eye lens BLQ. Radioactivity concentrations had also declined in the esophagus and esophageal contents by approximately 50- and 100-fold at 2 hours postdose. FIG. 11 illustrates a whole-body autoradiograph for a male Sprague Dawley Animal 2 hours after a single topical ocular administration of [¹⁴C]-Compound 12 (1 mg/eye). At 8 hours postdose level of radioactivity had fallen in all tissues; however, high concentrations were associated with the large intestinal contents (133000 ng equivalents [¹⁴C]-Compound 12/g) and cecum contents (57600 ng equivalents [¹⁴C]-Compound 12/g), indicating the passage of radioactivity through the gastrointestinal tract. FIG. 12 illustrates a whole-body autoradiograph for a male Sprague Dawley Animal 8 hours after a single topical ocular administration of [¹⁴C]-Compound 12 (1 mg/eye).

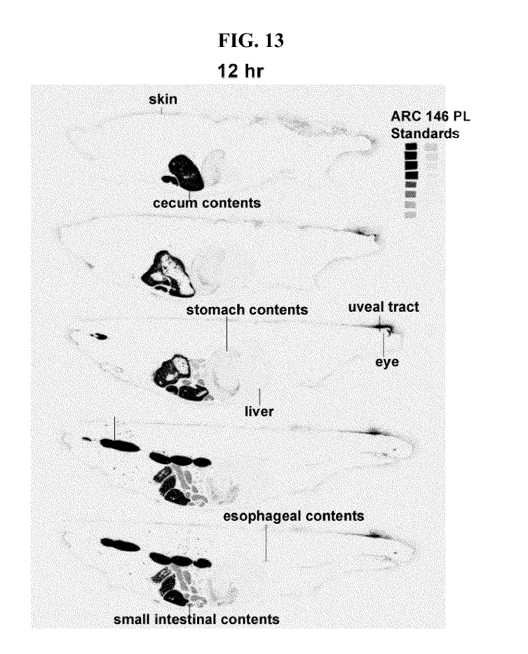

By 12 hours postdose radioactivity concentrations had decreased further, the maximal concentrations being associated with the cecum and large intestinal contents. The concentration determined in the uveal tract increased at this time point to 610 ng equivalents [¹⁴C]-Compound 12/g. FIG. 13 illustrates a whole-body autoradiograph for a male Sprague Dawley Animal 12 hours after a single topical ocular administration of [¹⁴C]-Compound 12 (1 mg/eye).

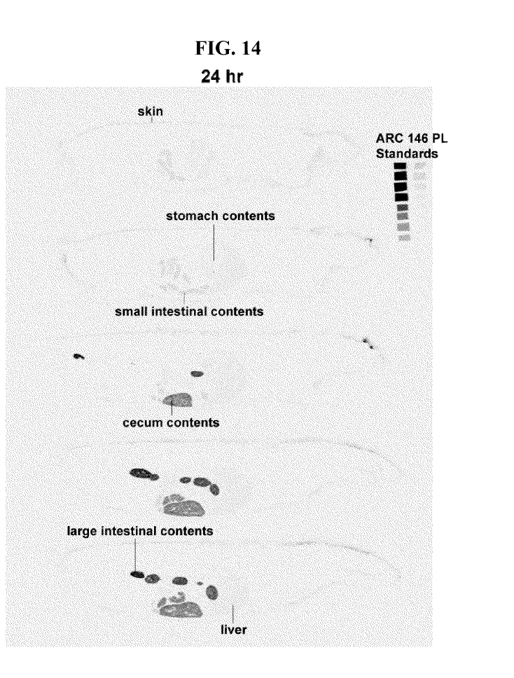

Radioactivity concentrations at 24 hours postdose were maximal in the cecum contents (5870 ng equivalents [¹⁴C]-Compound 12/g) and the large intestinal contents (18000 ng equivalents [¹⁴C]-Compound 12/g); low levels were also present in the small intestinal and stomach contents. FIG. 14 illustrates a whole-body autoradiograph for a male Sprague Dawley Animal 24 hours after a single topical ocular administration of [¹⁴C]-Compound 12 (1 mg/eye). For all other tissues, with the exception of the non-pigmented skin and the liver radioactivity was not detectable.

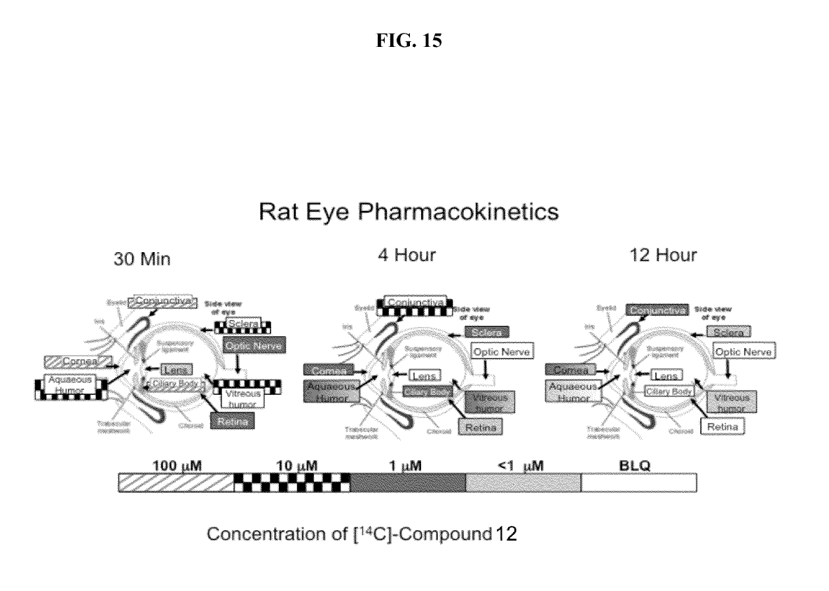

Low levels of [¹⁴C]-Compound 12 were measured in the vitreous humor at all timepoints following ocular dosing and up to 2 hrs following an IV dose (see schematic in FIG. 15 and Table 15 for ocular dosing in rats).

TABLE 15
Compound 12 Concentration, ng Equivalents [¹⁴C]-Compound
12/g tissue.

| | 0.5 hour after | 4.0 hours after |
|---|---|---|
| Physical region | administration | administration |
| Aqueous humor | 1770 | 116 |
| Conjunctiva (bulbar) | 31500 | 4480 |
| Conjunctiva (palpebral) | 26300 | 21830 |
| Cornea | 17150 | 1346 |
| Iris-ciliary body | 17550 | 500 |
| Lens | 38.8 | 9.69 |
| Optic Nerve | 796 | 0 |
| Retina and Choroid (with RPE) | 510 | 46.7 |
| Sclera | 2750 | 387 |
| Vitreous Humor | 1330 | 183 |

Tissue distribution of [¹⁴C]-Compound 12 in pigmented and albino rats was comparable and indicated that Compound 12 did not preferentially bind to melanin. There were no obvious differences seen in results from male and female rats. Furthermore, no preferential distribution of [¹⁴C]-Compound 12-derived radioactivity was seen in red blood cells and no metabolites were isolated from samples of pooled plasma, urine and fecal homogenates collected up to 168 hrs following either a topical ocular or IV dose administration of [¹⁴C]-Compound 12.

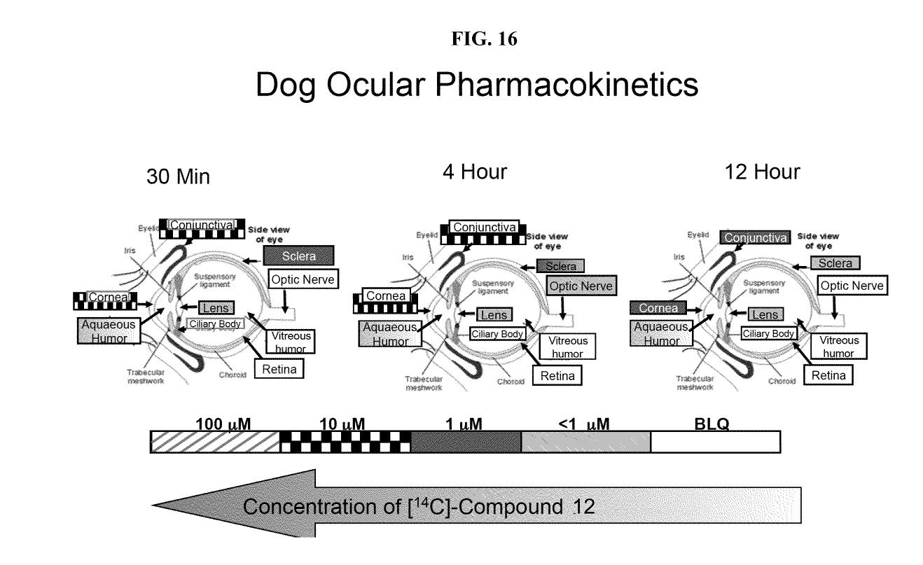

Similar single dose studies using [¹⁴C]-Compound 12 utilizing the same routes of administration were conducted in male and female dogs (3 mg/eye or 3 mg/dog) and showed comparable patterns of excretion, distribution and metabolism as rats. Following an ocular dose, the highest average [¹⁴C]-Compound 12 levels were detected in anterior ocular tissues (see FIG. 16). Lower levels were detected in posterior ocular tissues, indicating that absorption into the eye had occurred. The metabolic profile in pooled plasma, urine and fecal homogenate samples was comparable to that seen in rats, with no metabolites detected up to 168 hrs post-dose. No differences in results from male and female dogs were observed.

Compound 12 levels in conjunctiva/cornea are greater than 1 micromolar/100 nanomolar for 16 hrs (dog/rat).

a. Compound 12 Pharmacokinetics after Single and Repeated IV Administration

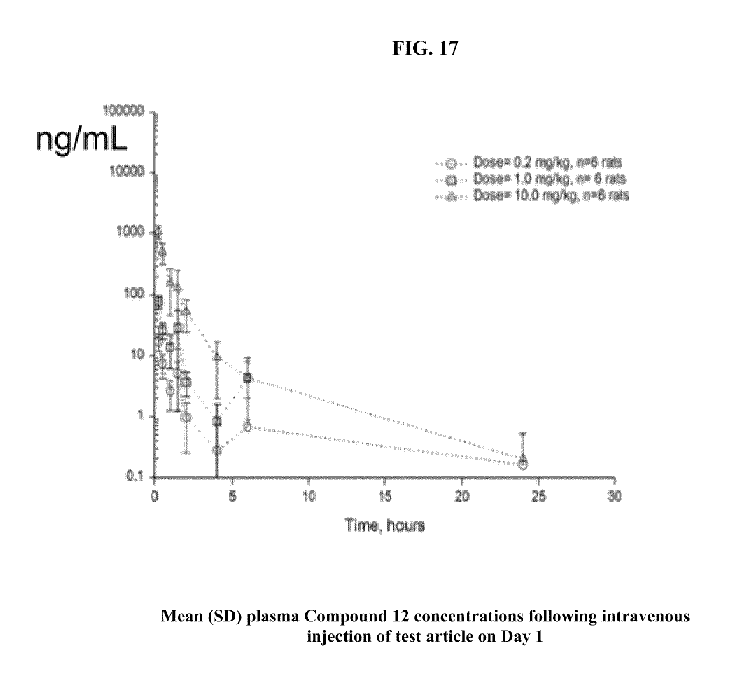

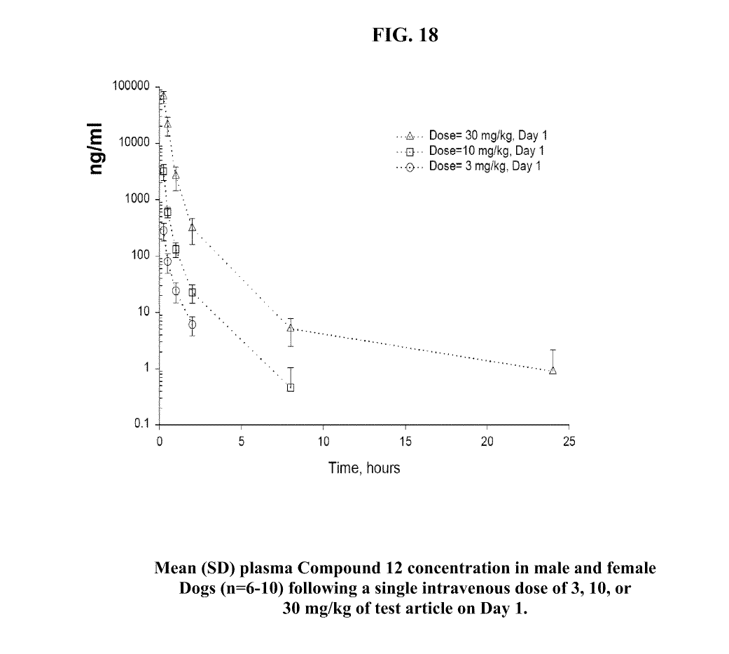

Plasma Compound 12 concentrations over time following a single IV doses in rats and dogs are shown in FIGS. 17 and 18, respectively. Plasma concentrations of Compound 12 declined in an expected, exponential manner following a single IV bolus dose in both species.

The plasma PK parameters determined using standard noncompartmental methods after a single IV administration of Compound 12 to rats at doses ranging from 0.2 to 30.0 mg/kg or to dogs after single doses up to 30 mg/kg and 7 daily doses of 3 or 10 mg/kg are shown in Table 16 (rats). PK results from both species show very high clearance of Compound 12 (liver blood flow is ˜3.3 L/hr/kg and 1.9 L/hr/kg in rats and dogs, respectively; (Davies, 1993, Pharm Res). Rat PK data indicated a high distribution volume, and moderate half-life following a single IV dose while low distribution volume and a shorter half-life drug was seen following IV administration to dogs. There was no obvious accumulation of Compound 12 in plasma after daily administration of Compound 12 for 7 days as plasma Compound 12 Cₘₐₓ and AUC₀₋ₙ values measured on Study Day 1 approximated those obtained on Study Day 7.

TABLE 16
Summary of Plasma PK Parameters Rats Following a Single IV
Bolus Dose of Compound 12³

| | CL | Vss | T_{1/2} | MRT | Cₘₐₓ | AUC₀₋ₙ |
|---|---|---|---|---|---|---|
| Dose | L/hr/kg | L/kg | hr | hr | ng/mL¹ | hr × ng/mL² |
| 10.0 mg/kg | 10.4 | 9.56 | 3.76 | 0.920 | 1056 | 728 |
| 30.0 mg/kg⁴ | — | — | — | — | 5117.3 | 2345.5 |

¹Maximum observed plasma Compound 12 concentration estimated from the mean concentration versus time profile.
²Plasma Compound 12 AUC₀₋ₙ during the dose interval estimated from the mean concentration versus time profile.
³Estimated from mean plasma Compound 12 concentration versus time profiles.
⁴From rat safety pharmacology study

In longer term repeated-dose studies, dogs and rats received daily IV bolus doses of 3, 10 or 30 mg/kg/day for 4 and 13 weeks, respectively. As was seen in the 7-day dog study, plasma Compound 12 concentrations declined in an expected, exponential manner and there was no evidence of Compound 12 accumulation in the plasma. The plasma clearance, distribution volume, and half-life of Compound 12 in dogs were dose-dependent over the dose range of 3 mg/kg to 30 mg/kg. In rats, the plasma Compound 12 exposure data suggested nonlinear disposition of Compound 12 following daily IV doses ranging from 10 to 30 mg/kg and unexpected accumulation at Week 13 (Table 17).

TABLE 17
Plasma Compound 12 Exposure Parameters in Rats Following Daily IV
Bolus Doses for 13 Weeks³

| | Dose = 3 mg/kg | | Dose = 10 mg/kg | | Dose = 30 mg/kg | |
|---|---|---|---|---|---|---|
| | | AUC₀₋ₙ | | AUC₀₋ₙ | | AUC₀₋ₙ |
| | Cₘₐₓ | hr × | Cₘₐₓ | hr × | Cₘₐₓ | hr × |
| | ng/mL¹ | ng/mL² | ng/mL¹ | ng/mL² | ng/mL¹ | ng/mL² |
| Day 1 | 305.2 | 148.3 | 1045.3 | 535.6 | 5117.3 | 2345.5 |
| Week 13 | 377.5 | 241.4 | 1691.5 | 907.1 | 16932.8 | 7471.5 |

¹Maximum observed plasma Compound 12 concentration during the dose interval.
²Plasma Compound 12 AUC₀₋ₙ during the dose interval.
³Estimated from mean plasma Compound 12 concentration versus time profile, n = 6 rats (3 males and 3 females) per timepoint.

b. Compound 12 Pharmacokinetics after Single and Repeated Ocular Administration

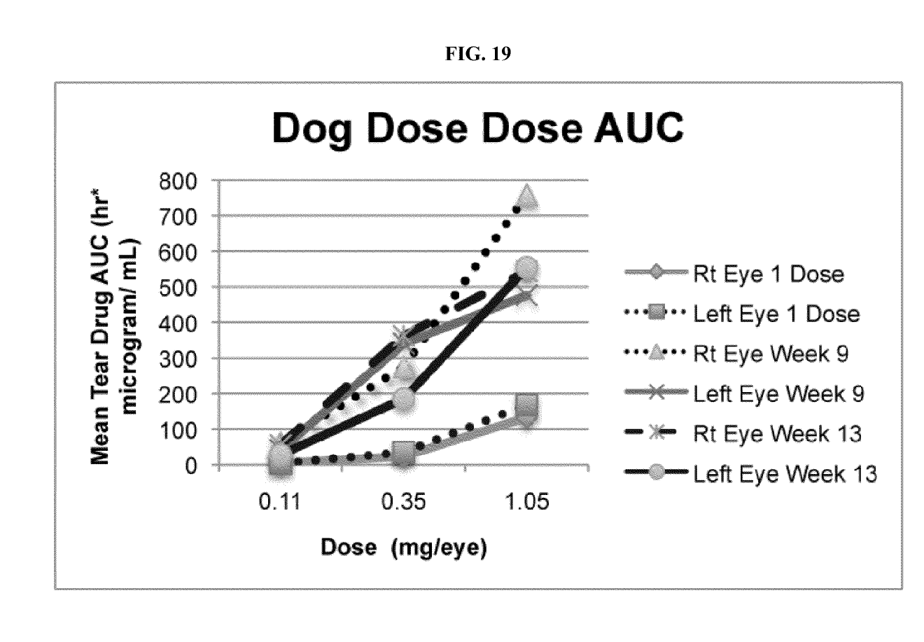

After a single topical ocular instillation of a 0.1, 1.0 or 3.0% dose strength of Compound 12Ophthalmic Solution (0.105, 0.35 and 1.05 mg/eye, respectively), mean tear Compound 12 concentrations rose in a dose-related manner achieving maximal values within 30 minutes of administration and returning to baseline by 4 hours. The tear Cₘₐₓ and AUC₀₋ₙ of Compound 12 generally increased with increasing dose. FIG. 19 illustrates that the dose of Compound 12 is proportional to PK in tears (AUC) for dogs. For example, mean tear Cₘₐₓ values were 34,014 ng/mL, 21460 ng/mL and 313,906 ng/mL in the right eyes of rabbits dosed with 0.105, 0.35 and 1.05 mg/eye, respectively. Mean tear AUCs were 18864 hr×ng/mL, 18931 hr×ng/mL and 182978 hr×ng/mL in the right eyes from the same dose groups, respectively.

Plasma Compound 12 concentrations rose after topical ocular instillation as the drug moved from the ocular application site into the plasma circulation. Dose-related amounts of Compound 12 were detected in the plasma of dogs and rabbits 30 minutes following topical ocular administration. Plasma Compound 12 concentrations rapidly declined from maximum values measured at about 0.25 hrs post-dose to baseline levels by about 4 hours, probably owing to the high Compound 12 plasma clearance as seen in the IV administration studies. Plasma Cₘₐₓ (mean±SD) values were 11.7±8.80 ng/mL, 13.1±2.12 ng/mL, and 38.9±19.7 ng/mL and AUC₀₋ₙ (mean±SD) values were 5.19±5.39 hr×ng/mL, 7.35±1.52 hr×ng/mL, and 22.9±10.1 hr×ng/mL in the 0.105, 0.35, and 1.05 mg/eye/dose groups, respectively.

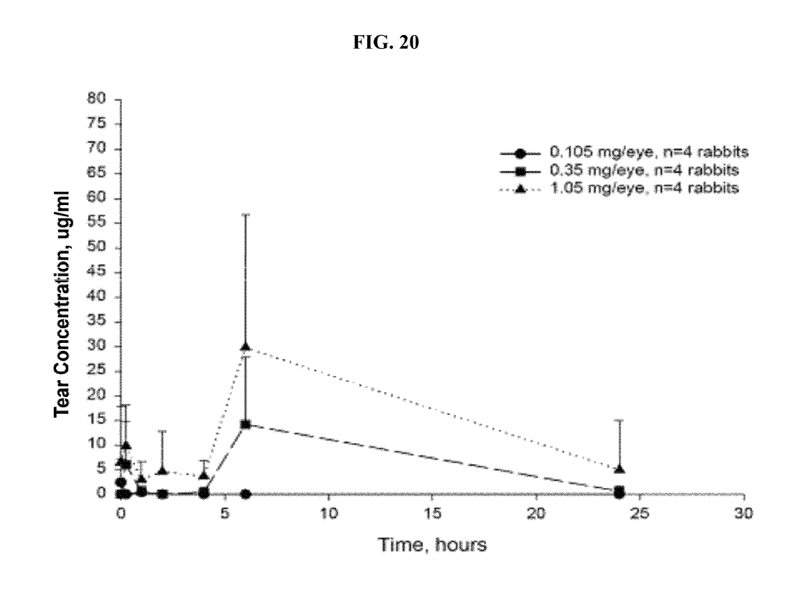

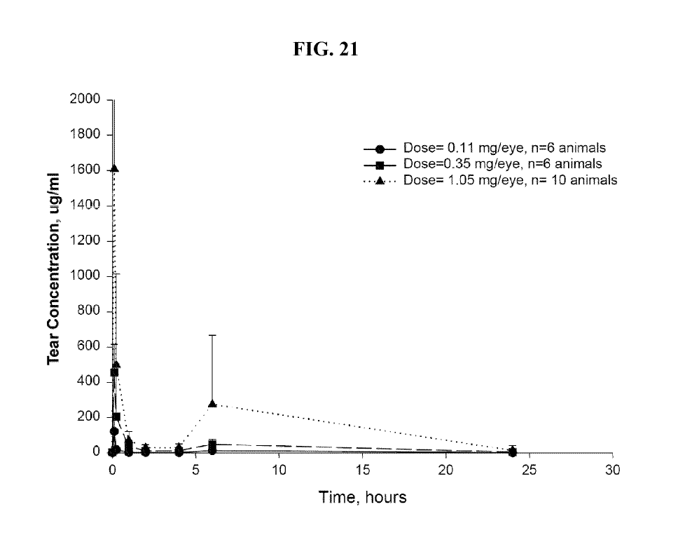

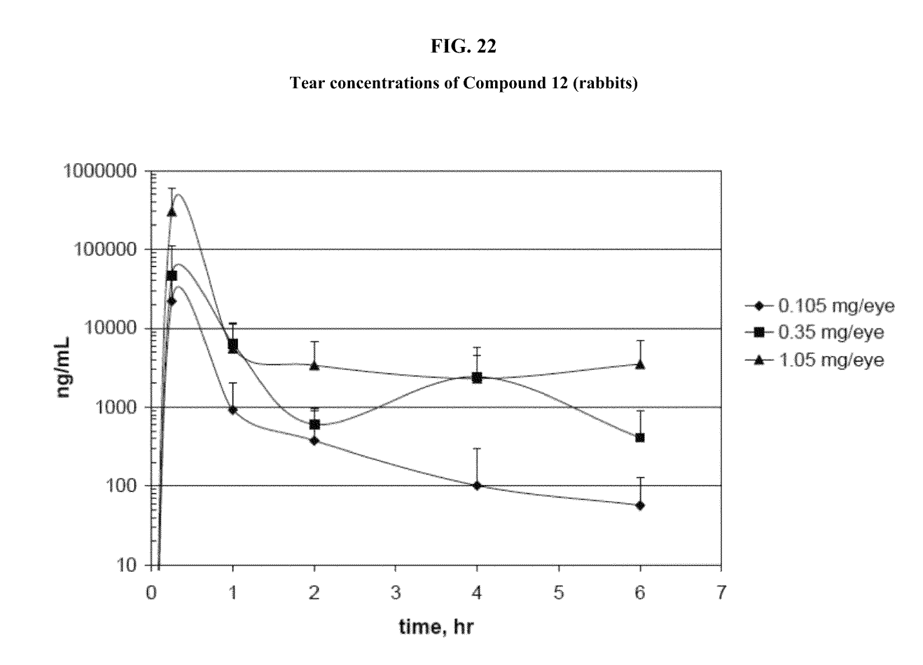

In repeated dose studies conducted in rabbits and dogs, Compound 12Ophthalmic Solution was administered TID by bilateral ocular instillation at the same doses as for single dose studies for 13 weeks. A pilot study in dogs administered 3.5 mg/eye (10% dose strength) for 3 days. The Cₘₐₓ and AUC₀₋ₙ of Compound 12 in tear samples increased expectedly with increasing dose in rabbits and dogs. The Cₘₐₓ and AUC₀₋ₙ data indicate that Compound 12 accumulated in dog tears by Week 9 during TID instillation, but thereafter continued accumulation was not noted. A similar pattern was observed in the rabbit study. Representative tear concentration over time profiles measured after 13 weeks of TID ocular dosing in rabbits and dogs are shown in FIGS. 20 and 21, respectively (left eye, TID, ˜4 hours apart). TK (toxicokinetics) analyses indicate adequate ocular Compound 12 exposure with tear levels above 1 μM (600 ng/mL) throughout the day. FIG. 22 illustrates mean Compound 12 tear concentrations in right and left eyes of rabbits following topical instillation of a single dose.

Compound 12 was not detected in the vitreous fluid in both 13-week rabbit and dog studies in samples obtained at sacrifice (terminal and recovery phase sacrifices). Variable levels of Compound 12 were seen in the vitreous fluid of dogs dosed TID for three days with 3.5 mg/eye (10%) and ranged from BLOQ to 18 ng/mL.

Nonclinical studies showed that about 6.9 to 32% of the Compound 12 ocular dose was absorbed from the ocular topical instillation site into the systemic circulation but this systemic availability estimate has been based on limited available data which includes an ocular dose that is 1/100^{th} the intravenous dose. Low systemic plasma exposure to the drug was observed in animals after ocular instillation. Importantly, the Compound 12 plasma clearance is high in these species indicating that the absorbed Compound 12 is efficiently removed from the systemic circulation, thereby assisting to minimize systemic exposure.

The PK profiles from all nonclinical species support a clinical dose topical ocular instillation regimen of up to three times per day for at least 13 weeks.

c. Pilot Ocular Tolerance of Topically Administered, Compound 12 in Dogs-PK

A pilot ocular tolerance of topically administered Compound 12 in dogs-PK was performed. Animals were dosed with 35 μL of Compound 12 TID (0, 4, 8 hrs). 1% solution was administered on days 1-14; 3% solution was administered on Days 17-21, and 10% solution was administered on Days 24-27. Compound 12 trough levels in tear/periocular tissue are greater than 1000 times the IC₅₀ for T-cell attachment/IL-2 release. Compound 12 is safe and well tolerated at up to 10% strength at 3 doses/day. Dose dependent increases in Compound 12 concentration were detected in tear (30 min-16 hours) and plasma (30 min) following ocular instillation. Vitreous concentrations of Compound 12 were greater than 1000 fold lower.

### C. Dermal

1. Compound 12 Preclinical Dermal Studies

Compound 12 displays 2% (w/w) solubility in water/glycol/transcutol solution and 10% (w/w) solubility in ethanol/glycol/transcutol solution. Solubility studies suggest an emulsion formulation. Prototypes have been developed and tested on microtomed human skin from elective surgery at 1% (w/w). The forms include gels, ointment, or lotion. Stability and compatibility has been demonstrated in all formulations. Skin transport studies performed with LC/MS/MS analysis indicate high Compound 12 levels in epidermis and dermis and low levels in the receiver. There can be greater than 10 micromolar Compound 12 in dermis, with 2-4% dose penetration, as determined using [¹⁴C]-Compound 12. Pilot rat and mini-pig studies demonstrate low systemic exposure which indicates drug penetration into vascularized levels of skin (i.e. dermis).

2. Nonclinical Dermal Program

Dermal Sensitization Study in Guinea-Pigs: Buehler Test

A Buehler test using healthy, young adult (4 to 6 weeks), randomly bred albino guinea pigs (strain Crl:(Ha)BR) is used to determine the potential of compound of Formula I to induce hypersensitivity. The diet consists of certified guinea pig diet (#5026, PMI Nutrition International LLC) ad libitum. Water is administered ad libitum. Room temperature is 18 to 26° C., relative humidity is 30 to 70%, and a 12-hour light/12-hour dark cycle is used. Animals are acclimated for at least 5 days.

Experimental design: 34 acclimated animals are placed in an irritation screening group of 4 guinea pigs, a test group of 10 guinea pigs (Group 1), a naive control group of 5 guinea pigs (Group 2), 10 positive control guinea pigs (Group 3), and 5 positive naive control guinea pigs (Group 4).

Irritation screen: Hair from the back of 4 animals is removed by clipping and four application sites per animal are selected. Each site is treated with 0.4 mL of 0.1%, 1%, or 10% w/v compound of Compound 12 and 0.4-g dose of Compound 12. Appropriate concentrations of Compound 12 are selected for induction exposure (highest to cause mild-to-moderate skin irritation) and challenge exposure (highest non-irritant dose).

Definitive phase: Prior to the test, hair is removed using electric clippers from animals in Group 1. Occlusive patch systems (Hill Top Chamber®, 25-mm diameter) are saturated with 0.4 mL solution of vehicle with a concentration of compound of Formula I as determined in the irritation screen. The occlusive patches are applied to the flanks of Group 1 guinea pigs for 6 hours. Restraints are used to maintain even pressure over the patches. The procedure is repeated on days 6-8 and 13-15 after the initial exposure. The positive control material, HCA (alpha-hexylcinnamaldehyde), 2.5% w/v in ethanol, is applied in a similar manner to the Group 3 guinea pigs. The naive control animals (Groups 2 and 4) are not treated during the induction phase.

Two weeks after the last induction patch, animals are challenged with patches saturated with a nonirritating concentration of Compound 12 applied to the dorsal anterior right quadrant, and along the dorsal anterior left quadrant with a challenge application of water. Group 2 animals (naive control) are shaved with electric clippers and treated on the dorsal anterior right quadrant with compound of formula I and along the dorsal anterior left quadrant with vehicle. HCA is administered at 5.0% and 7.0% w/v in acetone on two respective challenge sites along the right side of each animal in Group 3 in the same manner as the induction phase (0.4 mL dose volume). Group 4 animals are treated with two challenge applications of the positive control material in the same manner as Group 3.

After 6 hr, the patches are removed and the area depilated (by applying Nair®). Test sites are evaluated visually 24 and 48 hr after patch removal. Animals developing erythematous responses are considered sensitized (if irritant control animals do not respond). The number of positive reactions and the average intensity of the responses are calculated. Reactions to the challenge doses determine the sensitization. Grades of 1 or greater in the test animals to a respective material indicates evidence of sensitization, provided that grades of less than one are seen in the naive control animals to this same material. If grades of one or greater are noted in the naive control animals, then the reactions of test animals exceeding the most severe naive control reactions are considered sensitization reactions.

3. Compound 12 Pilot Rat Dermal Study

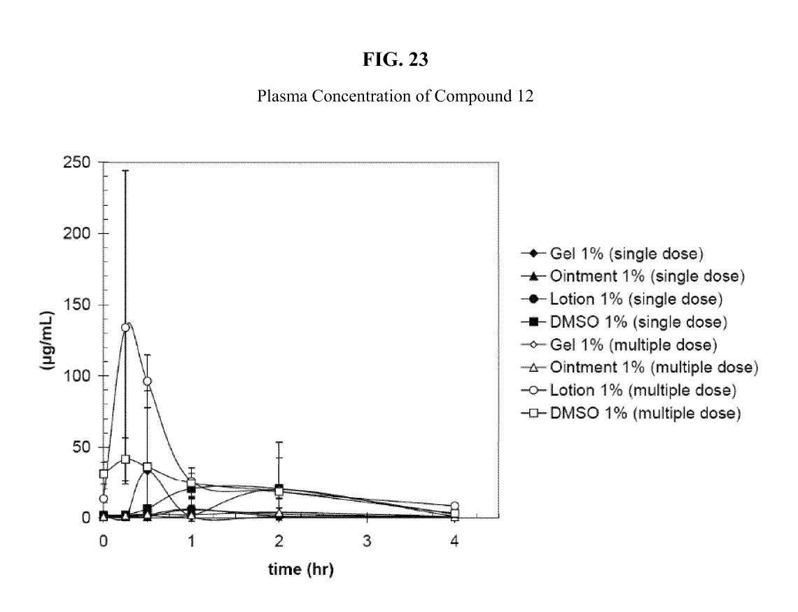

The safety and tolerability of prototypical dermal formulations (1% lotion, ointment, and gel) were assessed on rats given TID for seven consecutive days—approximately 6 cm² with 10 mg/cm². 1% DMSO was given as a high bioavailability control. FIG. 23 illustrates that Compound 12 is detectable in serum.

4. Compound 12 Pilot Mini-Pig Dermal Study

The tolerability and systemic exposure of various formulations of Compound 12 (DMSO, gel, ointment, lotion at 1%) was assessed by giving these formulations to mini-pigs as multiple dermal does TID for 7 days, approximately 50 cm² with 10 mg/cm². One pig/dose formulation was used. In-life PK analysis was completed. No toxicity was reported with any formulation. Plasma PK revealed low levels of Compound 12 in all groups but below the LLOQ of 0.5 ng/ml.

The rat and mini-pig pilot studies indicate that PK were comparable with gel and ointment and Compound 12 is safe for evaluation in humans as a gel or ointment formulation.

Prototypical 1% topical derm formulations have been developed (lotion, gel, and ointment). There is good delivery of Compound 12 to epidermis and dermis in human skin Franz cell. Pilot toxicology studies of lotion, gel, and ointment reveal the PK demonstrates good bioavailability.

### Example 11

### Crohn's Disease, Ulcerative Colitis or IBD

Subjects with Crohn's disease, ulcerative colitis or IBD will be treated with Compound 12 for up to 12 months. Drug will be supplied as a formulation suitable for oral administration (solution, pill, or capsule) containing Compound 12. A typical oral solution dosage form would include Compound 12 dissolved in PBS adjusted to pH 7. Each group of test subjects will be treated QD, BID or TID with different dose strengths of Compound 12 or placebo in formulation. Drug will be self administered by each subject by mouth. Administered dose strengths will include placebo (vehicle) 1 mg per dose, 5 mg per dose, 10 mg per dose and up to 100 mg per dose of Compound 12 in formulation.

At enrollment, patients must have a diagnosis of Crohn's disease, ulcerative colitis or IBD. Patients will be supplied with drug and required to record the administration of each drug dose in patient diaries. Treatment with Compound 12 can be used in conjunction with current anti-inflammatories (eg, salicylates) and immunosuppressants (methotrexates, steroids, antibodies).

Patients will be assessed every 2 weeks for the duration of the study. Each patient exam will include assessments of safety and tolerability. Measures of efficacy will include the Crohn's Disease Activity Index (CDAI); disease activity index or similar scale for ulcerative colitis.

Results of this trial will support regulatory claims to the treatment and maintenance of remission of Crohn's disease, ulcerative colitis and/or IBD.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A pharmaceutical formulation comprising an LFA-1 antagonist or a pharmaceutically acceptable salt or ester thereof, and an excipient suitable for oral administration, wherein the LFA-1 antagonist has a systemic clearance rate greater than about 2 mL/min/kg when administered to a subject

2. The formulation of claim 1, wherein the LFA-1 antagonist achieves a local tissue concentration of greater than about 1 μM within about 4 hours following administration to a subject.

3. The formulation of claim 2, wherein the local tissue concentration of the LFA-1 antagonist is maintained at a concentration of greater than about 10 nM for at least about 8 hours following administration to a subject.

4. The formulation of claim 1 wherein the LFA-1 antagonist is a directly competitive antagonist.

5. The formulation of claim 1, wherein the LFA-1 antagonist comprises a compound of Formula I or II and/or its pharmaceutically acceptable salts or esters, having the following structures: 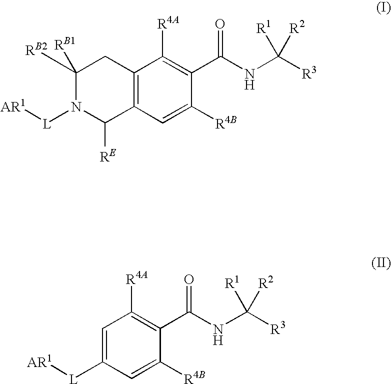
Wherein R¹ and R² are each independently hydrogen, an amino acid side chain, —(CH₂)ₘOH, —(CH₂)ₘaryl, —(CH₂)ₘheteroaryl,
wherein m is 0-6, —CH(R^{1A})(OR^{1B}), —CH(R^{1A})(NHR^{1B}), U-T-Q, or an aliphatic, alicyclic, heteroaliphatic or heteroalicyclic moiety optionally substituted with U-T-Q,
wherein U is absent, —O—, —S(O)₀₋₂—, —SO₂N(R^{1A}), —N(R^{1A})—, —N(R^{1A})C(═O)—, —N(R^{1A})C(═O)—O—, —N(R^{1A})C(═O)—N(R^{1B})—, —N(R^{1A})—SO₂—, —C(═O)—, —C(═O)—O—, —O—C(═O)—, aryl, heteroaryl, alkylaryl, alkylheteroaryl, —C(═O)—N(R^{1A})—, —OC(═O)N(R^{1A})—, —C(═N—R^{1E})—, —C(═N-R^{1E})—O—, —C(═N—R^{1E})—N(R^{1A})—, —O—C(═N—R^{1E})—N(R^{1A})—, —N(R^{1A})C(═N—R^{1E})—, —N(R^{1A})C(═N—R^{1E})—O, —N(R^{1A})C(═N—R^{1E})—N(R^{1B})—, —P(═O)(OR^{1A})—O—, or P(═O)(R^{1A})—O—;
T is absent, an aliphatic, heteroaliphatic, aryl, heteroaryl, alkylaryl or alkylheteroaryl moiety; and
Q is hydrogen, halogen, cyano, isocyanate, —OR^{1B}; —SR^{1B}; —N(R^{1B})₂, —NHC(═O)OR^{1B}, —NHC(═O)N(R^{1B})₂, —NHC(═O)R^{1B}, —NHSO₂R^{1B}, —NHSO₂N(R^{1B})₂, —NHSO₂NHC(═O)OR^{1B}, —NHC(═O)NHSO₂R^{1B}, —C(═O)NHC(═O)OR^{1B}, —C(═O)NHC(═O)R^{1B}, —C(═O)NHC(═O)N(R^{1B})₂, —C(═O)NHSO₂R^{1B}, —C(═O)NHSO₂N(R^{1B})₂, —C(═S)N(R^{1B})₂, SO₂R^{1B}, SO₂OR^{1B}, —SO₂N(R^{1B})₂, —SO₂—NHC(═O)OR^{1B}, —OC(═O)—N(R^{1B})₂, —OC(═O)R^{1B}, —OC(═O)NHC(═O)R^{1B}, —OC(═O)NHSO₂R^{1B}, —OSO₂R^{1B}, or an aliphatic heteroaliphatic, aryl or heteroaryl moiety, or wherein R¹ and R² taken together are an alicyclic or heterocyclic moiety, or together are 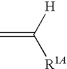
wherein each occurrence of R^{1A} and R^{1B} is independently hydrogen, an aliphatic, alicyclic, heteroaliphatic, heterocyclic, aryl, heteroaryl, alkylaryl or alkylheteroaryl moiety, —C(═O)R^{1C}, or —C(═O)NR^{1C}R^{1D}; wherein each occurrence of R^{1C} and R^{1D} is independently hydrogen, hydroxyl, or an aliphatic, heteroaliphatic, aryl, heteroaryl, alkylaryl or alkylheteroaryl moiety; and
R^{1E} is hydrogen, an aliphatic, alicyclic, heteroaliphatic, heterocyclic, aryl, heteroaryl, alkylaryl or alkylheteroaryl moiety, —CN, OR^{1C}, —NR^{1C}R^{1D} or SO₂R^{1C};
R³ is —C(═O)OR^{3A}, —C(═O)H, —CH₂OR^{3A}, —CH₂C(═O)-alkyl, —C(═O)NH(R^{3A}), —CH₂X⁰; wherein each occurrence of R^{3A} is independently hydrogen, a protecting group, an aliphatic, alicyclic, heteroaliphatic, heteroalicyclic, aryl, heteroaryl, alkylaryl, alkylheteroaryl, heteroalkylaryl heteroalkylheteroaryl moiety, or pharmaceutically acceptable salt or ester, or R^{3A}, taken together with R¹ and R², forms a heterocyclic moiety; wherein X⁰ is a halogen selected from F, Br or I;
wherein R^{4A} and R^{4B} are independently a halogen selected from F, Cl, Br or I; and R^{B1}, R^{B2} and R^{1E} are independently hydrogen or substituted or unsubstituted lower alkyl;
AR¹ is a monocyclic or polycyclic aryl, heteroaryl, alkylaryl, alkylheteroaryl, alicyclic or heterocyclic moiety; and,
L is absent or is V-W-X-Y-Z, wherein each occurrence of V, W, X, Y and Z is independently absent, C═O, NR^{L1}, —O—, —C(R^{L1})═, ═C(R^{L1})—, —C(R^{L1})(R^{L2}), C(═N—OR^{L1}), C(═NR^{L1}), —N═, S(O)₀₋₂; a substituted or unsubstituted C₁₋₆ alkenylidene or C₂₋₆ alkenylidine chain wherein up to two non-adjacent methylene units are independently optionally replaced by —C(═O)—, —CO₂—, —C(═O)C(═O)—, —C(C═O)NR^{L3}—, —OC(═O)—, —OC(═O)NR^{L3}—, —NR^{L3}NR^{L4}—, —NR^{L3}NR^{L4}C(═O)—, —NR^{L3}C(═O)—, NR^{L3}CO₂—, NR^{L3}C(═O)NR^{L4}, —S(═O)—, —SO₂—, —NR^{L3}SO₂—, —SO₂NR^{L3}, —NR^{L3}SO₂NR^{L4}, —O—, —S—, or —NR^{L3}—; wherein each occurrence of R^{L3} and R^{L4} is independently hydrogen, alkyl, heteroalkyl, aryl, heteroaryl or acyl; or an aliphatic, alicyclic, heteroaliphatic, heteroalicyclic, aryl, heteroaryl, alkylaryl or alkylheteroaryl moiety; and each occurrence of R^{L1} and R^{L2} is independently hydrogen, hydroxyl, protected hydroxyl, amino, protected amino, thio, protected thio, halogen, cyano, isocyanate, carboxy, carboxyalkyl, formyl, formyloxy, azido, nitro, ureido, thioureido, thiocyanato, alkoxy, aryloxy, mercapto, sulfonamido, benzamido, tosyl, or an aliphatic, alicyclic, heteroaliphatic, heteroalicyclic, aryl, heteroaryl, alkylaryl or alkylheteroaryl moiety, or wherein one or more occurrences of R^{L1} and R^{L2}, taken together, or taken together with one of V, W, X, Y or Z form an alicyclic or heterocyclic moiety or form an aryl or heteroaryl moiety.

6. The formulation of claim 5, wherein the LFA-1 antagonist has one of the following formulae:

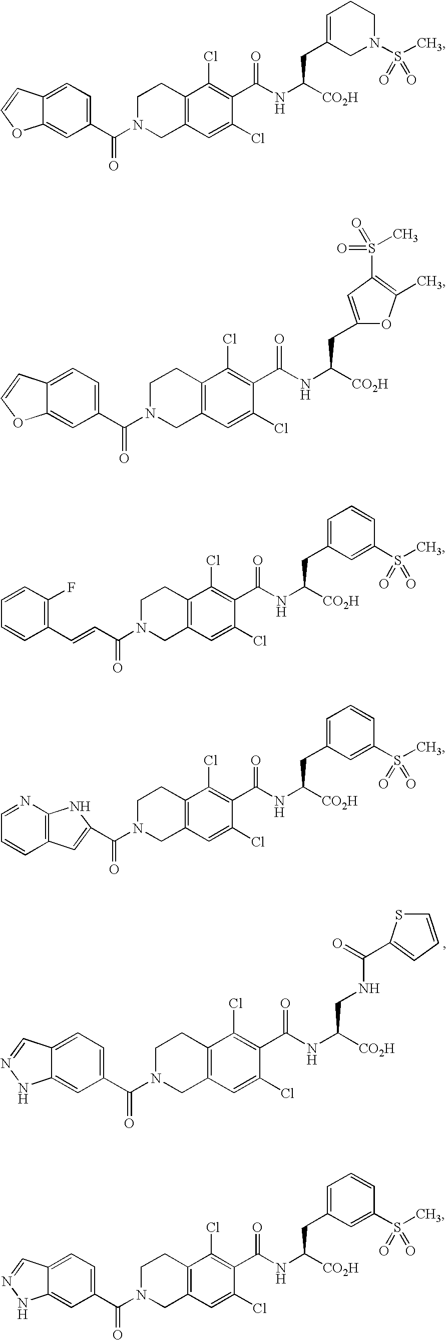

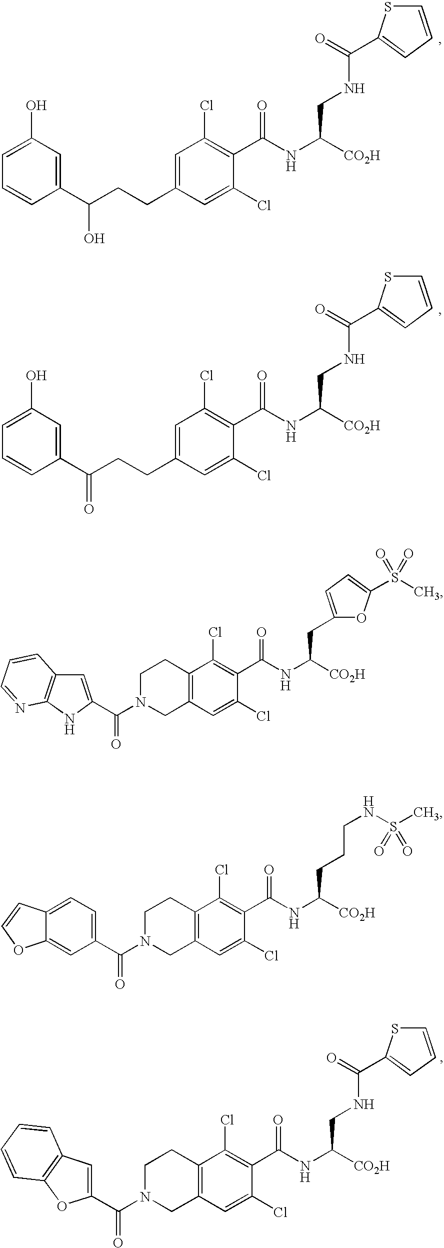

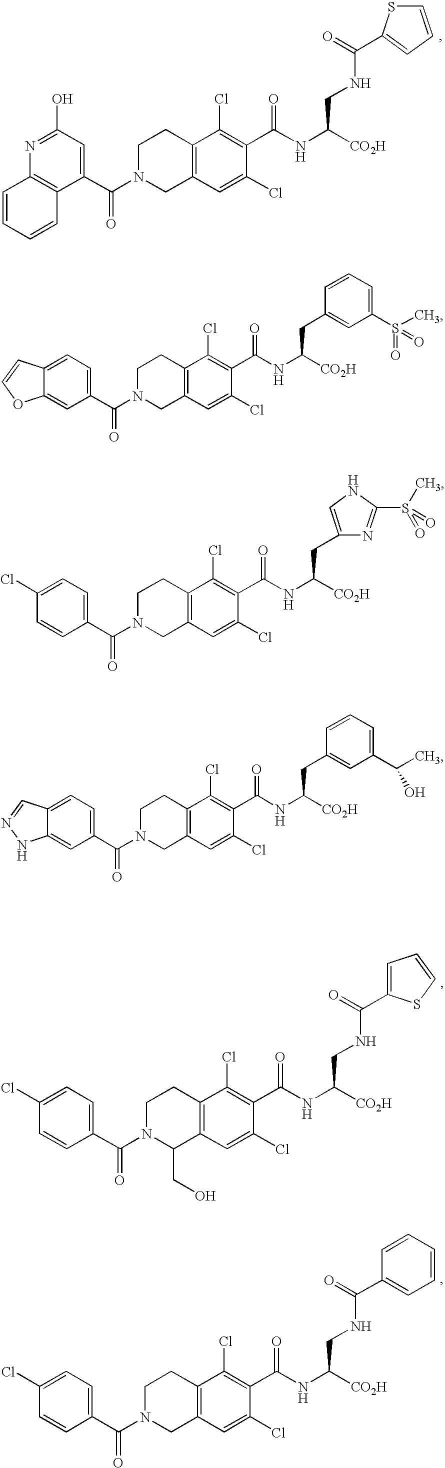

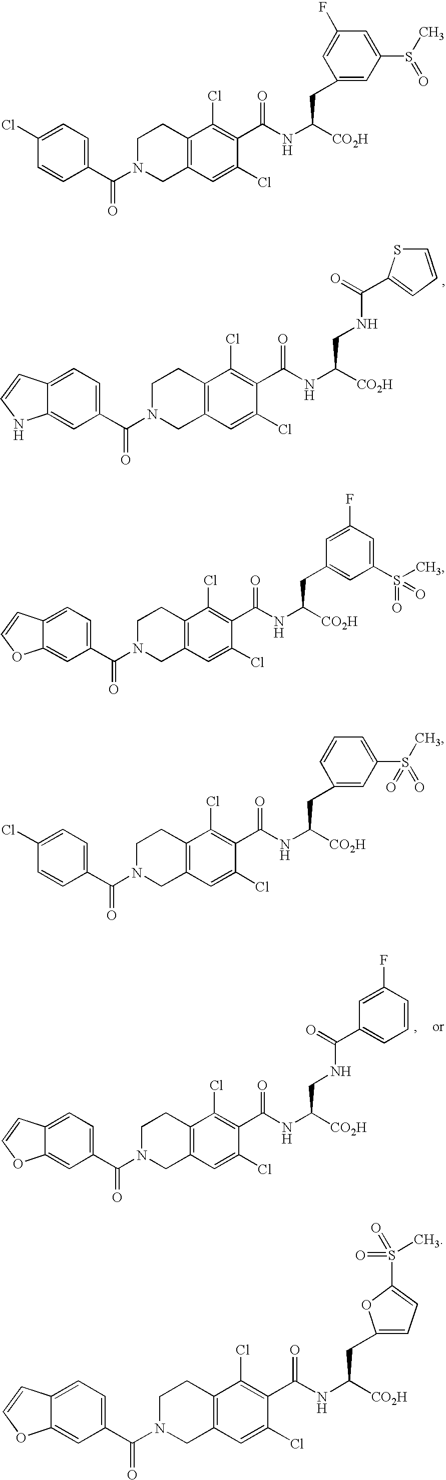

7. The formulation of claim 5 or 6 wherein the LFA-1 antagonist is a sodium, potassium, lithium, magnesium, zinc, or calcium salt.

8. The formulation of claim 1, wherein the LFA-1 antagonist inhibits T-cell attachment to ICAM-1 by about 50% or more at a concentration of about 100 nM.

9. The formulation of claim 1, wherein the formulation is in the form of a tablet, capsule, suspension, powder, crystalline forms, suppository, microparticle, or nanoparticle.

10. The formulation of claim 1, wherein the excipient is water, buffered aqueous solution, surfactant, volatile liquid, starch, polyol, granulating agent, microcrystalline cellulose, diluent, lubricant, acid, base, salt, emulsion, oil, wetting agent, chelating agent, antioxidant, sterile solution, complexing agent or disintegrating agent.

11. The formulation of claim 9, wherein the surfactant is oleic acid, cetylpyridinium chloride, soya lecithin, polyoxyethylene sorbitan monolaurate, polyoxyethylene sorbitan monostearate, polyoxyethylene sorbitan monooleate, polyoxyethylene stearyl ether, polyoxyethylene oleyl ether, polyoxyethylene-polyoxypropylene-ethylenediamine block copolymer, polyoxypropylene-polyoxyethylene block copolymer or castor oil ethoxylate.

12. The formulation of claim 1, further comprising a topical penetration enhancer.

13. The formulation of claim 11, wherein the topical penetration enhancer is a sulfoxide, ether, surfactant, alcohol, fatty acid, fatty acid ester, polyol, amide, terpene, alkanone or organic acid.

14. The formulation of claim 1, further comprising at least one additional therapeutic agent which is a 5-aminosalicylates (5-ASA) compound, corticosteroid, antibiotic, calcineurin inhibitor, or immunomodulator.

15. The formulation of claim 14, wherein the 5-ASA compound is sulfasalazine, osalazine, or mesalamine.

16. The formulation of claim 14, wherein the corticosteroid is prednisone or budesonide.

17. The formulation of claim 14, wherein the antibiotic is metronidazole or ciprofloxacin.

18. The formulation of claim 14, wherein the immunomodulator is 6-mercaptopurine, azathioprine, methotrexate, infliximab, or adalimumab.

19. The formulation of claim 14, wherein the calcineurin inhibitor is cyclosporine, tacrolimus, pimecrolimus, or sirolimus.

20. The formulation of claim 6 wherein the LFA-1 antagonist is a compound having the following formula:

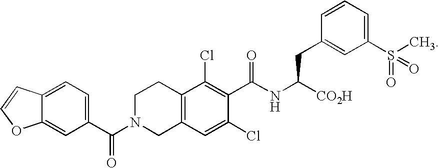

21. The formulation of claim 20 wherein the LFA-1 antagonist is any of crystalline Forms A, B, C, D, or E, the amorphous form or a combination thereof.

22. The formulation of claim 21 wherein the LFA-1 antagonist is Form A of the compound of claim 20.

23. A method for treatment of an inflammatory or immune related disorder of one or more tissues of the gastrointestinal system in a subject comprising administering to said subject in need thereof, a formulation comprising an LFA-1 antagonist or a pharmaceutically acceptable salt or ester thereof, and a pharmaceutically acceptable excipient, wherein the LFA-1 antagonist has a systemic clearance rate greater than about 2 mL/min/kg when administered to a subject.

24. The method of claim 23, wherein following administration, the LFA-1 antagonist is present in a therapeutically effective concentration within about 1 mm of an epithelial surface to which the formulation is delivered and is present in blood plasma below a therapeutically effective level, within about 4 hours following administration.

25. The method of claim 23, wherein the LFA-1 antagonist has a local tissue concentration of greater than about 10 nM within about 4 hours following administration to the subject.

26. The method of claim 23 wherein the LFA-1 antagonist has a local tissue concentration of greater than about 1 μM and a systemic concentration as measured in plasma of less than about 100 nM, within about 4 hours following administration to the subject.

27. The method of claim 25, wherein the LFA-1 antagonist maintains the local tissue concentration of greater than about 10 nM for at least about 8 hours following administration to a subject.

28. The method of claim 25, wherein the local tissue concentration of the LFA-1 antagonist is within about 1 mm of an epithelial surface to which the formulation is applied.

29. The method of claim 23, wherein the LFA-1 antagonist is a directly competitive antagonist.

30. The method of claim 23, wherein the LFA-1 antagonist is a compound of Formula (I) or (II) and its pharmaceutically acceptable salts or esters, having the following structures: 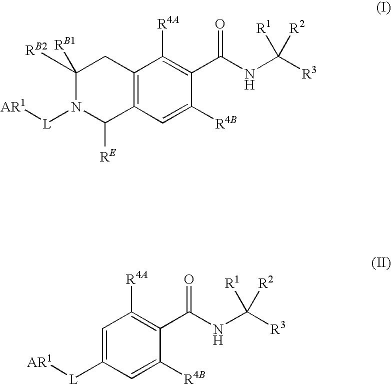
wherein R¹ and R² are each independently hydrogen, an amino acid side chain, —(CH₂)ₘOH, —(CH₂)ₘaryl, —(CH₂)ₘheteroaryl,
wherein m is 0-6, —CH(R^{1A})(OR^{1B}), —CH(R^{1A})(NHR^{1B}), U-T-Q, or an aliphatic, alicyclic, heteroaliphatic or heteroalicyclic moiety optionally substituted with U-T-Q,
wherein U is absent, —O—, —S(O)₀₋₂—, —SO₂N(R^{1A}), —N(R^{1A})—, —N(R^{1A})C(═O)—, —N(R^{1A})C(═O)—O—, —N(R^{1A})C(═O)—N(R^{1B})—, —N(R^{1A})—SO₂—, —C(═O)—, —C(═O)—O—, —O—C(═O)—, aryl, heteroaryl, alkylaryl, alkylheteroaryl, —C(═O)—N(R^{1A})—, —OC(═O)N(R^{1A})—, —C(═N—R^{1E})—, —C(═N—R^{1E})—O—, —C(═N—R^{1E})—N(R^{1A})—, —O—C(═N—R^{1E})—N(R^{1A})—, —N(R^{1A})C(═N—R^{1E})—, —N(R^{1A})C(═N—R^{1E})—O—, —N(R^{1A})C(═N—R^{1E})—N(R^{1B})—, —P(═O)(OR^{1A})—O—, or —P(═O)(R^{1A})—O—;
T is absent, an aliphatic, heteroaliphatic, aryl, heteroaryl, alkylaryl or alkylheteroaryl moiety; and
Q is hydrogen, halogen, cyano, isocyanate, —OR^{1B}; —SR^{1B}; —N(R^{1B})₂, —NHC(═O)OR^{1B}, —NHC(═O)N(R^{1B})₂, —NHC(═O)R^{1B}, —NHSO₂R^{1B}, NHSO₂N(R^{1B})₂, —NHSO₂NHC(═O)OR^{1B}, —NHC(═O)NHSO₂R^{1B}—C(═O)NHC(═O)OR^{1B}, —C(═O)NHC(═O)R^{1B}, —C(═O)NHC(═O)N(R^{1B})₂, —C(═O)NHSO₂R^{1B}, —C(═O)NHSO₂N(R^{1B})₂, —C(═S)N(R^{1B})₂, —SO₂R^{1B}, —SO₂OR^{1B}, —SO₂N(R^{1B})₂, —SO₂—NHC(═O)OR^{1B}, —OC(═O)—N(R^{1B})₂, —OC(═O)R^{1B}, —OC(═O)NHC(═O)R^{1B}, —OC(═O)NHSO₂R^{1B}, —OSO₂R^{1B}, or an aliphatic heteroaliphatic, aryl or heteroaryl moiety, or wherein R¹ and R² taken together are an alicyclic or heterocyclic moiety, or together are 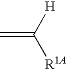
wherein each occurrence of R^{1A} and R^{1B} is independently hydrogen, an aliphatic, alicyclic, heteroaliphatic, heterocyclic, aryl, heteroaryl, alkylaryl or alkylheteroaryl moiety, —C(═O)R^{1C}, or —C(═O)NR^{1C}R^{1D}; wherein each occurrence of R^{1C} and R^{1D} is independently hydrogen, hydroxyl, or an aliphatic, heteroaliphatic, aryl, heteroaryl, alkylaryl or alkylheteroaryl moiety; and
R^{1E} is hydrogen, an aliphatic, alicyclic, heteroaliphatic, heterocyclic, aryl, heteroaryl, alkylaryl or alkylheteroaryl moiety, —CN, —OR^{1C}, NR^{1C}R^{1D} or —SO₂R^{1C};
R³ is —C(═O)OR^{3A}, —C(═O)H, —CH₂OR^{3A}, —CH₂C(═O)-alkyl, —C(═O)NH(R^{3A}), —CH₂X⁰; wherein each occurrence of R^{3A} is independently hydrogen, a protecting group, an aliphatic, alicyclic, heteroaliphatic, heteroalicyclic, aryl, heteroaryl, alkylaryl, alkylheteroaryl, heteroalkylaryl heteroalkylheteroaryl moiety, or pharmaceutically acceptable salt or ester, or R^{3A}, taken together with R¹ and R², forms a heterocyclic moiety; wherein X⁰ is a halogen selected from F, Br or I;
wherein R^{4A} and R^{4B} are independently a halogen selected from F, Cl, Br or I; and R^{B1}, R^{B2} and R^{E} are independently hydrogen or substituted or unsubstituted lower alkyl;
AR¹ is a monocyclic or polycyclic aryl, heteroaryl, alkylaryl, alkylheteroaryl, alicyclic or heterocyclic moiety; and,
L is absent or is V-W-X-Y-Z, wherein each occurrence of V, W, X, Y and Z is independently absent, C═O, NR^{L1}, —O—, —C(R^{L1})═, ═C(R^{L1}), —C(R^{L1})(R^{L2}), C(═N—OR^{L1}), C(═NR^{L1}), —N═, S(O)₀₋₂; a substituted or unsubstituted C₁₋₆ alkenylidene or C₂₋₆ alkenylidine chain wherein up to two non-adjacent methylene units are independently optionally replaced by —C(═O)—, —CO₂—, —C(═O)C(═O)—, —C(C═O)NR^{L3}—, —OC(═O)—, —OC(═O)NR^{L3}—, —NR^{L3}NR^{L4}—, —NR^{L3}NR^{L4}C(═O)—, —NR^{L3}C(═O)—, NR^{L3}CO₂—, NR^{L3}C(═O)NR^{L4}, —S(═O)—, —SO₂—, —NR^{L3}SO₂—, —SO₂NR^{L3}, —NR^{L3}SO₂NR^{L4}, —O—, —S—, or —NR^{L3}—; wherein each occurrence of R^{L3} and R^{L4} is independently hydrogen, alkyl, heteroalkyl, aryl, heteroaryl or acyl; or an aliphatic, alicyclic, heteroaliphatic, heteroalicyclic, aryl, heteroaryl, alkylaryl or alkylheteroaryl moiety; and each occurrence of R^{L1} and R^{L2} is independently hydrogen, hydroxyl, protected hydroxyl, amino, protected amino, thio, protected thio, halogen, cyano, isocyanate, carboxy, carboxyalkyl, formyl, formyloxy, azido, nitro, ureido, thioureido, thiocyanato, alkoxy, aryloxy, mercapto, sulfonamido, benzamido, tosyl, or an aliphatic, alicyclic, heteroaliphatic, heteroalicyclic, aryl, heteroaryl, alkylaryl or alkylheteroaryl moiety, or wherein one or more occurrences of R^{L1} and R^{L2}, taken together, or taken together with one of V, W, X, Y or Z form an alicyclic or heterocyclic moiety or form an aryl or heteroaryl moiety.

31. The method of claim 23, wherein the LFA-1 antagonist has one of the following formulae:

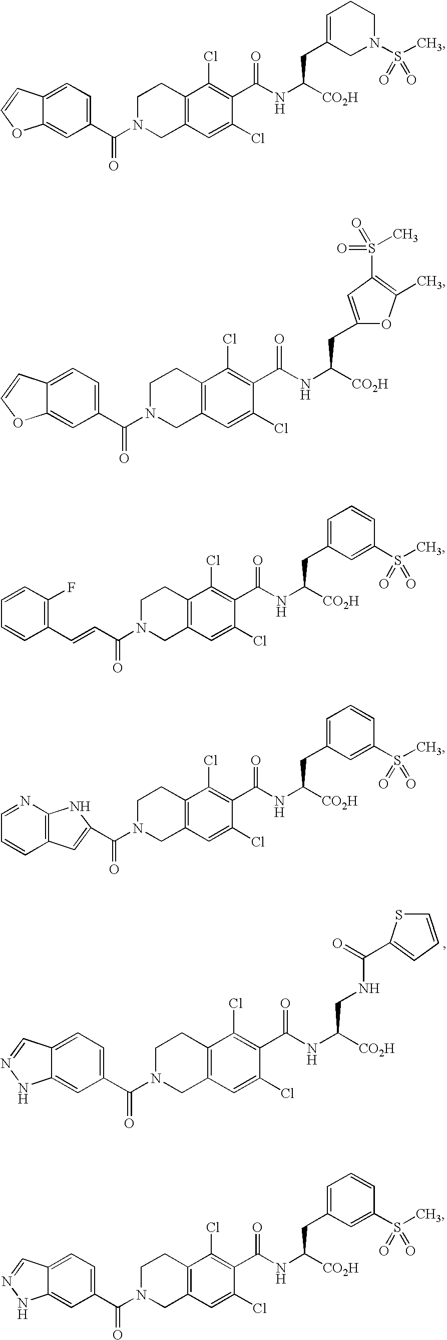

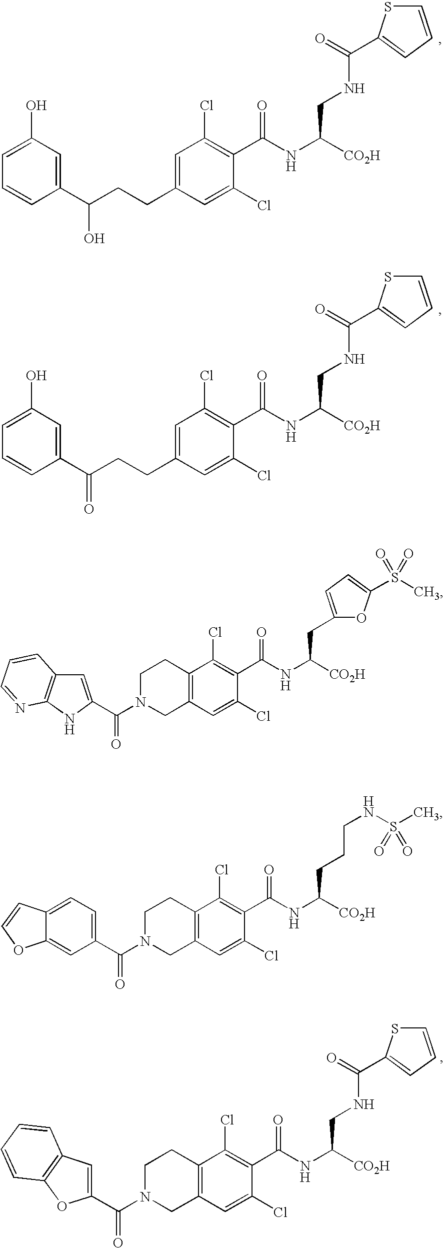

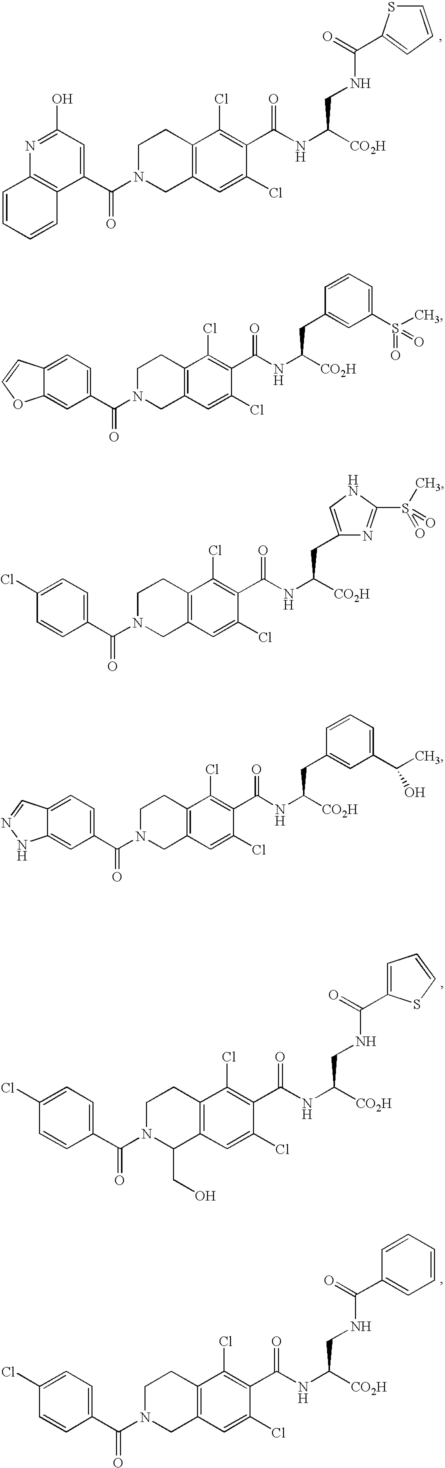

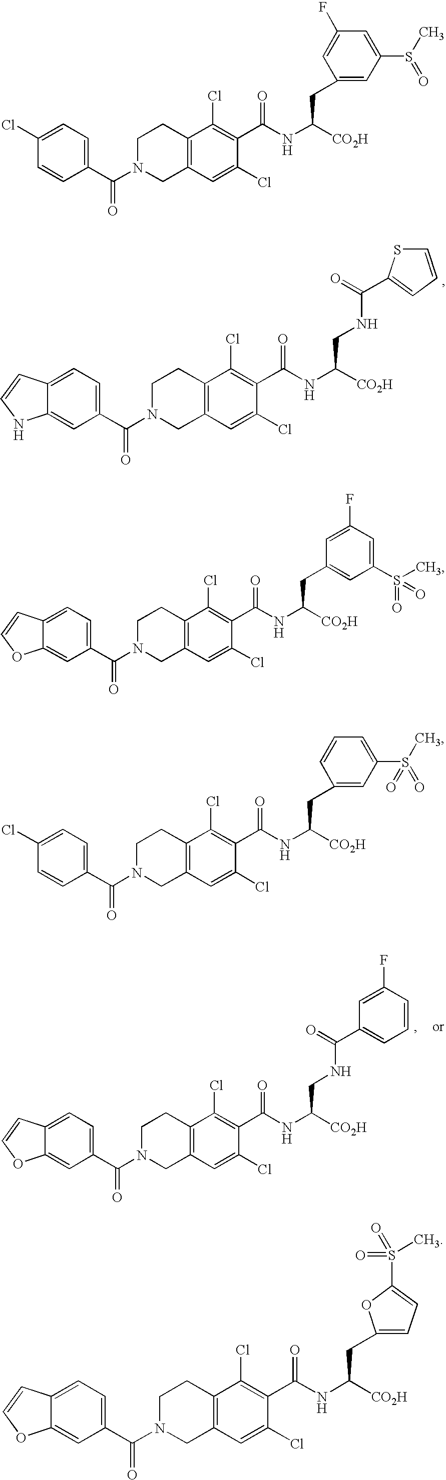

32. The method of claim 31 wherein the LFA-1 antagonist is a compound having the following formula:

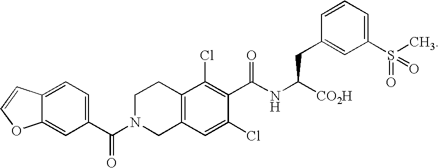

33. The method of claim 32 wherein the LFA-1 antagonist is any of crystalline Forms A, B, C, D, or E, the amorphous form or a combination thereof, of the compound of claim 32.

34. The method of claim 33, wherein the LFA-1 antagonist is Form A of the compound of claim 32.

35. The method of claim 23, wherein the LFA-1 antagonist inhibits T-cell attachment to ICAM-1 by about 50% or more at a concentration of about 100 nM.

36. The method of claim 23, wherein the formulation is a tablet, capsule, suspension, powder, crystalline forms, suppository, microparticle, or nanoparticle.

37. The method of claim 23, wherein the formulation is applied to anal mucosa.

38. The method of claim 23, wherein the formulation is orally administered.

39. The method of claim 23, further comprising administering to the subject an additional therapeutic agent.

40. The method of claim 39, wherein administering the additional therapeutic agent is concurrent with, prior to, or subsequent to administering the LFA-1 antagonist therapeutic agent or a pharmaceutically acceptable salt or ester thereof.

41. The method of claim 39, wherein the additional therapeutic agent is an antioxidant, antiinflammatory agent, antimicrobial agent, antiangiogenic agent, or anti-apoptotic agent.

42. The method of claim 41, wherein the additional therapeutic agent is a 5-aminosalicylates (5-ASA) compound, corticosteroid, antibiotic, calcineurin inhibitor, or immunomodulator.

43. The method of claim 41, wherein the 5-ASA compound is sulfasalazine, osalazine, or mesalamine.

44. The method of claim 42, wherein the corticosteroid is prednisone or budesonide.

45. The method of claim 42, wherein the antibiotic is metronidazole or ciprofloxacin.

46. The method of claim 42, wherein the immunomodulator is 6-mercaptopurine, azathioprine, methotrexate, infliximab, or adalimumab.

47. The method of claim 42, wherein the calcineurin inhibitor is cyclosporine, tacrolimus, pimecrolimus, or sirolimus.

48. The method of claim 23, wherein the localized inflammatory or immune related disorder is inflammatory bowel disease, Crohn's disease, ulcerative colitis, or oral lichen planus.
